(19) United States
(12) Patent Application Publication
(10) Pub. No.: US 2026/0234137 A1
(43) Pub. Date: Aug. 13, 2026

(54) BICYCLIC ENHANCERS OF THE PARTICULATE GUANYLYL CYCLASE RECEPTOR A

(71) Applicant: University of Florida Research Foundation, Incorporated, Gainesville, FL (US)

(72) Inventors: Siobhan Malany, Gainesville, FL (US); Satyamaheshwar Peddibhotla, Gainesville, FL (US)

(73) Assignee: University of Florida Research Foundation, Incorporated, Gainesville, FL (US)

(21) Appl. No.: 19/183,299

(22) Filed: Apr. 18, 2025

Related U.S. Application Data
(63) Continuation of application No. PCT/US2023/077286, filed on Oct. 19, 2023.
(60) Provisional application No. 63/417,659, filed on Oct. 19, 2022.

(51) Int. Cl. C07D 417/12; A61K 31/437; A61K 31/454; A61K 31/4545; A61K 31/496; A61K 31/506; A61K 31/5377; C07D 277/82; C07D 417/14; C07D 513/04

(52) U.S. Cl. CPC C07D 417/12; A61K 31/437; A61K 31/454; A61K 31/4545; A61K 31/496; A61K 31/506; A61K 31/5377; C07D 277/82; C07D 417/14; C07D 513/04

(57) ABSTRACT

The present disclosure provides compounds of formulae herein (e.g., Formula (I-a) and (I-b)), and pharmaceutically acceptable salts, solvates, hydrates, polymorphs, co-crystals, tautomers, stereoisomers, isotopically labeled compounds, and prodrugs thereof, which are enhancers of particulate guanylyl cyclase (pGC-A). The present disclosure also provides pharmaceutical compositions and kits comprising the compounds, or pharmaceutically acceptable salts, solvates, hydrates, polymorphs, co-crystals, tautomers, stereoisomers, isotopically labeled compounds, or prodrugs thereof, and methods of treating or preventing diseases by administering to a subject in need thereof the compounds, or pharmaceutically acceptable salts, solvates, hydrates, polymorphs, co-crystals, tautomers, stereoisomers, isotopically labeled compounds, or prodrugs thereof, or pharmaceutical compositions thereof.

### CROSS-REFERENCE TO RELATED APPLICATIONS

This application claims priority under 35 U.S.C. §§ 120 and 365(c) to International Patent Application Serial No. PCT/US2023/077286, filed Oct. 19, 2023, which claims priority under 35 U.S.C. § 119(e) to U.S. Provisional Application Ser. No. 63/417,659, filed Oct. 19, 2022. The entire contents of these applications are incorporated herein by reference in their entireties.

### GOVERNMENT SUPPORT

The invention was made with government support under Grant No. R01 HL158548 awarded by the National Institutes of Health. The government has certain rights in the invention.

### BACKGROUND OF THE INVENTION

Cardiovascular diseases are the leading cause of death in the world. Hypertension, a common cardiac disease, is associated with higher risk for heart failure, myocardial infarction, sudden cardiac death, chronic kidney disease, stroke, and dementia. Reduction of blood pressure can help alleviate the risks associated with hypertension and improve outcomes.

The particulate guanylyl cyclase receptor A (pGC-A, also known as natriuretic peptide receptor 1 (NPR1)) is the molecular target of the cardiac hormones atrial natriuretic peptide (ANP) and B-type natriuretic peptide (BNP). After binding of ANP or BNP, pGC-A produces cyclic 3′,5′-guanosine monophosphate (cGMP) as its second messenger, which regulates blood pressure, elicits cardioprotective responses, and also plays a role in endocrine and renal functions. However, ANP and BNP are rapidly cleared from the body, and so there is a need for strategies to enhance the activity of pGC-A in response to endogenous ANP or BNP.

### SUMMARY OF THE INVENTION

The present disclosure provides small molecule enhancers of the particulate guanylyl cyclase receptor A (pGC-A), which are believed to operate as positive allosteric modulators (PAMs) of pGC-A. Such small molecule enhancers can allow for increased binding of ANP and BNP to pGC-A. Existing peptide-based therapeutics require intravenous or subcutaneous administration, and such small molecule enhancers can instead allow for the development of an oral delivery strategy for chronic therapy. Prior work has been performed to access novel pharmacological enhancement of pGC-A responsiveness to endogenous ANP or BNP. See, e.g., International PCT Application Publication No. WO 2021/243230, published 2 Dec. 2021, which is incorporated herein by reference.

Accordingly, in one aspect, the present disclosure provides compounds of Formula (I-a):

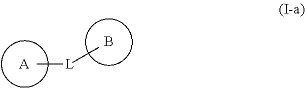

or pharmaceutically acceptable salts, solvates, hydrates, polymorphs, co-crystals, tautomers, stereoisomers, isotopically labeled compounds, or prodrugs thereof, wherein:
   Ring B is of formula:

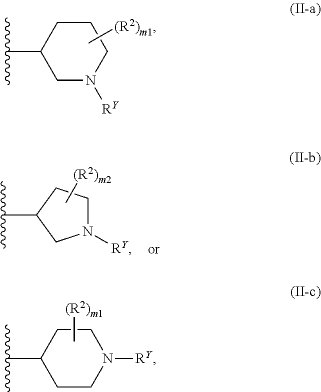

L is of formula:

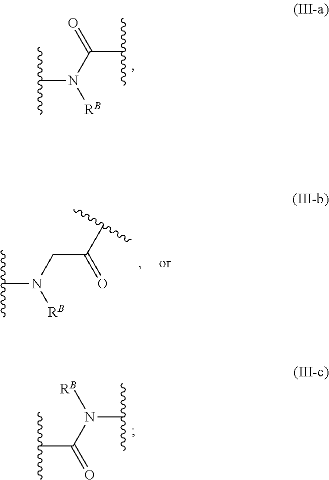

Ring A is of formula:

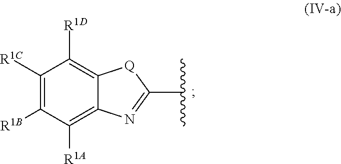

wherein R², R^{B}, R^{Y}, R^{1A}, R^{1B}, R^{1C}, R^{1D}, Q, m1, and m2 are as defined herein.

In another aspect, the present disclosure provides compounds of Formula (I-b):

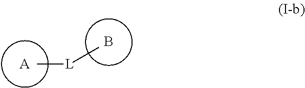

or pharmaceutically acceptable salts, solvates, hydrates, polymorphs, co-crystals, tautomers, stereoisomers, isotopically labeled compounds, or prodrugs thereof, wherein:
   Ring B is optionally substituted 5-6 membered heterocyclyl-R^{X}, wherein the optionally substituted 5-6 membered heterocyclyl comprises at least one ring oxo or thiooxo substituent;
   L is of formula:

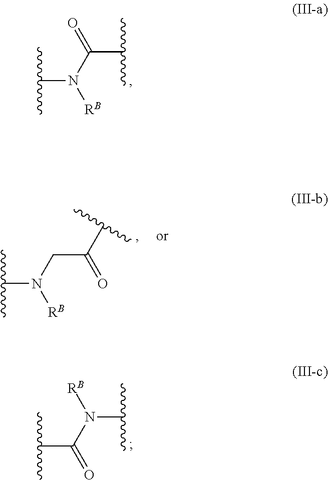

and
Ring A is of formula:

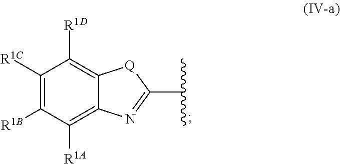

wherein R^{B}, R^{X}, R^{1A}, R^{1B}, R^{1C}, R^{1D}, and Q are as defined herein.

In another aspect, the present disclosure provides pharmaceutical compositions comprising a compound disclosed herein. In some embodiments, the pharmaceutical composition comprises an excipient.

In another aspect, the present disclosure provides methods of treating or preventing a disease in a subject in need thereof, comprising administering to the subject in need thereof a provided compound or pharmaceutical composition. In certain embodiments, the disease is associated with pGC-A (e.g., cardiovascular disease, metabolic disease, kidney disease).

In another aspect, the present disclosure provides methods of modulating particulate guanylyl cyclase-A (pGC-A) in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro.

In another aspect, the present disclosure provides kits comprising a provided compound or pharmaceutical composition disclosed herein and instructions for its use.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

FIG. 1 shows Assay calibration Quality Control. Each lot of ANP was titrated to determine EC₂₀ potentiation concentration to use in combination with test compound and EC₁₀₀ maximal response (top, left) of GC-A receptor. CNP peptide was titrated to determine EC₂₀ and EC₁₀₀ concentrations to use in selectivity assays with GC-B receptor (top, right). MCUF 651 was titrated on each 384-test plate as an internal control compound (bottom, left). Statistics shown in FIG. 2.

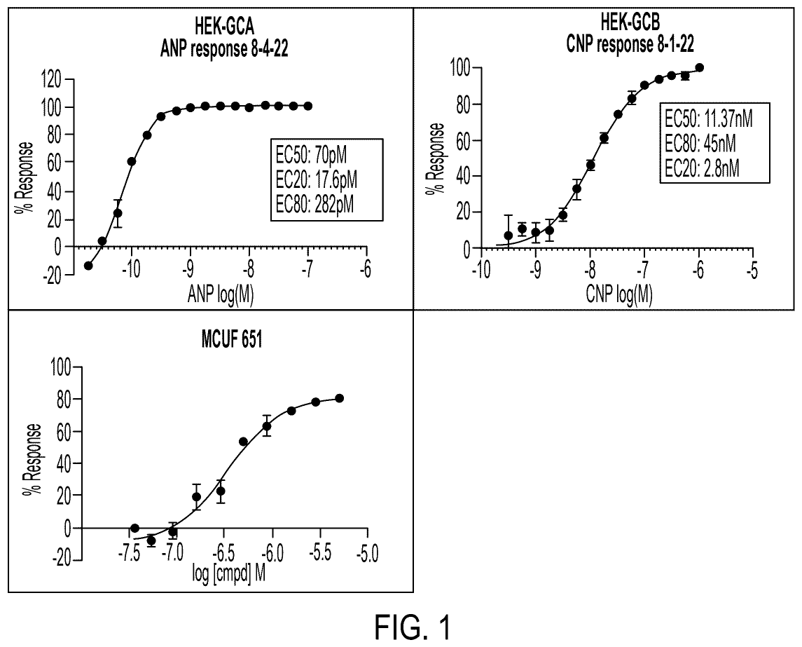

FIG. 2 shows Assay Criteria and Statistics. MCUF 651 was titrated on each 384-test plate as an internal control. Acceptable EC₅₀ values for 651 was between 0.2-0.6 μM (top, left) and Emax values were between 80-100% response (top, right). Signal-to-background determined by the EC₂₀ and EC₁₀₀ values of ANP tracked between 3 and 6-fold for each assay plate (bottom, left).

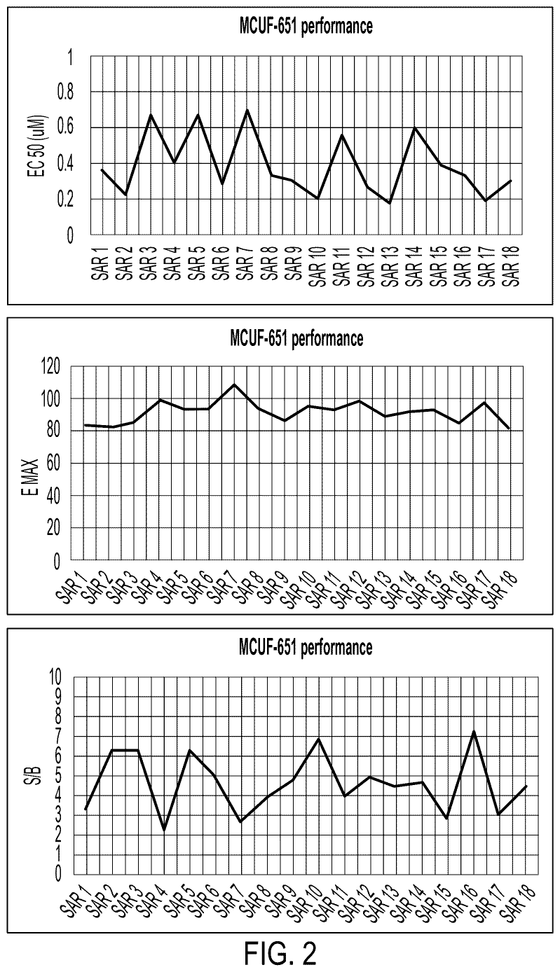

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers. The term “isomers” is intended to include diastereoisomers, enantiomers, regioisomers, structural isomers, rotational isomers, tautomers, and the like. All such isomers of such compounds herein are expressly included in the present invention.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 2 to 20 carbon atoms and one or more carbon-carbon double bonds (e.g., 1, 2, 3, or 4 double bonds). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₂₀ alkenyl. In certain embodiments, the alkenyl group is a substituted C₂₋₂₀ alkenyl. In an alkenyl group, a C═C double bond for which the stereochemistry is not specified (e.g., —CH═CHCH₃ or

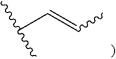

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 11 carbon atoms, at least one double bond, and 1 or more heteroatoms within the parent chain (“heteroC₂₋₁₁ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 carbon atoms, at least one double bond, and 1 heteroatom within the parent chain (“heteroC₂ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₂₀ alkenyl. In certain embodiments, the heteroalkenyl group is a substituted heteroC₂₋₂₀ alkenyl.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₁₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 carbon atoms, at least one triple bond, and 1 heteroatom within the parent chain (“heteroC₂ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₂₀ alkynyl. In certain embodiments, the heteroalkynyl group is a substituted heteroC₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅-10 cycloalkyl”). Examples of C₅₋₆ cycloalkyl groups include cyclopentyl (C₅) and cyclohexyl (C₅). Examples of C₃₋₆ cycloalkyl groups include the aforementioned C₅-6 cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{cc})₂, —P(═O)(OR^{cc})₂, —OP(═O)(R^{cc})₂, —OP(═O)(OR^{cc})₂, —P(═O)(N(R^{bb})₂)₂, —OP(═O)(N(R^{bb})₂)₂, —NR^{bb}P(═O)(R^{cc})₂—NR^{bb}p(═O)(OR^{cc})₂, —NR^{bb}P(═O)(N(R^{bb})₂)₂ —P(R^{cc})₂, —P(OR^{cc})₂, —P(R^{cc})₃X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNRbbS(═O)₂R^{aa}, ═NR^{bb}, or ═NOR^{cc}; wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{cc}, —OC(═O)R^{ee}, —OCO₂R^{ee}, —C(═O)N(R^{ff})₂, —OC(═O)N(R^{ee})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ee})₂, —NR^{ff}C(═NR^{ff})N(R^{ee})₂, —NR^{ff}SO₂R^{cc}, —SO₂N(R^{ee})₂, —SO₂R^{cc}, —SO₂OR^{cc}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{cc}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{a} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{aa})₃⁺X⁻, —OP(═O)(R^{cc})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{a} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphino” refers to the group —P(R^{cc})₂, wherein R^{cc} is as defined herein.

The term “phosphono” refers to the group —(P═O)(OR^{bb})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (“PG,” also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R—, —C(═O)N(R^{bb})₂, or an oxygen protecting group, wherein R^{a} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (“PG,” also referred to herein as an “hydroxyl protecting group”). Oxygen protecting groups include —R^{aa}, —N(R^{b})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{cc})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (“PG,” also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{cc})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p—bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such asp-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound is complexed with the acid in a way that proton transfer (e.g., a complete proton transfer) from the acid to a compound easily occurs at room temperature. In the co-crystal, however, a compound is complexed with the acid in a way that proton transfer from the acid to a herein does not easily occur at room temperature. In certain embodiments, in the co-crystal, there is substantially no proton transfer from the acid to a compound. In certain embodiments, in the co-crystal, there is partial proton transfer from the acid to a compound. Co-crystals may be useful to improve the properties (e.g., solubility, stability, and ease of formulation) of a compound.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers,” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “isotopically labeled compound” refers to a derivative of a compound that only structurally differs from the compound in that at least one atom of the derivative includes at least one isotope enriched above (e.g., enriched 3-, 10-, 30-, 100-, 300-, 1,000-, 3,000- or 10,000-fold above) its natural abundance, whereas each atom of the compound includes isotopes at their natural abundances. In certain embodiments, the isotope enriched above its natural abundance is ²H. In certain embodiments, the isotope enriched above its natural abundance is ¹³C, ¹⁵N, or ¹⁸O.

The term “prodrugs” refers to compounds that have cleavable groups and become by solvolysis or under physiological conditions the compounds described herein, which are pharmaceutically active in vivo. Such examples include choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include, for example, esters prepared by reaction of the parent acid with a suitable alcohol, or amides prepared by reaction of the parent acid compound with a substituted or unsubstituted amine, or acid anhydrides, or mixed anhydrides.

The terms “pharmaceutical composition,” “composition,” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a pharmaceutical composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In some embodiments, the subject is at risk of developing a disease or condition due to environmental factors (e.g., exposure to the sun).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a disease or disorder associated with associated with particulate guanylyl cyclase-A (pGC-A) (e.g., cardiovascular disease, metabolic disease, kidney disease) in a subject in need thereof. In certain embodiments, a therapeutically effective amount is an amount effective for modulating pGC-A in a subject in need thereof or in a cell, tissue, or biological sample. In certain embodiments, a therapeutically effective amount is an amount effective for enhancing an activity of pGC-A (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%). In certain embodiments, a therapeutically effective amount is an amount effective for positive allosteric enhancement of activity of pGC-A. In certain embodiments, a therapeutically effective amount is an amount effective for producing cyclic 3′,5′-guanosine monophosphate (cGMP). In certain embodiments, a therapeutically effective amount is an amount effective for increasing production of cGMP (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%).

A “prophylactically effective amount” of a compound is an amount sufficient to prevent a condition, or one or more signs and/or symptoms associated with the condition or prevent its recurrence. In certain embodiments, the prophylactically effective amount is an amount that improves overall prophylaxis and/or enhances the prophylactic efficacy of another prophylactic agent. In certain embodiments, a prophylactically effective amount is an amount effective for preventing a disease or disorder associated with pGC-A (e.g., cardiovascular disease, metabolic disease, kidney disease) in a subject in need thereof. In certain embodiments, a prophylactically effective amount is an amount effective for reducing the risk of developing a disease or disorder associated with pGC-A (e.g., cardiovascular disease, metabolic disease, kidney disease) in a subject in need thereof. In certain embodiments, a prophylactically effective amount is an amount effective for modulating pGC-A in a subject in need thereof or in a cell, tissue, or biological sample. In certain embodiments, a prophylactically effective amount is an amount effective for enhancing an activity of pGC-A (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%). In certain embodiments, a prophylactically effective amount is an amount effective for positive allosteric enhancement of activity of pGC-A. In certain embodiments, a prophylactically effective amount is an amount effective for producing cyclic 3′,5′-guanosine monophosphate (cGMP). In certain embodiments, a prophylactically effective amount is an amount effective for increasing production of cGMP (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%).

The term “cardiovascular disease” refers to diseases and disorders of the heart and circulatory system. Exemplary cardiovascular diseases, including cholesterol- or lipid-related disorders, include, but are not limited to acute coronary syndrome, angina, arrhythmia, arteriosclerosis, atherosclerosis, atherosclerotic lesions, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, thrombus formation, thromboembolism, primary or recurrent venous thromboembolism (VTE), deep vein thrombosis, pulmonary embolism, non-occlusive venous thrombosis, transitory ischemic attacks, and lipoprotein abnormalities associated with Alzheimer's disease, obesity, diabetes mellitus, syndrome X, impotence, multiple sclerosis, Parkinson's diseases and inflammatory diseases. In some embodiments, the cardiovascular disease is heart failure, cardiomyopathy, hypertension, high blood pressure, or myocardial infarction.

The terms “metabolic disease” and “metabolic disorder” refer to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, hypertriglyceridemia, metabolic syndrome, hyperinsulinemia, acidemia, and obesity. In some embodiments, the metabolic disease is obesity, hypertriglyceridemia, metabolic syndrome, insulin resistance, hyperinsulinemia, diabetes, or acidemia.

The term “kidney disease” refers to a disorder of at least one kidney in a human, wherein the disorder compromises or impairs the function of the kidney(s). In some embodiments, kidney disease is characterized physiologically by the leakage of protein into the urine, or by the excretion of nitrogenous waste. In some embodiments, kidney disease results from a primary pathology of the kidney, such as injury to the glomerulus or tubule, or from damage to another organ, such as the pancreas, which adversely affects the ability of the kidney to perform biological functions, such as the retention of protein. Thus, kidney disease in the human can be the direct or indirect effect of a disease condition which may affect other organs. Exemplary kidney diseases include Abderhalden-Kaufmann-Lignac syndrome (Nephropathic Cystinosis), Abdominal Compartment Syndrome, Acetaminophen-induced Nephrotoxicity, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alagille Syndrome, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Arteriovenous Malformations and Fistulas of the Urologic Tract, Autosomal Dominant Hypocalcemia, Bardet-Biedl Syndrome, Bartter Syndrome, Bath Salts and Acute Kidney Injury, Beer Potomania, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, C1q Nephropathy, C3 Glomerulopathy, C3 Glomerulopathy with Monoclonal Gammopathy, C4 Glomerulopathy, Calcineurin Inhibitor Nephrotoxicity, Callilepsis Laureola Poisoning, Cannabinoid Hyperemesis Acute Renal Failure, Cardiorenal syndrome, Carfilzomib-Indiced Renal Injury, CFHR5 nephropathy, Charcot-Marie-Tooth Disease with Glomerulopathy, Chinese Herbal Medicines and Nephrotoxicity, Cherry Concentrate and Acute Kidney Injury, Cholesterol Emboli, Churg-Strauss syndrome, Chyluria, Ciliopathy, Cocaine and the Kidney, Cold Diuresis, Colistin Nephrotoxicity, Collagenofibrotic Glomerulopathy, Collapsing Glomerulopathy, Collapsing Glomerulopathy Related to CMV, Combination Antiretroviral (cART) Related-Nephropathy, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Copper Sulphate Intoxication, Cortical Necrosis, Crizotinib-related Acute Kidney Injury, Cryocrystalglobulinemia, Cryoglobuinemia, Crystalglobulin-Induced Nephropathy, Crystal-Induced Acute Kidney injury, Crystal-Storing Histiocytosis, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), DHA Crystalline Nephropathy, Dialysis Disequilibrium Syndrome, Diabetes and Diabetic Kidney Disease, Diabetes Insipidus, Dietary Supplements and Renal Failure, Diffuse Mesangial Sclerosis, Diuresis, Djenkol Bean Poisoning (Djenkolism), Down Syndrome and Kidney Disease, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Fluid Overload, Hypervolemia, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Green Smoothie Cleanse Nephropathy, HANAC Syndrome, Harvoni (Ledipasvir with Sofosbuvir)-Induced Renal Injury, Hair Dye Ingestion and Acute Kidney Injury, Hantavirus Infection Podocytopathy, Heat Stress Nephropathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 1β-Associated Kidney Disease, Hepatorenal Syndrome, Herbal Supplements and Kidney Disease, High Altitude Renal Syndrome, High Blood Pressure and Kidney Disease, HIV-Associated Immune Complex Kidney Disease (HIVICK), HIV-Associated Nephropathy (HIVAN), HNF1B-related Autosomal Dominant Tubulointerstitial Kidney Disease, Horseshoe Kidney (Renal Fusion), Hunner's Ulcer, Hydroxychloroquine-induced Renal Phospholipidosis, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia, Hypokalemia-induced renal dysfunction, Hypokalemic Periodic Paralysis, Hypomagnesemia, Hyponatremia, Hypophosphatemia, Hypophosphatemia in Users of Cannabis, Hypertension, Hypertension, Monogenic, Iced Tea Nephropathy, Ifosfamide Nephrotoxicity, IgA Nephropathy, IgG4 Nephropathy, Immersion Diuresis, Immune-Checkpoint Therapy-Related Interstitial Nephritis, Infliximab-Related Renal Disease, Interstitial Cystitis, Painful Bladder Syndrome (Questionnaire), Interstitial Nephritis, Interstitial Nephritis, Karyomegalic, Ivemark's syndrome, JC Virus Nephropathy, Joubert Syndrome, Ketamine-Associated Bladder Dysfunction, Kidney Stones, Nephrolithiasis, Kombucha Tea Toxicity, Lead Nephropathy and Lead-Related Nephrotoxicity, Lecithin Cholesterol Acyltransferase Deficiency (LCAT Deficiency), Leptospirosis Renal Disease, Light Chain Deposition Disease, Monoclonal Immunoglobulin Deposition Disease, Light Chain Proximal Tubulopathy, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lupus Podocytopathy, Lyme Disease-Associated Glomerulonephritis, Lysinuric Protein Intolerance, Lysozyme Nephropathy, Malarial Nephropathy, Malignancy-Associated Renal Disease, Malignant Hypertension, Malakoplakia, McKittrick-Wheelock Syndrome, MDMA (Molly; Ecstacy; 3,4-Methylenedioxymethamphetamine) and Kidney Failure, Meatal Stenosis, Medullary Cystic Kidney Disease, Urolodulin-Associated Nephropathy, Juvenile Hyperuricemic Nephropathy Type 1, Medullary Sponge Kidney, Megaureter, Melamine Toxicity and the Kidney, MELAS Syndrome, Membranoproliferative Glomerulonephritis, Membranous Nephropathy, Membranous-like Glomerulopathy with Masked IgG Kappa Deposits, MesoAmerican Nephropathy, Metabolic Acidosis, Metabolic Alkalosis, Methotrexate-related Renal Failure, Microscopic Polyangiitis, Milk-alkalai syndrome, Minimal Change Disease, Monoclonal Gammopathy of Renal Significance, Dysproteinemia, Mouthwash Toxicity, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrogenic Systemic Fibrosis, Nephroptosis (Floating Kidney, Renal Ptosis), Nephrotic Syndrome, Neurogenic Bladder, 9/11 and Kidney Disease, Nodular Glomerulosclerosis, Non-Gonococcal Urethritis, Nutcracker syndrome, Oligomeganephronia, Orofaciodigital Syndrome, Orotic Aciduria, Orthostatic Hypotension, Orthostatic Proteinuria, Osmotic Diuresis, Osmotic Nephrosis, Ovarian Hyperstimulation Syndrome, Oxalate Nephropathy, Page Kidney, Papillary Necrosis, Papillorenal Syndrome (Renal-Coloboma Syndrome, Isolated Renal Hypoplasia), PARN Mutations and Kidney Disease, Parvovirus B19 and the Kidney, The Peritoneal-Renal Syndrome, POEMS Syndrome, Posterior Urethral Valve, Podocyte Infolding Glomerulopathy, Post-infectious Glomerulonephritis, Post-streptococcal Glomerulonephritis, Post-infectious Glomerulonephritis, Atypical, Post-Infectious Glomerulonephritis (IgA-Dominant), Mimicking IgA Nephropathy, Polyarteritis Nodosa, Polycystic Kidney Disease, Posterior Urethral Valves, Post-Obstructive Diuresis, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Pyridium and Kidney Failure, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Dissection, Spontaneous, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Mutations and Autosomal Dominant Tubulointerstitial Kidney Disease, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Surgery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjogren's Syndrome and Renal Disease, Synthetic Cannabinoid Use and Acute Kidney Injury, Kidney Disease Following Hematopoietic Cell Transplantation, Kidney Disease Related to Stem Cell Transplantation, TAFRO Syndrome, Tea and Toast Hyponatremia, Tenofovir-Induced Nephrotoxicity, Thin Basement Membrane Disease, Benign Familial Hematuria, Thrombotic Microangiopathy Associated with Monoclonal Gammopathy, Trench Nephritis, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Urogenital Fistula, Uromodulin-Associated Kidney Disease, Vancomycin-Associated Cast Nephropathy, Vasomotor Nephropathy, Vesicointestinal Fistula, Vesicoureteral Reflux, VEGF Inhibition and Renal Thrombotic Microangiopathy, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, Wunderlich syndrome, Zellweger Syndrome, or Cerebrohepatorenal Syndrome. In some embodiments, the kidney disease is nephropathy, acute renal failure, chronic kidney disease, or diabetic kidney disease.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, the present disclosure provides a compound of Formula (I-a):

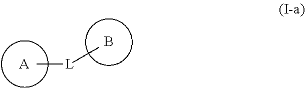

or a pharmaceutically acceptable salt, solvate, hydrate, polymorph, co-crystal, tautomer, stereoisomer, isotopically labeled compound, or prodrug thereof, wherein:
   Ring B is of formula:

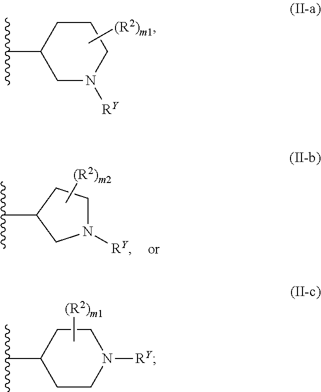

L is of formula:

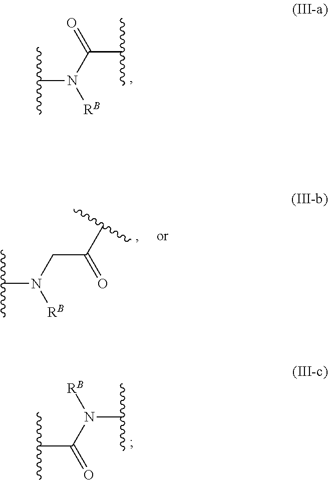

Ring A is of formula:

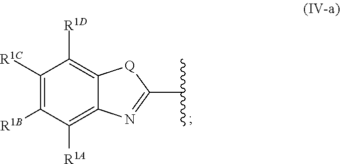

Q is oxygen, sulfur, or NR^{B};
R^{1A} is halogen;
each of R^{1B}, R^{1C}, and R^{1D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; or R^{1B} and R^{1C} or R^{1C} and R^{1D} are taken together to form an optionally substituted carbocyclyl, R^{1B} and R^{1C} or R^{1C} and R^{1D} are taken together to form an optionally substituted heterocyclyl, R^{1B} and R^{1C} or R^{1C} and R^{1D} are taken together to form an optionally substituted aryl, or R^{1B} and R^{1C} or R^{1C} and R^{1D} are taken together to form an optionally substituted heteroaryl;
each occurrence of R² is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A} —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; or two occurrences of R² are taken together to form ═O or ═S, two occurrences of R² are taken together to form an optionally substituted carbocyclyl, or two occurrences of R² are taken together to form an optionally substituted heterocyclyl;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring;
each occurrence of R^{B} is hydrogen, optionally substituted alkyl, optionally substituted acyl, or a nitrogen protecting group;
R^{Y} is optionally substituted carbocyclyl, optionally substituted heterocyclyl containing a single nitrogen atom in the heterocyclyl, optionally substituted aryl, optionally substituted heteroaryl, optionally substituted alkyl substituted with optionally substituted heteroaryl, or optionally substituted alkyl substituted with optionally substituted heterocyclyl comprising at least one ring oxo or thiooxo substituent in the heterocyclyl;
m1 is 0, 1, 2, 3, 4, 5, 6, 7, 8, or 9; and
m2 is 0, 1, 2, 3, 4, 5, 6, or 7.

In another aspect, the present disclosure provides a compound of Formula (I-b):

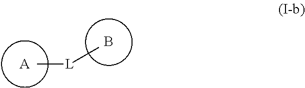

or a pharmaceutically acceptable salt, solvate, hydrate, polymorph, co-crystal, tautomer, stereoisomer, isotopically labeled compound, or prodrug thereof, wherein:
   Ring B is optionally substituted 5-6 membered heterocyclyl-R^{X1}, wherein the optionally substituted 5-6 membered heterocyclyl comprises at least one ring oxo or thiooxo substituent;
   L is of formula:

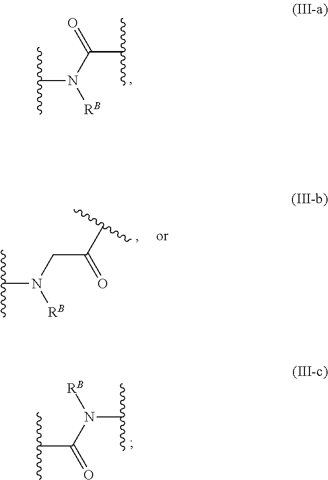

Ring A is of formula:

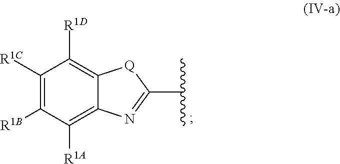

Q is oxygen, sulfur, or NR^{B};
R^{1A} is halogen;
each of R^{B}, R^{1C}, and R^{1D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A} —NR^{A}C(═O)OR^{A} —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; or R^{1B} and R^{1C} or R^{1C} and R^{1D} are taken together to form an optionally substituted carbocyclyl, R^{1B} and R^{1C} or R^{1C} and R^{1D} are taken together to form an optionally substituted heterocyclyl, R^{1B} and R^{1C} or R^{1C} and R^{1D} are taken together to form an optionally substituted aryl, or R^{1B} and R^{1C} or R^{1C} and R^{1D} are taken together to form an optionally substituted heteroaryl;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring;
each occurrence of R^{B} is hydrogen, optionally substituted alkyl, optionally substituted acyl, or a nitrogen protecting group; and
R^{X} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

### L

In certain embodiments, L is of formula

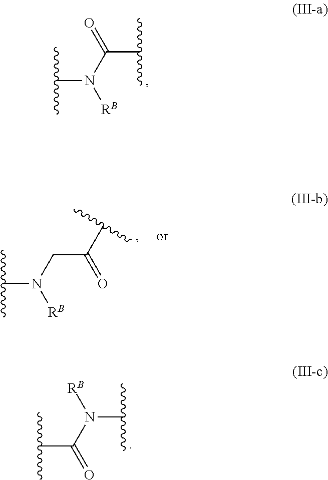

In certain embodiments, L is of formula

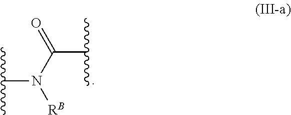

In certain embodiments, L is of formula

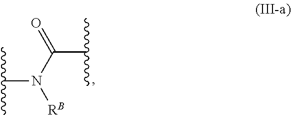

and R^{B} is hydrogen, substituted or unsubstituted alkyl, substituted or unsubstituted acyl, or a nitrogen protecting group. In certain embodiments, L is of formula

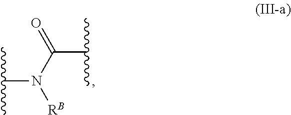

and R^{B} is hydrogen or optionally substituted alkyl. In certain embodiments, L is of formula

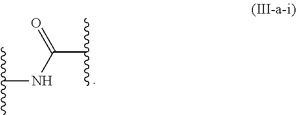 [SMILES: *NC(*)=O]

In certain embodiments, L is of formula

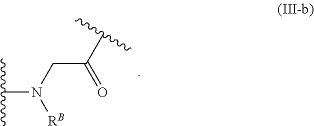

In certain embodiments, L is of formula

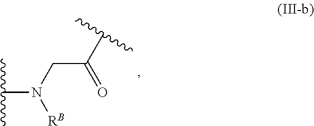

and R^{B} is hydrogen, substituted or unsubstituted alkyl, substituted or unsubstituted acyl, or a nitrogen protecting group. In certain embodiments, L is of formula

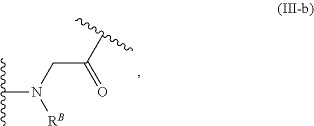

and R^{B} is hydrogen or optionally substituted alkyl. In certain embodiments, L is of formula

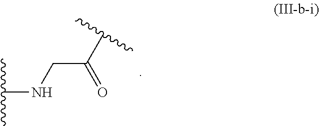 [SMILES: *NCC(*)=O]

In certain embodiments, L is of formula

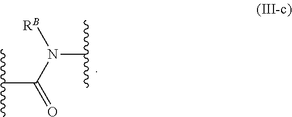

In certain embodiments, L is of formula

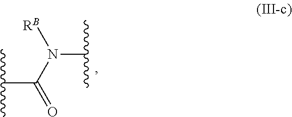

and R^{B} is hydrogen, substituted or unsubstituted alkyl, substituted or unsubstituted acyl, or a nitrogen protecting group. In certain embodiments, L is of formula

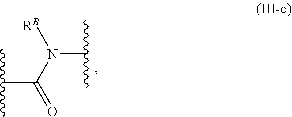

and R^{B} is hydrogen or optionally substituted alkyl. In certain embodiments, L is of formula

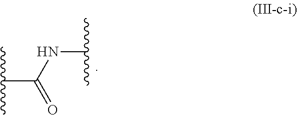 [SMILES: *NC(*)=O]

Ring A, Q, R^{1A}, R^{1B}, R^{1C}, and R^{1D}

In certain embodiments, Ring A is of formula

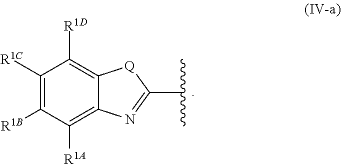

In certain embodiments, Ring A is of formula

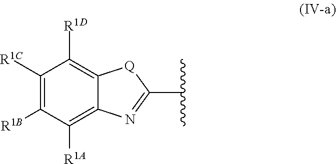

and Q is oxygen. In certain embodiments, Ring A is of formula

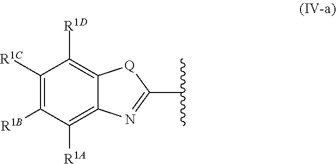

and Q is sulfur. In certain embodiments, Ring A is of formula

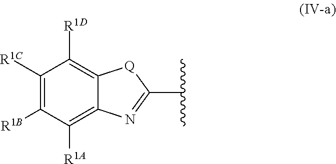

and Q is NR^{B}. In certain embodiments, Ring A is of formula

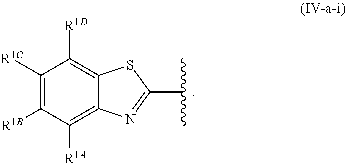

In certain embodiments, Ring A is of formula

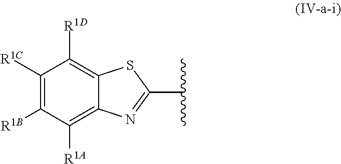

and R^{1A} is halogen. In certain embodiments, Ring A is of formula

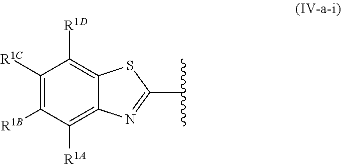

and R^{1C} is halogen. In certain embodiments, Ring A is of formula

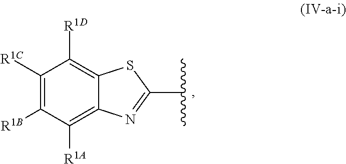

R^{1A} is halogen, and R^{1C} is halogen. In certain embodiments, Ring A is of formula

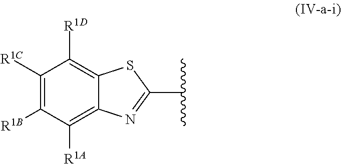

and R^{1C} is fluoro. In certain embodiments, Ring A is of formula

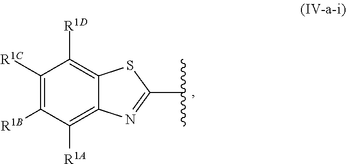

R^{1A} is fluoro, and R^{1C} is fluoro. In certain embodiments, Ring A is of formula

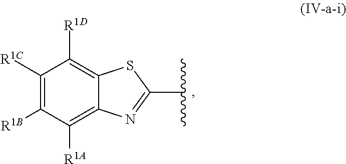

R^{1A} is chloro, and R^{1C} is fluoro. In certain embodiments, Ring A is of formula

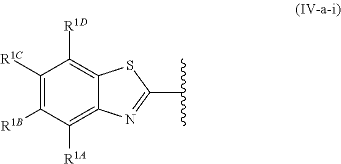

and R^{1C} is chloro. In certain embodiments, Ring A is of formula

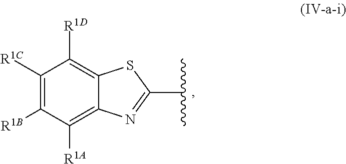

R^{1A} is fluoro, and R^{1C} is chloro. In certain embodiments, Ring A is of formula

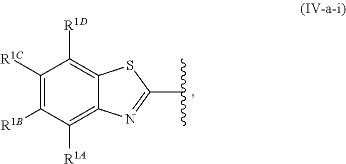

R^{1A} is chloro, and R^{1C} is chloro. In certain embodiments, Ring A is of formula

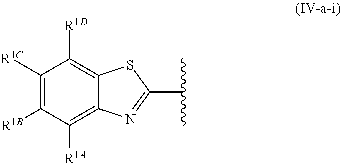

and R^{1C} is optionally substituted alkyl or —OR^{A}. In certain embodiments, Ring A is of formula

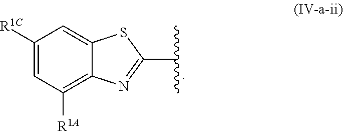

In certain embodiments, Ring A is of formula

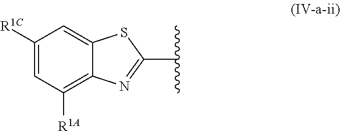

and R^{1A} is halogen. In certain embodiments, Ring A is of formula

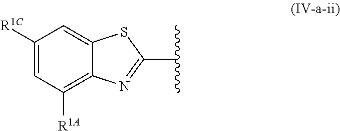

and R^{1C} is halogen. In certain embodiments, Ring A is of formula

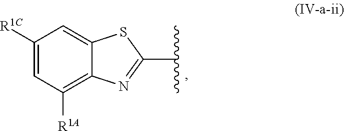

R^{1A} is halogen, and R^{1C} is halogen. In certain embodiments, Ring A is of formula

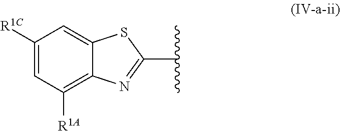

and R^{1C} is fluoro. In certain embodiments, Ring A is of formula

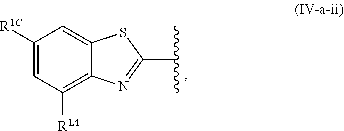

R^{1A} is fluoro, and R^{1C} is fluoro. In certain embodiments, Ring A is of formula

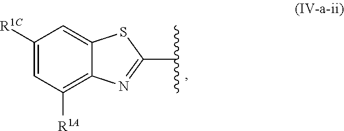

R^{1A} is chloro, and R^{1C} is fluoro. In certain embodiments, Ring A is of formula

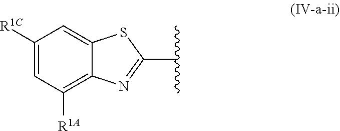

and R^{1C} is chloro. In certain embodiments, Ring A is of formula

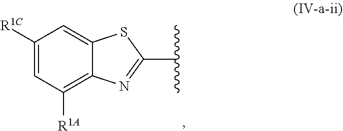

R^{1A} is fluoro, and R^{1C} is chloro. In certain embodiments, Ring A is of formula

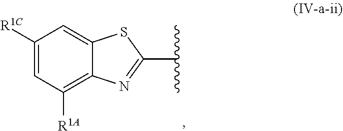

R^{1A} is chloro, and R^{1C} is chloro. In certain embodiments, Ring A is of formula

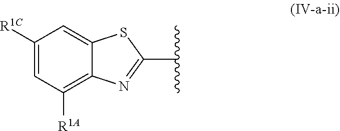

and R^{1C} is optionally substituted alkyl or —OR^{A}.

In certain embodiments, Q is oxygen, sulfur, or NR^{B}. In certain embodiments, Q is oxygen. In certain embodiments, Q is sulfur. In certain embodiments, Q is NR^{B}.

In certain embodiments, R^{1A} is halogen. In certain embodiments, R^{1A} is fluoro. In certain embodiments, R^{1A} is chloro. In certain embodiments, R^{1A} is bromo. In certain embodiments, R^{1A} is iodo. In certain embodiments, R^{1A} is halogen and R^{1C} is halogen. In certain embodiments, R^{1A} is fluoro. In certain embodiments, R^{1A} is fluoro and R^{1C} is fluoro. In certain embodiments, R^{1A} is fluoro and R^{1C} is chloro. In certain embodiments, R^{1A} is fluoro and R^{1C} is bromo. In certain embodiments, R^{1A} is fluoro and R^{1C} is iodo. In certain embodiments, R^{1A} is chloro. In certain embodiments, R^{1A} is chloro and R^{1C} is fluoro. In certain embodiments, R^{1A} is chloro and R^{1C} is chloro. In certain embodiments, R^{1A} is chloro and R^{1C} is bromo. In certain embodiments, R^{1A} is chloro and R^{1C} is iodo. In certain embodiments, R^{1A} is bromo. In certain embodiments, R^{1A} is bromo and R^{1C} is fluoro. In certain embodiments, R^{1A} is bromo and R^{1C} is chloro. In certain embodiments, R^{1A} is bromo and R^{1C} is bromo. In certain embodiments, R^{1A} is bromo and R^{1C} is iodo. In certain embodiments, R^{1A} is iodo. In certain embodiments, R^{1A} is iodo and R^{1C} is fluoro. In certain embodiments, R^{1A} is iodo and R^{1C} is chloro. In certain embodiments, R^{1A} is iodo and R^{1C} is bromo. In certain embodiments, R^{1A} is iodo and R^{1C} is iodo. In certain embodiments, R^{1A} is halogen and R^{1C} is optionally substituted alkyl or —OR^{A}. In certain embodiments, R^{1A} is fluoro and R^{1C} is optionally substituted alkyl or —OR^{A}. In certain embodiments, R^{1A} is chloro and R^{1C} is optionally substituted alkyl or —OR^{A}. In certain embodiments, R^{1A} is bromo and R^{1C} is optionally substituted alkyl or —OR^{A}. In certain embodiments, R^{1A} is iodo and R^{1C} is optionally substituted alkyl or —OR^{A}.

In certain embodiments, each of R^{1B}, R^{1C}, R^{1D}, and R^{1E} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; or R^{1B} and R^{1C} or R^{1C} and R^{1D} are taken together to form an optionally substituted carbocyclyl, R^{1B} and R^{1C} or R^{1C} and R^{1D} are taken together to form an optionally substituted heterocyclyl, R^{1B} and R^{1C} or R^{1C} and R^{1D} are taken together to form an optionally substituted aryl, or R^{1B} and R^{1C} or Ric and R^{1D} are taken together to form an optionally substituted heteroaryl.

In certain embodiments, R^{1B} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, R^{1B} is hydrogen. In certain embodiments, R^{1B} is halogen. In certain embodiments, R^{1B} is fluoro. In certain embodiments, R^{1B} is chloro. In certain embodiments, R^{1B} is bromo. In certain embodiments, R^{1B} is iodo. In certain embodiments, R^{1B} is optionally substituted alkyl. In certain embodiments, R^{1B} is optionally substituted heteroalkyl. In certain embodiments, R^{1B} is —OR^{A}.

In certain embodiments, R^{1C} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, R^{1C} is hydrogen. In certain embodiments, R^{1C} is halogen. In certain embodiments, R^{1C} is fluoro. In certain embodiments, R^{1C} is chloro. In certain embodiments, R^{1C} is bromo. In certain embodiments, R^{1C} is iodo. In certain embodiments, R^{1C} is optionally substituted alkyl. In certain embodiments, R^{1C} is optionally substituted heteroalkyl. In certain embodiments, Ric is —OR^{A}.

In certain embodiments, R^{1D} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, RD is hydrogen. In certain embodiments, R^{1D} is halogen. In certain embodiments, R^{1D} is fluoro.

In certain embodiments, R^{1D} is chloro. In certain embodiments, R^{1D} is bromo. In certain embodiments, R^{1D} is iodo. In certain embodiments, R^{1D} is optionally substituted alkyl. In certain embodiments, R^{1D} is optionally substituted heteroalkyl. In certain embodiments, R^{1D} is —OR^{A}.

R^{A} and R^{B}

In certain embodiments, each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is hydrogen. In certain embodiments, at least one occurrence of R^{A} is optionally substituted acyl.

In certain embodiments, at least one occurrence of R^{A} is optionally substituted C₁₋₁₂ alkyl. In certain embodiments, at least one occurrence of R^{A} is optionally substituted C₁₋₆ alkyl. In certain embodiments, at least one occurrence of R^{A} is unsubstituted C₁₋₆ alkyl. In certain embodiments, at least one occurrence of R^{A} is substituted C₁₋₆ alkyl. In certain embodiments, at least one occurrence of R^{A} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least one occurrence of R^{A} is optionally substituted C₂₋₁₂ alkenyl. In certain embodiments, at least one occurrence of R^{A} is optionally substituted C₂₋₆ alkenyl. In certain embodiments, at least one occurrence of R^{A} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R^{A} is optionally substituted C₂₋₁₂ alkynyl. In certain embodiments, at least one occurrence of R^{A} is optionally substituted C₂₋₆ alkynyl. In certain embodiments, at least one occurrence of R^{A} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R^{A} is optionally substituted heteroC₁₋₁₂ alkyl. In certain embodiments, at least one occurrence of R^{A} is optionally substituted heteroC₁₋₆ alkyl. In certain embodiments, at least one occurrence of R^{A} is optionally substituted heteroC₁₋₁₂ alkenyl. In certain embodiments, at least one occurrence of R^{A} is optionally substituted heteroC₁₋₆ alkenyl. In certain embodiments, at least one occurrence of R^{A} is optionally substituted heteroC₁₋₁₂ alkynyl. In certain embodiments, at least one occurrence of R^{A} is optionally substituted heteroC₁₋₆ alkynyl. In certain embodiments, at least one occurrence of R^{A} is optionally substituted C₃₋₁₄ cycloalkyl. In certain embodiments, at least one occurrence of R^{A} is optionally substituted 5-10 membered heterocyclyl. In certain embodiments, at least one occurrence of R^{A} is optionally substituted 6-14 membered aryl. In certain embodiments, at least one occurrence of R^{A} is optionally substituted 5-14 membered heteroaryl. In certain embodiments, at least one occurrence of R^{A} is a nitrogen protecting group when attached to a nitrogen atom. In certain embodiments, at least one occurrence of R^{A} is an oxygen protecting group when attached to an oxygen atom. In certain embodiments, at least one occurrence of R^{A} is a sulfur protecting group when attached to a sulfur atom. In certain embodiments, at least two occurrences of R^{A} are joined together with their intervening atom to form an optionally substituted 5-10 membered heterocyclic ring. In certain embodiments, at least two occurrences of R^{A} are joined together with their intervening atom to form an optionally substituted 5-14 membered heteroaryl ring.

In certain embodiments, each occurrence of R^{B} is hydrogen, optionally substituted alkyl, optionally substituted acyl, or a nitrogen protecting group. In certain embodiments, at least one occurrence of R^{B} is hydrogen, optionally substituted alkyl, optionally substituted acyl, or a nitrogen protecting group. In certain embodiments, at least one occurrence of R^{B} is hydrogen. In certain embodiments, at least one occurrence of R^{B} is optionally substituted alkyl. In certain embodiments, at least one occurrence of R^{B} is optionally substituted C₁₋₁₀ alkyl. In certain embodiments, at least one occurrence of R^{B} is optionally substituted C₁₋₆ alkyl. In certain embodiments, at least one occurrence of R^{B} is optionally substituted C₁₋₃ alkyl. In certain embodiments, at least one occurrence of R^{B} is optionally substituted C₁₋₃ alkyl. In certain embodiments, R^{B} is optionally substituted methyl, optionally substituted ethyl, optionally substituted n-propyl, or optionally substituted isopropyl. In certain embodiments, at least one occurrence of R^{B} is optionally substituted acyl. In certain embodiments, at least one occurrence of R^{B} is a nitrogen protecting group.

### Formula (I-a)

Ring B, R^{Y}, R², m1, and m2

In certain embodiments, Ring B is of formula

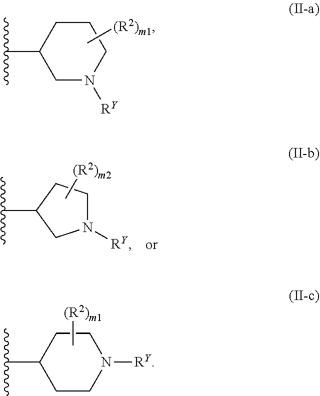

In certain embodiments, Ring B is of formula

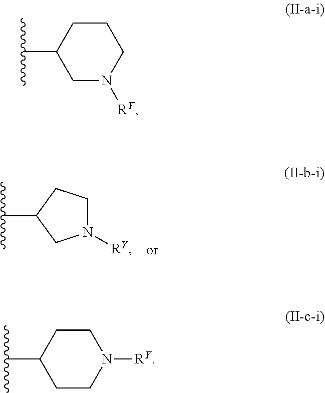

In certain embodiments Ring B is of formula

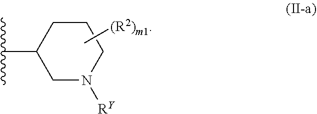

In certain embodiments, Ring B is of formula

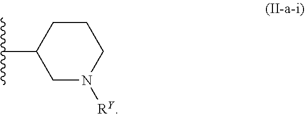

In certain embodiments, Ring B is of formula

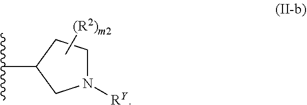

In certain embodiments, Ring B is of formula

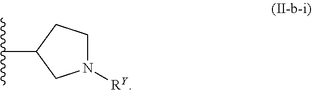

In certain embodiments, Ring B is of formula

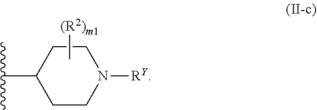

In certain embodiments, Ring B is of formula

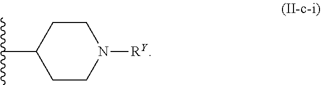

In certain embodiments, Ring A is of formula

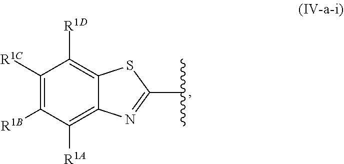

and Ring B is of formula

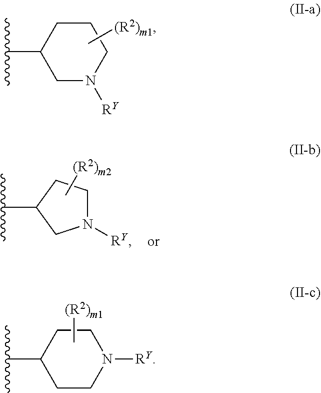

In certain embodiments, Ring A is of formula

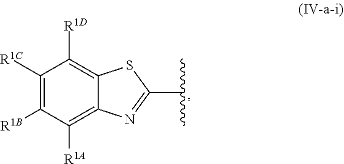

and Ring B is of formula

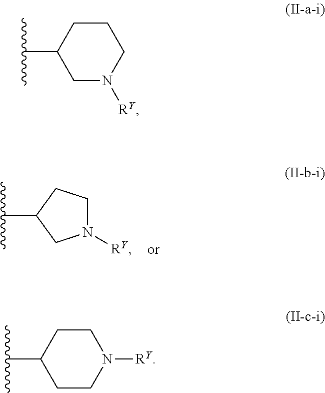

In certain embodiments, Ring A is of formula

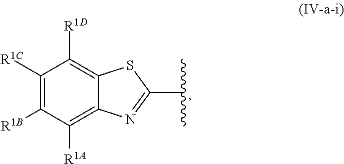

L is of formula

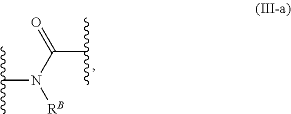

and Ring B is of formula

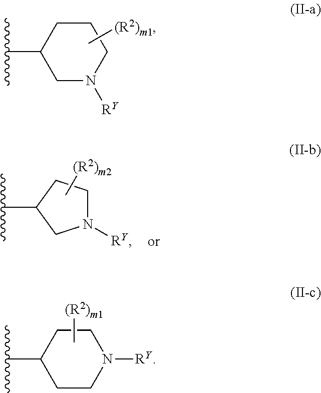

In certain embodiments, Ring A is of formula

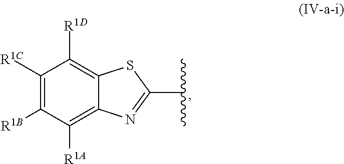

L is of formula

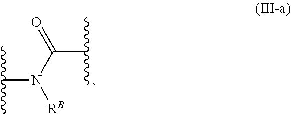

and Ring B is of formula

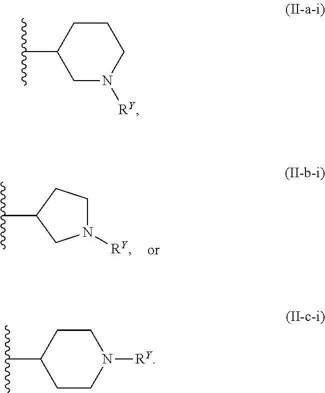

In certain embodiments, R^{Y} is optionally substituted carbocyclyl, optionally substituted heterocyclyl containing a single nitrogen atom in the heterocyclyl, optionally substituted aryl, optionally substituted heteroaryl, optionally substituted alkyl substituted with optionally substituted heteroaryl, or optionally substituted alkyl substituted with optionally substituted heterocyclyl comprising at least one ring oxo or thiooxo substituent in the heterocyclyl.

In certain embodiments, R^{Y} is optionally substituted carbocyclyl. In certain embodiments, R^{Y} is substituted or unsubstituted, monocyclic carbocyclyl (e.g., substituted or unsubstituted, monocyclic, 3- to 10-membered carbocyclyl). In certain embodiments, R^{Y} is substituted or unsubstituted, polycyclic carbocyclyl (e.g., substituted or unsubstituted, bicyclic or tricyclic, 3- to 14-membered carbocyclyl). In certain embodiments, R^{Y} is optionally substituted saturated carbocyclyl. In certain embodiments, R, is substituted or unsubstituted cyclopropyl, substituted or unsubstituted cyclobutyl, substituted or unsubstituted cyclopentyl, substituted or unsubstituted cyclohexyl, substituted or unsubstituted cycloheptyl, substituted or unsubstituted cyclooctyl, substituted or unsubstituted cyclononyl, or substituted or unsubstituted cyclodecyl. In certain embodiments, R^{Y} is substituted carbocyclyl. In certain embodiments, R^{Y} is substituted saturated carbocyclyl. In certain embodiments, R^{Y} is substituted cyclopropyl, substituted cyclobutyl, substituted cyclopentyl, substituted cyclohexyl, substituted cycloheptyl, substituted cyclooctyl, substituted cyclononyl, or substituted cyclodecyl. In certain embodiments, R^{Y} is substituted cyclohexyl. In certain embodiments, R^{Y} is carbocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, R^{Y} is carbocyclyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, R is saturated carbocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, R^{Y} is saturated carbocyclyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, R^{Y} is cyclohexyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, R^{Y} is unsubstituted carbocyclyl. In certain embodiments, R^{Y} is unsubstituted saturated carbocyclyl. In certain embodiments, R^{Y} is unsubstituted cyclopropyl, unsubstituted cyclobutyl, unsubstituted cyclopentyl, unsubstituted cyclohexyl, unsubstituted cycloheptyl, unsubstituted cyclooctyl, unsubstituted cyclononyl, or unsubstituted cyclodecyl. In certain embodiments, R^{Y} is unsubstituted cyclohexyl. In certain embodiments, R^{Y} is optionally substituted carbocyclyl comprising only one C═C bond and no C═C bonds in the carbocyclic ring system. In certain embodiments, R^{Y} is optionally substituted monocyclic, 3- to 10-membered carbocyclyl comprising only one C═C bond and no C═C bonds in the carbocyclic ring system.

In certain embodiments, R^{Y} is optionally substituted carbocyclyl, optionally substituted heterocyclyl containing a single nitrogen atom in the heterocyclyl, optionally substituted aryl, optionally substituted heteroaryl, optionally substituted alkyl substituted with optionally substituted heteroaryl, or optionally substituted alkyl substituted with optionally substituted heterocyclyl comprising at least one ring oxo or thiooxo substituent in the heterocyclyl; and R^{Y} is not hydrogen, optionally substituted C₁₋₆ alkyl, optionally substituted C₁₋₆ haloalkyl, optionally substituted C₂₋₆ alkenyl, optionally substituted C₂₋₆ alkynyl, acyl, or sulfonyl. In certain embodiments, R^{Y} is not hydrogen. In certain embodiments, R^{Y} is not optionally substituted C₁₋₆ alkyl. In certain embodiments, R^{Y} is not optionally substituted C₁₋₆ haloalkyl. In certain embodiments, R^{Y} is not optionally substituted C₂₋₆ alkenyl. In certain embodiments, R^{Y} is not optionally substituted C₂₋₆ alkynyl. In certain embodiments, R^{Y} is not acyl. In certain embodiments, R^{Y} is not sulfonyl.

In certain embodiments, Ring B is of formula

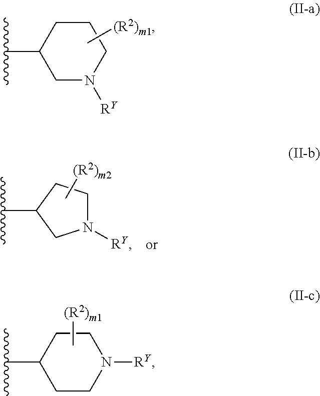

and R^{Y} is optionally substituted carbocyclyl. In certain embodiments, Ring B is of formula

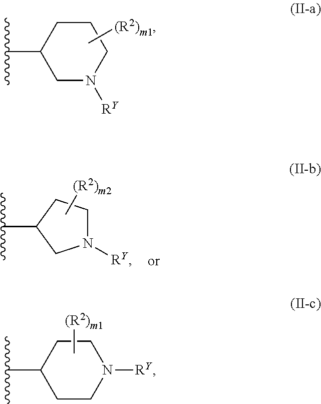

and R^{Y} is substituted or unsubstituted cyclopropyl, substituted or unsubstituted cyclobutyl, substituted or unsubstituted cyclopentyl, substituted or unsubstituted cyclohexyl, substituted or unsubstituted cycloheptyl, substituted or unsubstituted cyclooctyl, substituted or unsubstituted cyclononyl, or substituted or unsubstituted cyclodecyl. In certain embodiments, Ring B is of formula

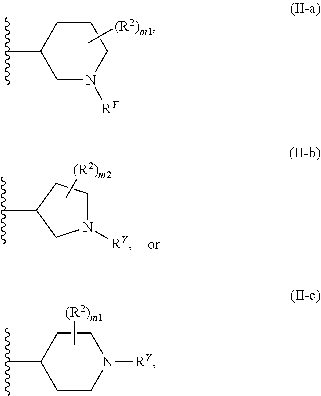

and R^{Y} is carbocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

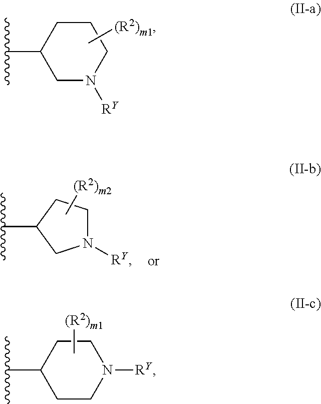

and R^{Y} is carbocyclyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

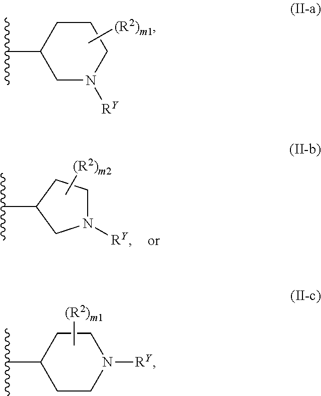

and R^{Y} is saturated carbocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A} —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

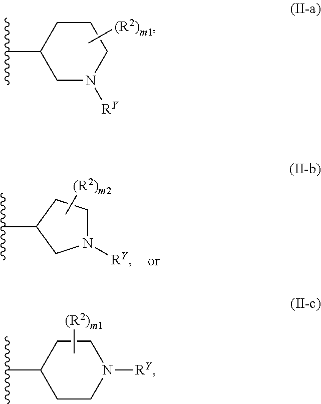

and R^{Y} is saturated carbocyclyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

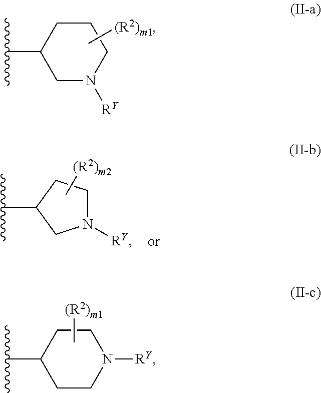

and R^{Y} is cyclohexyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

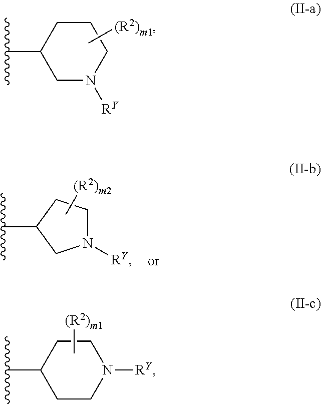

and R^{Y} is unsubstituted carbocyclyl. In certain embodiments, Ring B is of formula

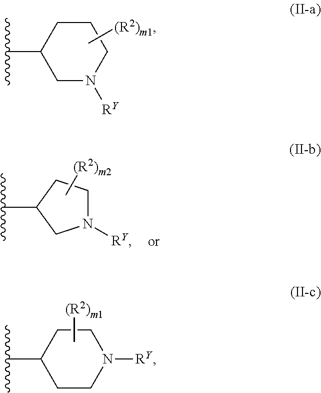

and R^{Y} is unsubstituted cyclopropyl, unsubstituted cyclobutyl, unsubstituted cyclopentyl, unsubstituted cyclohexyl, unsubstituted cycloheptyl, unsubstituted cyclooctyl, unsubstituted cyclononyl, or unsubstituted cyclodecyl. In certain embodiments, Ring B is of formula

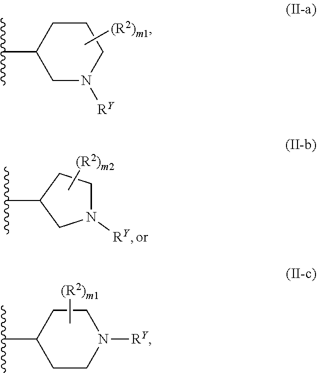

and R^{Y} is unsubstituted cyclohexyl. In certain embodiments, Ring B is of formula

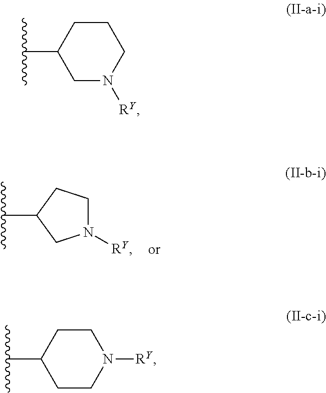

and R^{Y} is optionally substituted carbocyclyl. In certain embodiments, Ring B is of formula

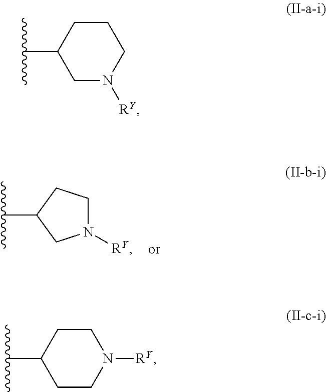

and R^{Y} is substituted or unsubstituted cyclopropyl, substituted or unsubstituted cyclobutyl, substituted or unsubstituted cyclopentyl, substituted or unsubstituted cyclohexyl, substituted or unsubstituted cycloheptyl, substituted or unsubstituted cyclooctyl, substituted or unsubstituted cyclononyl, or substituted or unsubstituted cyclodecyl. In certain embodiments, Ring B is of formula

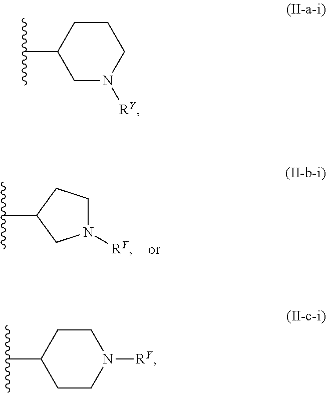

and R^{Y} is carbocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

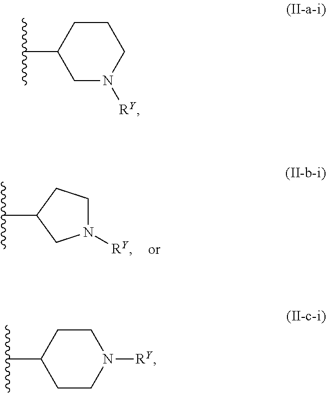

and R^{Y} is carbocyclyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

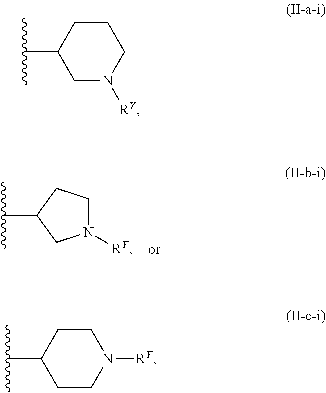

and R^{Y} is saturated carbocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

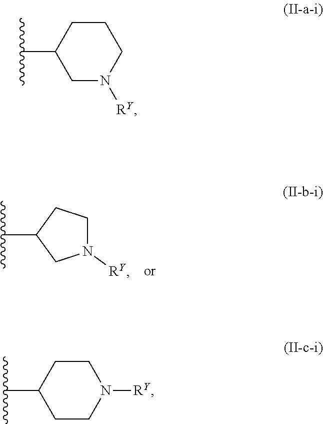

and R^{Y} is saturated carbocyclyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

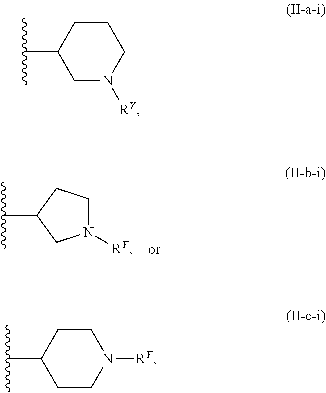

and R^{Y} is cyclohexyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

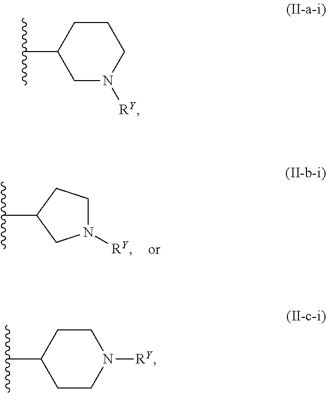

and R^{Y} is unsubstituted carbocyclyl. In certain embodiments, Ring B is of formula

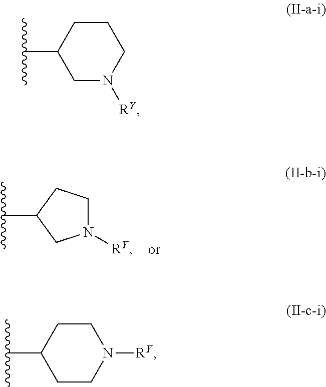

and R^{Y} is unsubstituted cyclopropyl, unsubstituted cyclobutyl, unsubstituted cyclopentyl, unsubstituted cyclohexyl, unsubstituted cycloheptyl, unsubstituted cyclooctyl, unsubstituted cyclononyl, or unsubstituted cyclodecyl. In certain embodiments, Ring B is of formula

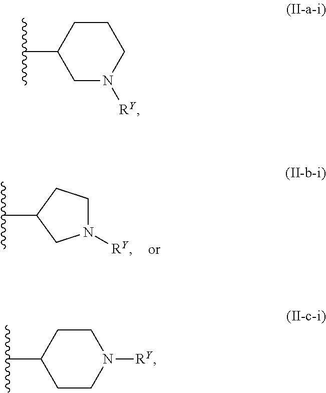

and R^{Y} is unsubstituted cyclohexyl.

In certain embodiments, R^{Y} is optionally substituted heterocyclyl containing a single nitrogen atom in the heterocyclyl. In certain embodiments, R^{Y} is optionally substituted 3-14 membered heterocyclyl containing a single nitrogen atom in the heterocyclyl. In certain embodiments, R^{Y} is substituted or unsubstituted, monocyclic heterocyclyl (e.g., substituted or unsubstituted, 3- to 7-membered, monocyclic heterocyclyl) containing a single nitrogen atom in the heterocyclyl. In certain embodiments, R^{Y} is optionally substituted aziridinyl, optionally substituted azetidinyl, optionally substituted pyrrolidinyl, optionally substituted piperidinyl, or optionally substituted azepanyl. In certain embodiments, R^{Y} is optionally substituted azetidinyl, optionally substituted pyrrolidinyl, or optionally substituted piperidinyl. In certain embodiments, R^{Y} is substituted aziridinyl, substituted azetidinyl, substituted pyrrolidinyl, substituted piperidinyl, or substituted azepanyl. In certain embodiments, R^{Y} is substituted azetidinyl, substituted pyrrolidinyl, or substituted piperidinyl. In certain embodiments, R^{Y} is heterocyclyl containing a single nitrogen atom in the heterocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, R^{Y} is heterocyclyl containing a single nitrogen atom in the heterocyclyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, R^{Y} is aziridinyl, azetidinyl, pyrrolidinyl, piperidinyl, or azepanyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, R^{Y} is aziridinyl, azetidinyl, pyrrolidinyl, piperidinyl, or azepanyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, R^{Y} is unsubstituted aziridinyl, unsubstituted azetidinyl, unsubstituted pyrrolidinyl, unsubstituted piperidinyl, or unsubstituted azepanyl. In certain embodiments, R, is unsubstituted azetidinyl, unsubstituted pyrrolidinyl, or unsubstituted piperidinyl.

In certain embodiments, Ring B is of formula

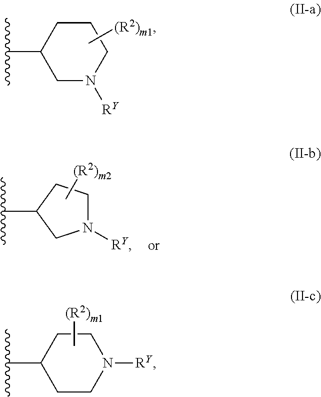

and R^{Y} is optionally substituted heterocyclyl containing a single nitrogen atom in the heterocyclyl. In certain embodiments, Ring B is of formula

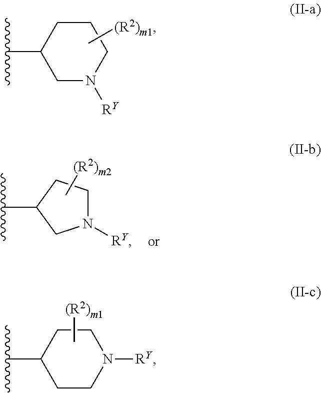

and R^{Y} is optionally substituted 3-14 membered heterocyclyl containing a single nitrogen atom in the heterocyclyl. In certain embodiments, Ring B is of formula

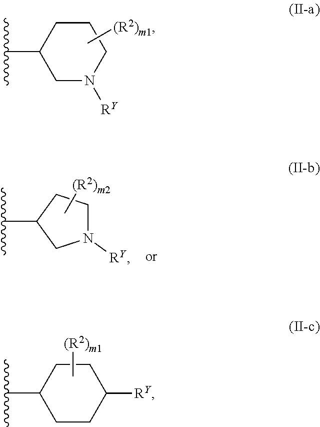

and R^{Y} is optionally substituted aziridinyl, optionally substituted azetidinyl, optionally substituted pyrrolidinyl, optionally substituted piperidinyl, or optionally substituted azepanyl. In certain embodiments, Ring B is of formula

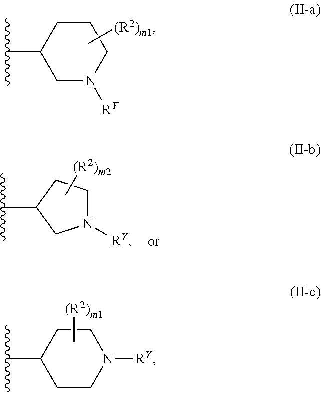

and R^{Y} is optionally substituted azetidinyl, optionally substituted pyrrolidinyl, or optionally substituted piperidinyl. In certain embodiments, Ring B is of formula

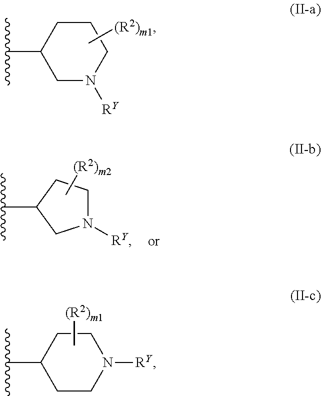

and R^{Y} is heterocyclyl containing a single nitrogen atom in the heterocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

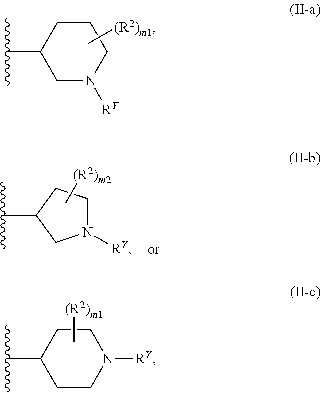

and R^{Y} is aziridinyl, azetidinyl, pyrrolidinyl, piperidinyl, or azepanyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

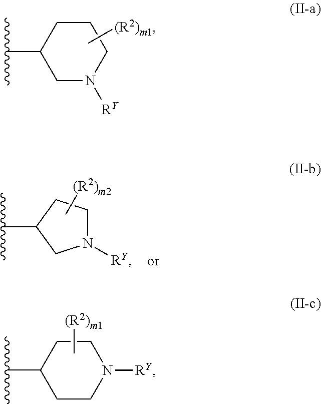

and R^{Y} is aziridinyl, azetidinyl, pyrrolidinyl, piperidinyl, or azepanyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

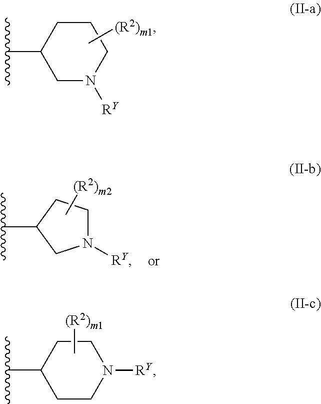

and R^{Y} is unsubstituted aziridinyl, unsubstituted azetidinyl, unsubstituted pyrrolidinyl, unsubstituted piperidinyl, or unsubstituted azepanyl. In certain embodiments, Ring B is of formula

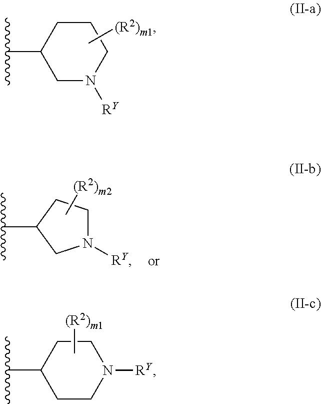

and R^{Y} is unsubstituted azetidinyl, unsubstituted pyrrolidinyl, or unsubstituted piperidinyl. In certain embodiments, Ring B is of formula

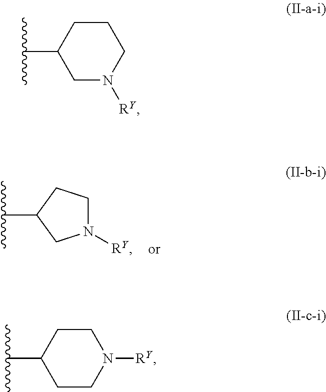

and R^{Y} is optionally substituted heterocyclyl containing a single nitrogen atom in the heterocyclyl. In certain embodiments, Ring B is of formula

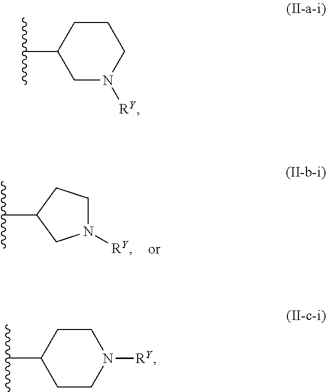

and R^{Y} is optionally substituted 3-14 membered heterocyclyl containing a single nitrogen atom in the heterocyclyl. In certain embodiments, Ring B is of formula

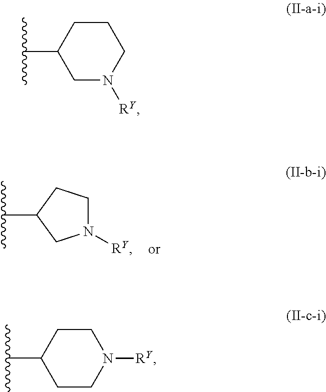

and R^{Y} is optionally substituted aziridinyl, optionally substituted azetidinyl, optionally substituted pyrrolidinyl, optionally substituted piperidinyl, or optionally substituted azepanyl. In certain embodiments, Ring B is of formula

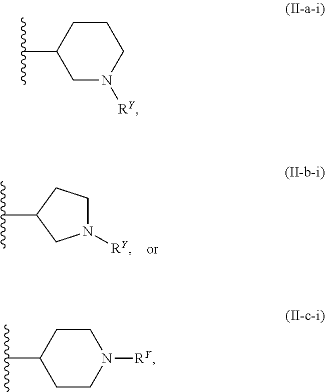

and R^{Y} is optionally substituted azetidinyl, optionally substituted pyrrolidinyl, or optionally substituted piperidinyl. In certain embodiments, Ring B is of formula

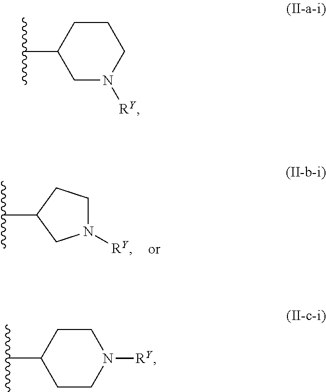

and R^{Y} is heterocyclyl containing a single nitrogen atom in the heterocyclyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

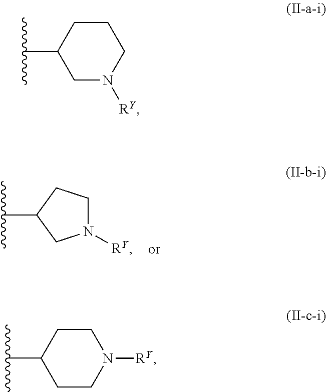

and R^{Y} is aziridinyl, azetidinyl, pyrrolidinyl, piperidinyl, or azepanyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A} —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

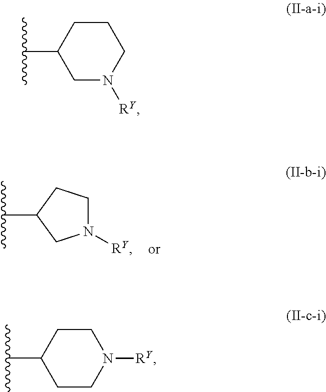

and R^{Y} is aziridinyl, azetidinyl, pyrrolidinyl, piperidinyl, or azepanyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

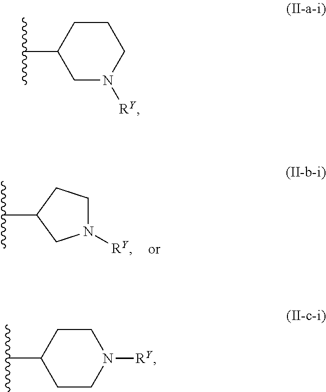

and R^{Y} is unsubstituted aziridinyl, unsubstituted azetidinyl, unsubstituted pyrrolidinyl, unsubstituted piperidinyl, or unsubstituted azepanyl. In certain embodiments, Ring B is of formula

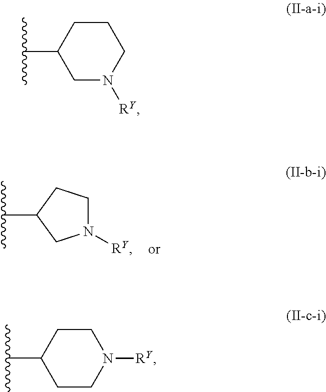

and R^{Y} is unsubstituted azetidinyl, unsubstituted pyrrolidinyl, or unsubstituted piperidinyl.

In certain embodiments, R^{Y} is optionally substituted aryl or optionally substituted heteroaryl. In certain embodiments, R^{Y} is optionally substituted aryl. In certain embodiments, R^{Y} is optionally substituted heteroaryl. In certain embodiments, R^{Y} is optionally substituted C₆₋₁₄ aryl or 5-14 membered heteroaryl. In certain embodiments, R^{Y} is optionally substituted phenyl. In certain embodiments, R^{Y} is substituted phenyl. In certain embodiments, R^{Y} is phenyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, R^{Y} is phenyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, R^{Y} is unsubstituted phenyl. In certain embodiments, R^{Y} is optionally substituted monocyclic heteroaryl. In certain embodiments, R^{Y} is optionally substituted 5- to 6-membered, monocyclic heteroaryl. In certain embodiments, R^{Y} is optionally substituted pyrrolyl, optionally substituted furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, or optionally substituted tetrazolyl. In certain embodiments, R^{Y} is optionally substituted pyridinyl, optionally substituted pyridazinyl, optionally substituted pyrimidinyl, optionally substituted pyrazinyl, optionally substituted triazinyl, optionally substituted tetrazinyl, optionally substituted oxepinyl, or optionally substituted thiepinyl. In certain embodiments, R^{Y} is optionally substituted bicyclic heteroaryl (e.g. optionally substituted bicyclic, 9- or 10-membered heteroaryl, wherein 1, 2, 3, or 4 atoms in the heteroaryl ring system are independently oxygen, nitrogen, or sulfur). In certain embodiments, R^{Y} is optionally substituted indolyl, optionally substituted isoindolyl, optionally substituted indazolyl, optionally substituted benzotriazolyl, optionally substituted benzothiophenyl, optionally substituted isobenzothiophenyl, optionally substituted benzofuranyl, optionally substituted benzoisofuranyl, optionally substituted benzimidazolyl, optionally substituted benzoxazolyl, optionally substituted benzisoxazolyl, optionally substituted benzoxadiazolyl, optionally substituted benzthiazolyl, optionally substituted benzisothiazolyl, optionally substituted benzthiadiazolyl, optionally substituted indolizinyl, optionally substituted purinyl. In certain embodiments, R^{Y} is optionally substituted naphthyridinyl, optionally substituted pteridinyl, optionally substituted quinolinyl, optionally substituted isoquinolinyl, optionally substituted cinnolinyl, optionally substituted quinoxalinyl, optionally substituted phthalazinyl, or optionally substituted quinazolinyl. In certain embodiments, R^{Y} is optionally substituted pyridinyl, optionally substituted pyrimidinyl, optionally substituted pyrazolyl, or optionally substituted imidazolyl. In certain embodiments, R^{Y} is heteroaryl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, R¹ is heteroaryl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, R^{Y} is pyridinyl, pyrimidinyl, pyrazolyl, or imidazolyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, R^{Y} is pyridinyl, pyrimidinyl, pyrazolyl, or imidazolyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, R, is optionally substituted phenyl, optionally substituted pyridinyl, optionally substituted pyrimidinyl, optionally substituted pyrazolyl, or optionally substituted imidazolyl.

In certain embodiments, Ring B is of formula

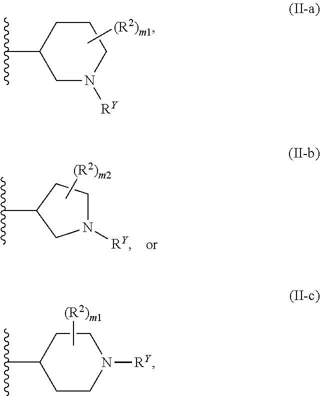

and R^{Y} is optionally substituted aryl. In certain embodiments, Ring B is of formula

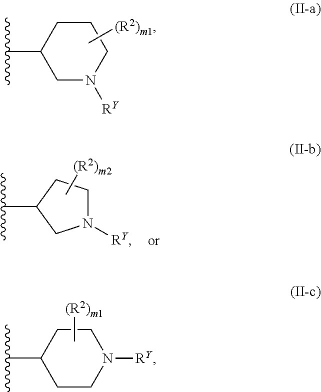

and R^{Y} is optionally substituted heteroaryl. In certain embodiments, Ring B is of formula

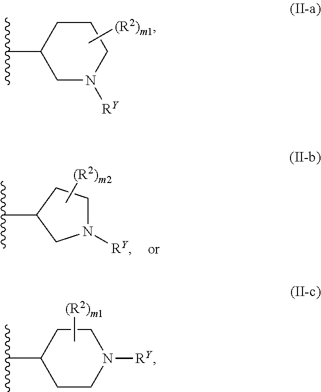

and R^{Y} is optionally substituted C₆₋₁₄ aryl or 5-14 membered heteroaryl. In certain embodiments, Ring B is of formula

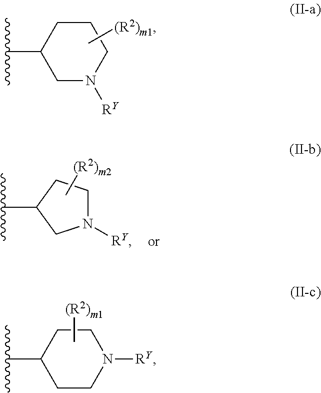

and R^{Y} is optionally substituted phenyl. In certain embodiments, Ring B is of formula

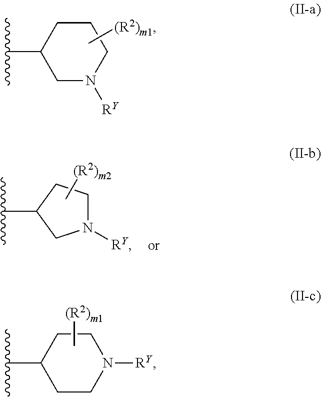

and R^{Y} is substituted phenyl. In certain embodiments, Ring B is of formula

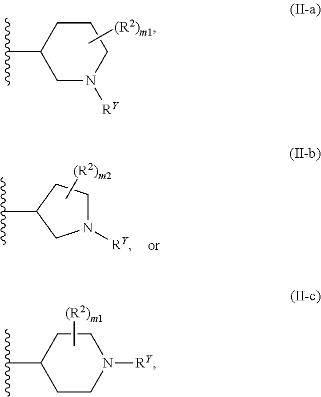

and R^{Y} is phenyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A} —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

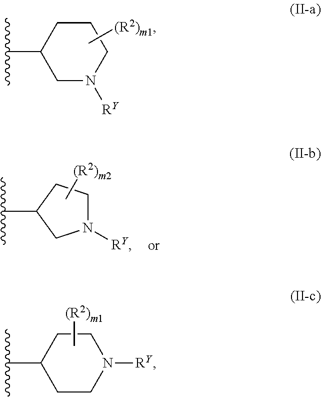

and R^{Y} is phenyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

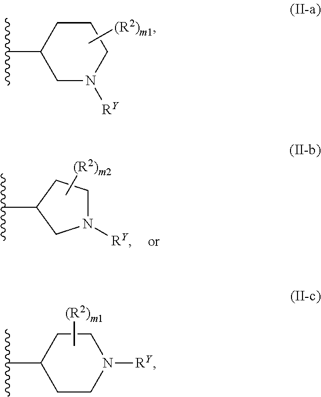

and R^{Y} is unsubstituted phenyl. In certain embodiments, Ring B is of formula

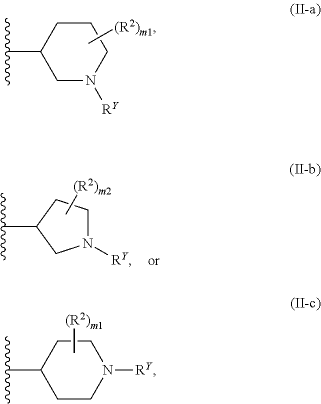

and R^{Y} is optionally substituted monocyclic heteroaryl. In certain embodiments, Ring B is of formula

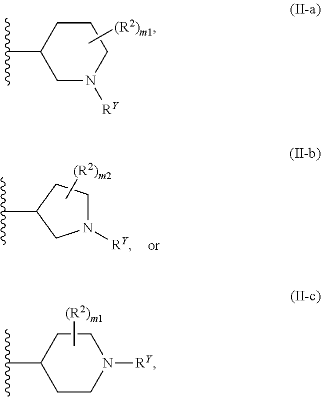

and R^{Y} is optionally substituted pyrrolyl, optionally substituted furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, or optionally substituted tetrazolyl. In certain embodiments, Ring B is of formula

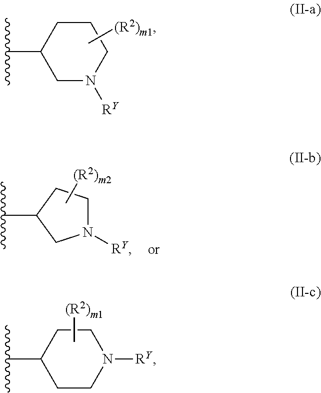

and R^{Y} is optionally substituted pyridinyl, optionally substituted pyridazinyl, optionally substituted pyrimidinyl, optionally substituted pyrazinyl, optionally substituted triazinyl, optionally substituted tetrazinyl, optionally substituted oxepinyl, or optionally substituted thiepinyl. In certain embodiments, Ring B is of formula

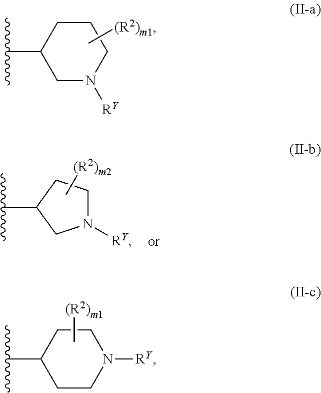

and R^{Y} is optionally substituted bicyclic heteroaryl (e.g. optionally substituted bicyclic, 9- or 10-membered heteroaryl, wherein 1, 2, 3, or 4 atoms in the heteroaryl ring system are independently oxygen, nitrogen, or sulfur). In certain embodiments, Ring B is of formula

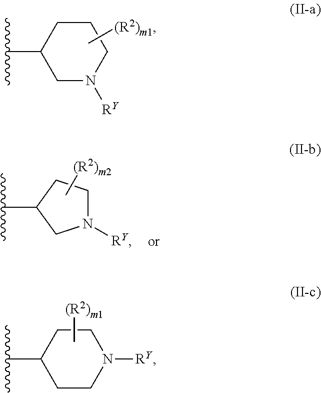

and R^{Y} is heteroaryl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

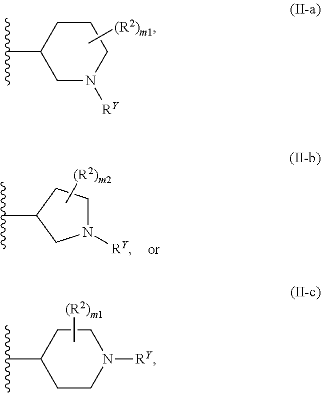

and R^{Y} is heteroaryl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

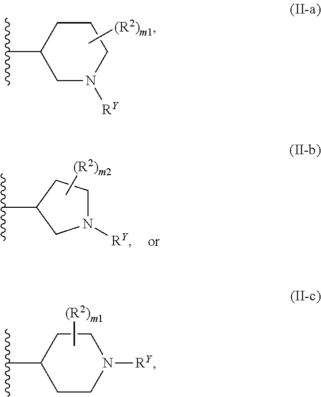

and R^{Y} is pyridinyl, pyrimidinyl, pyrazolyl, or imidazolyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

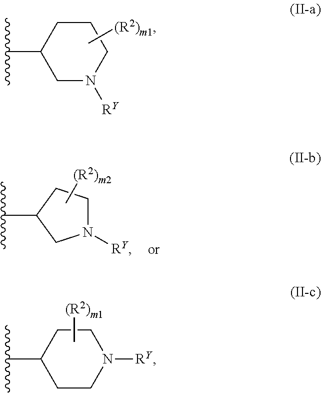

and R^{Y} is pyridinyl, pyrimidinyl, pyrazolyl, or imidazolyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

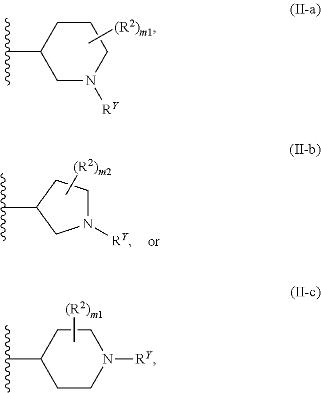

and R^{Y} is optionally substituted phenyl, optionally substituted pyridinyl, optionally substituted pyrimidinyl, optionally substituted pyrazolyl, or optionally substituted imidazolyl. In certain embodiments, Ring B is of formula

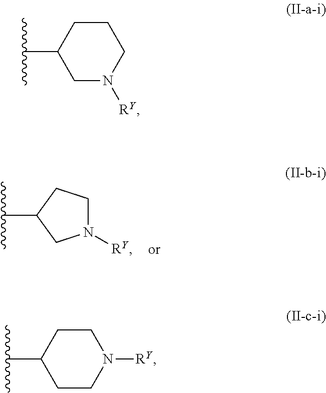

and R^{Y} is optionally substituted aryl. In certain embodiments, Ring B is of formula

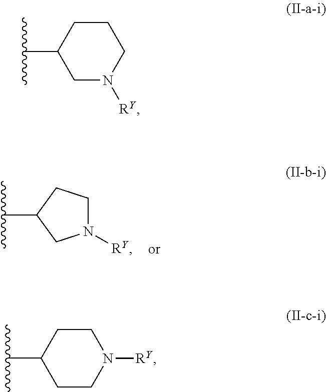

and R^{Y} is optionally substituted heteroaryl. In certain embodiments, Ring B is of formula

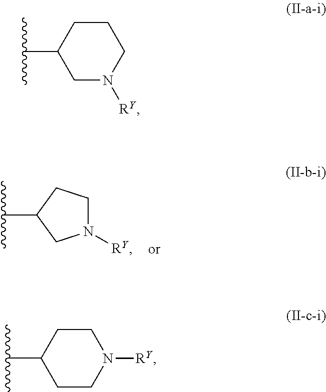

and R^{Y} is optionally substituted C₆₋₁₄ aryl or 5-14 membered heteroaryl. In certain embodiments, Ring B is of formula

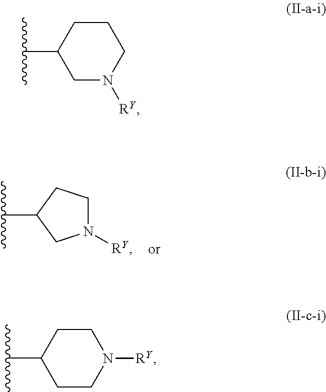

and R^{Y} is optionally substituted phenyl. In certain embodiments, Ring B is of formula

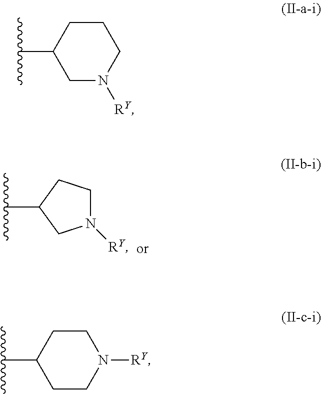

and R^{Y} is substituted phenyl. In certain embodiments, Ring B is of formula

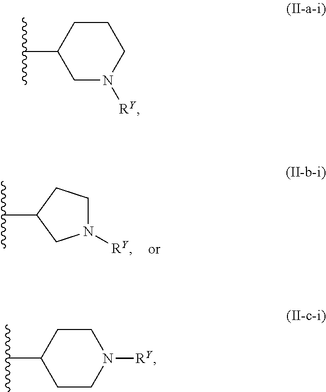

and R^{Y} is phenyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A} —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

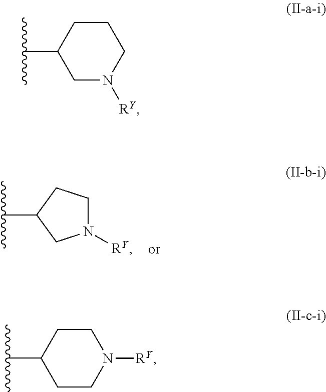

and R^{Y} is phenyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

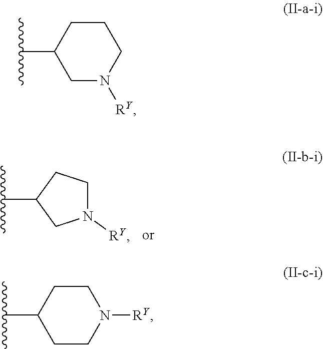

and R^{Y} is unsubstituted phenyl. In certain embodiments, Ring B is of formula

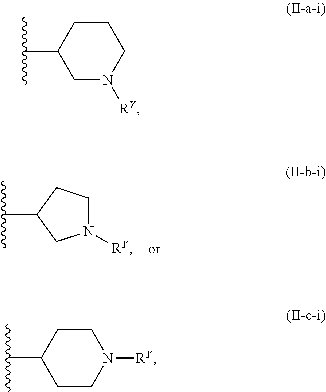

and R^{Y} is optionally substituted monocyclic heteroaryl. In certain embodiments, Ring B is of formula

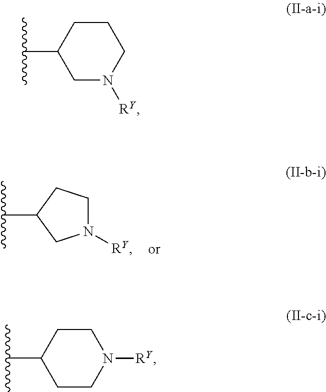

and R^{Y} is optionally substituted pyrrolyl, optionally substituted furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, or optionally substituted tetrazolyl. In certain embodiments, Ring B is of formula

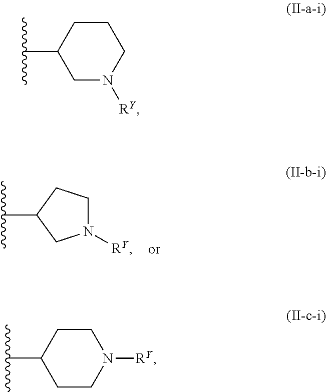

and R^{Y} is optionally substituted pyridinyl, optionally substituted pyridazinyl, optionally substituted pyrimidinyl, optionally substituted pyrazinyl, optionally substituted triazinyl, optionally substituted tetrazinyl, optionally substituted oxepinyl, or optionally substituted thiepinyl. In certain embodiments, Ring B is of formula

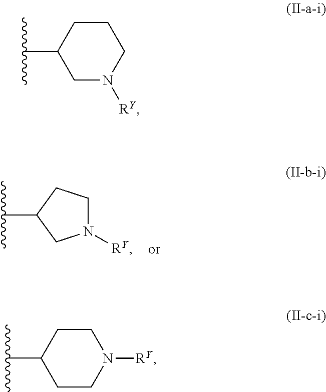

and R^{Y} is optionally substituted bicyclic heteroaryl (e.g. optionally substituted bicyclic, 9- or 10-membered heteroaryl, wherein 1, 2, 3, or 4 atoms in the heteroaryl ring system are independently oxygen, nitrogen, or sulfur). In certain embodiments, Ring B is of formula

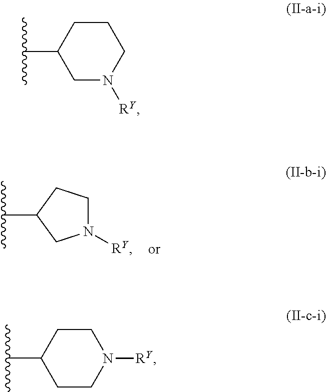

and R^{Y} is heteroaryl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

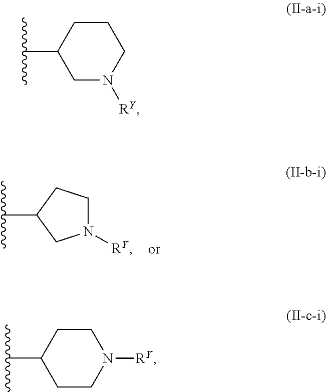

and R^{Y} is heteroaryl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

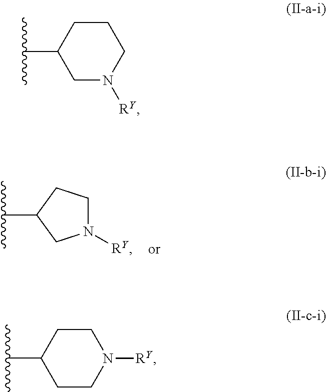

and R^{Y} is pyridinyl, pyrimidinyl, pyrazolyl, or imidazolyl optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A} —NR^{A}C(═O)OR^{A} —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, Ring B is of formula

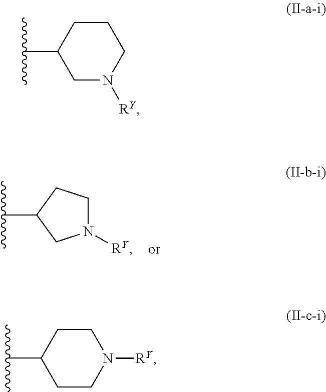

and R^{Y} is pyridinyl, pyrimidinyl, pyrazolyl, or imidazolyl optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, Ring B is of formula

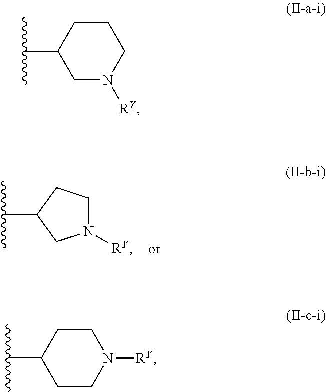

and R^{Y} is optionally substituted phenyl, optionally substituted pyridinyl, optionally substituted pyrimidinyl, optionally substituted pyrazolyl, or optionally substituted imidazolyl.

In certain embodiments, R^{Y} is optionally substituted alkyl substituted with optionally substituted heteroaryl, or optionally substituted alkyl substituted with optionally substituted heterocyclyl comprising at least one ring oxo or thiooxo substituent in the heterocyclyl. In certain embodiments, R^{Y} is optionally substituted alkyl substituted with optionally substituted heteroaryl. In certain embodiments, R^{Y} is optionally substituted C₁₋₁₀ alkyl substituted with optionally substituted heteroaryl. In certain embodiments, R^{Y} is optionally substituted C₁₋₆ alkyl substituted with optionally substituted heteroaryl. In certain embodiments, R^{Y} is optionally substituted methyl substituted with optionally substituted heteroaryl, optionally substituted ethyl substituted with optionally substituted heteroaryl, optionally substituted n-propyl substituted with optionally substituted heteroaryl, optionally substituted isopropyl substituted with optionally substituted heteroaryl, optionally substituted n-butyl substituted with optionally substituted heteroaryl, optionally substituted tert-butyl substituted with optionally substituted heteroaryl, optionally substituted sec-butyl substituted with optionally substituted heteroaryl, optionally substituted isobutyl substituted with optionally substituted heteroaryl, optionally substituted n-pentyl substituted with optionally substituted heteroaryl, optionally substituted 3-pentanyl substituted with optionally substituted heteroaryl, optionally substituted amyl substituted with optionally substituted heteroaryl, optionally substituted neopentyl substituted with optionally substituted heteroaryl, optionally substituted 3-methyl-2-butanyl substituted with optionally substituted heteroaryl, optionally substituted tert-amyl substituted with optionally substituted heteroaryl, or optionally substituted n-hexyl substituted with optionally substituted heteroaryl. In certain embodiments, R^{Y} is optionally substituted methyl substituted with optionally substituted heteroaryl, optionally substituted ethyl substituted with optionally substituted heteroaryl, or optionally substituted n-propyl substituted with optionally substituted heteroaryl. In certain embodiments, the optionally substituted heteroaryl is substituted. In certain embodiments, the optionally substituted heteroaryl is optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, the optionally substituted heteroaryl is optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, the optionally substituted heteroaryl is unsubstituted. In certain embodiments, the optionally substituted heteroaryl is optionally substituted pyrazolyl or optionally substituted imidazolyl.

In certain embodiments, Ring B is of formula

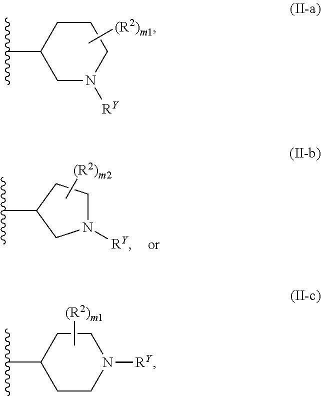

and R^{Y} is optionally substituted alkyl substituted with optionally substituted heteroaryl. In certain embodiments, Ring B is of formula

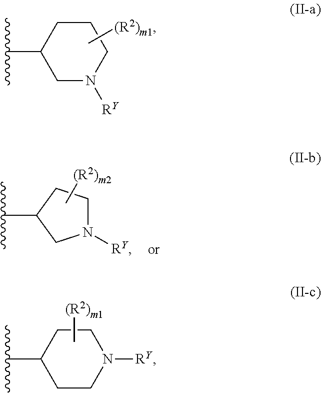

and R^{Y} is optionally substituted C₁₋₆ alkyl substituted with optionally substituted heteroaryl. In certain embodiments, Ring B is of formula

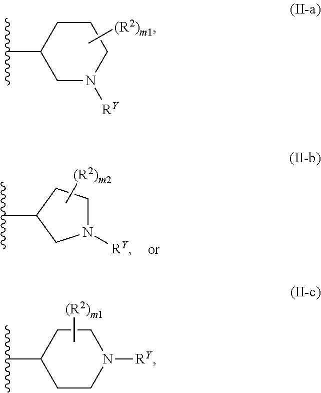

and R^{Y} is optionally substituted methyl substituted with optionally substituted heteroaryl, optionally substituted ethyl substituted with optionally substituted heteroaryl, optionally substituted n-propyl substituted with optionally substituted heteroaryl, optionally substituted isopropyl substituted with optionally substituted heteroaryl, optionally substituted n-butyl substituted with optionally substituted heteroaryl, optionally substituted tert-butyl substituted with optionally substituted heteroaryl, optionally substituted sec-butyl substituted with optionally substituted heteroaryl, optionally substituted isobutyl substituted with optionally substituted heteroaryl, optionally substituted n-pentyl substituted with optionally substituted heteroaryl, optionally substituted 3-pentanyl substituted with optionally substituted heteroaryl, optionally substituted amyl substituted with optionally substituted heteroaryl, optionally substituted neopentyl substituted with optionally substituted heteroaryl, optionally substituted 3-methyl-2-butanyl substituted with optionally substituted heteroaryl, optionally substituted tert-amyl substituted with optionally substituted heteroaryl, or optionally substituted n-hexyl substituted with optionally substituted heteroaryl. In certain embodiments, Ring B is of formula

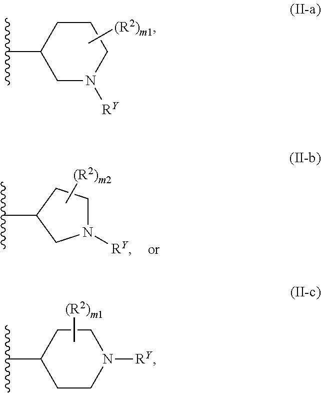

and R^{Y} is optionally substituted methyl substituted with optionally substituted heteroaryl, optionally substituted ethyl substituted with optionally substituted heteroaryl, or optionally substituted n-propyl substituted with optionally substituted heteroaryl. In certain embodiments, the optionally substituted heteroaryl is substituted. In certain embodiments, the optionally substituted heteroaryl is optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, the optionally substituted heteroaryl is optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, the optionally substituted heteroaryl is unsubstituted. In certain embodiments, the optionally substituted heteroaryl is optionally substituted pyrazolyl or optionally substituted imidazolyl. In certain embodiments, Ring B is of formula

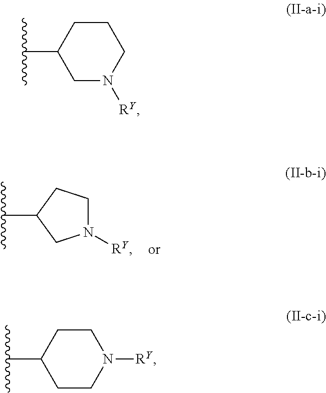

and R^{Y} is optionally substituted alkyl substituted with optionally substituted heteroaryl. In certain embodiments, Ring B is of formula

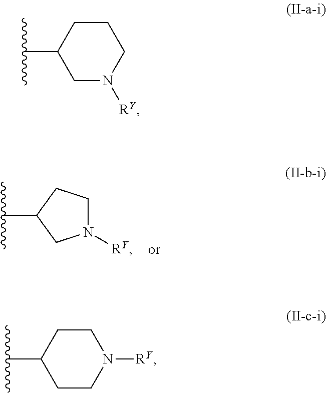

and R^{Y} is optionally substituted C₁₋₆ alkyl substituted with optionally substituted heteroaryl. In certain embodiments, Ring B is of formula

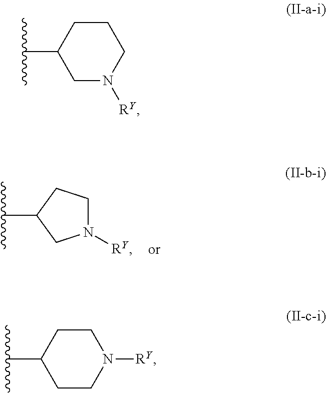

and R^{Y} is optionally substituted methyl substituted with optionally substituted heteroaryl, optionally substituted ethyl substituted with optionally substituted heteroaryl, optionally substituted n-propyl substituted with optionally substituted heteroaryl, optionally substituted isopropyl substituted with optionally substituted heteroaryl, optionally substituted n-butyl substituted with optionally substituted heteroaryl, optionally substituted tert-butyl substituted with optionally substituted heteroaryl, optionally substituted sec-butyl substituted with optionally substituted heteroaryl, optionally substituted isobutyl substituted with optionally substituted heteroaryl, optionally substituted n-pentyl substituted with optionally substituted heteroaryl, optionally substituted 3-pentanyl substituted with optionally substituted heteroaryl, optionally substituted amyl substituted with optionally substituted heteroaryl, optionally substituted neopentyl substituted with optionally substituted heteroaryl, optionally substituted 3-methyl-2-butanyl substituted with optionally substituted heteroaryl, optionally substituted tert-amyl substituted with optionally substituted heteroaryl, or optionally substituted n-hexyl substituted with optionally substituted heteroaryl. In certain embodiments, Ring B is of formula

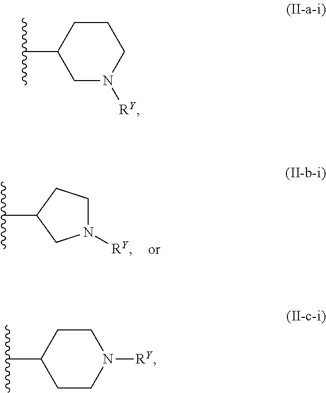

and R^{Y} is optionally substituted methyl substituted with optionally substituted heteroaryl, optionally substituted ethyl substituted with optionally substituted heteroaryl, or optionally substituted n-propyl substituted with optionally substituted heteroaryl. In certain embodiments, the optionally substituted heteroaryl is substituted. In certain embodiments, the optionally substituted heteroaryl is optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, the optionally substituted heteroaryl is optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, the optionally substituted heteroaryl is unsubstituted. In certain embodiments, the optionally substituted heteroaryl is optionally substituted pyrazolyl or optionally substituted imidazolyl.

In certain embodiments, R^{Y} is optionally substituted alkyl substituted with optionally substituted heterocyclyl comprising at least one ring oxo or thiooxo substituent in the heterocyclyl. In certain embodiments, R^{Y} is optionally substituted C₁₋₁₀ alkyl substituted with optionally substituted heterocyclyl comprising at least one ring oxo or thiooxo substituent in the heterocyclyl. In certain embodiments, R is optionally substituted C₁₋₆ alkyl substituted with optionally substituted heterocyclyl comprising at least one ring oxo or thiooxo substituent in the heterocyclyl. In certain embodiments, R, is optionally substituted methyl substituted with optionally substituted heterocyclyl comprising at least one ring oxo or thiooxo substituent in the heterocyclyl, optionally substituted ethyl substituted with optionally substituted heterocyclyl comprising at least one ring oxo or thiooxo substituent in the heterocyclyl, optionally substituted n-propyl substituted with optionally substituted heterocyclyl comprising at least one ring oxo or thiooxo substituent in the heterocyclyl, optionally substituted isopropyl substituted with optionally substituted heterocyclyl comprising at least one ring oxo or thiooxo substituent in the heterocyclyl, optionally substituted n-butyl substituted with optionally substituted heterocyclyl comprising at least one ring oxo or thiooxo substituent in the heterocyclyl, optionally substituted tert-butyl substituted with optionally substituted heterocyclyl comprising at least one ring oxo or thiooxo substituent in the heterocyclyl, optionally substituted sec-butyl substituted with optionally substituted heterocyclyl comprising at least one ring oxo or thiooxo substituent in the heterocyclyl, optionally substituted isobutyl substituted with optionally substituted heterocyclyl comprising at least one ring oxo or thiooxo substituent in the heterocyclyl, optionally substituted n-pentyl substituted with optionally substituted heterocyclyl comprising at least one ring oxo or thiooxo substituent in the heterocyclyl, optionally substituted 3-pentanyl substituted with optionally substituted heterocyclyl comprising at least one ring oxo or thiooxo substituent in the heterocyclyl, optionally substituted amyl substituted with optionally substituted heterocyclyl comprising at least one ring oxo or thiooxo substituent in the heterocyclyl, optionally substituted neopentyl substituted with optionally substituted heterocyclyl comprising at least one ring oxo or thiooxo substituent in the heterocyclyl, optionally substituted 3-methyl-2-butanyl substituted with optionally substituted heterocyclyl comprising at least one ring oxo or thiooxo substituent in the heterocyclyl, optionally substituted tert-amyl substituted with optionally substituted heterocyclyl comprising at least one ring oxo or thiooxo substituent in the heterocyclyl, or optionally substituted n-hexyl substituted with optionally substituted heterocyclyl comprising at least one ring oxo or thiooxo substituent in the heterocyclyl. In certain embodiments, R^{Y} is optionally substituted methyl substituted with optionally substituted heterocyclyl comprising at least one ring oxo or thiooxo substituent in the heterocyclyl, optionally substituted ethyl substituted with optionally substituted heterocyclyl comprising at least one ring oxo or thiooxo substituent in the heterocyclyl, or optionally substituted n-propyl substituted with optionally substituted heterocyclyl comprising at least one ring oxo or thiooxo substituent in the heterocyclyl. In certain embodiments, the heterocyclyl comprising at least one ring oxo or thiooxo substituent in the heterocyclyl is substituted. In certain embodiments, the heterocyclyl comprising at least one ring oxo or thiooxo substituent in the heterocyclyl is optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, the heterocyclyl comprising at least one ring oxo or thiooxo substituent in the heterocyclyl is optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, the heterocyclyl comprising at least one ring oxo or thiooxo substituent in the heterocyclyl is optionally substituted 3-14 membered heterocyclyl. In certain embodiments, the heterocyclyl comprising at least one ring oxo or thiooxo substituent in the heterocyclyl is substituted or unsubstituted, monocyclic heterocyclyl (e.g., substituted or unsubstituted, 3- to 7-membered, monocyclic heterocyclyl). In certain embodiments, the heterocyclyl comprising at least one ring oxo or thiooxo substituent in the heterocyclyl optionally substituted oxazolidinonyl or optionally substituted pyrrolidinonyl. In certain embodiments, the heterocyclyl comprising at least one ring oxo or thiooxo substituent in the heterocyclyl is substituted oxazolidinonyl or substituted pyrrolidinonyl. In certain embodiments, the heterocyclyl comprising at least one ring oxo or thiooxo substituent in the heterocyclyl is unsubstituted oxazolidinonyl or unsubstituted pyrrolidinonyl.

In certain embodiments, Ring B is of formula

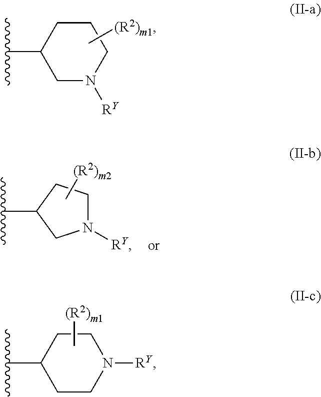

and R^{Y} is optionally substituted alkyl substituted with optionally substituted heterocyclyl comprising at least one ring oxo or thiooxo substituent in the heterocyclyl. In certain embodiments, Ring B is of formula

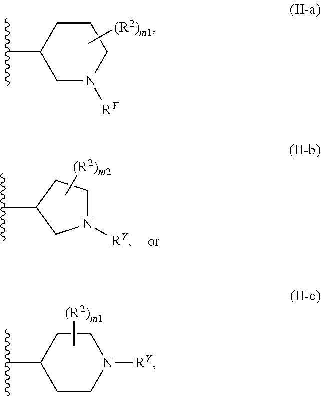

and R^{Y} is optionally substituted C₁₋₆ alkyl substituted with optionally substituted heterocyclyl comprising at least one ring oxo or thiooxo substituent in the heterocyclyl. In certain embodiments, Ring B is of formula

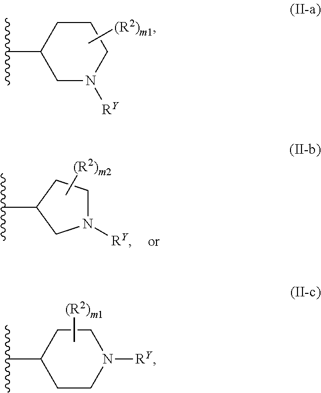

and R^{Y} is optionally substituted methyl substituted with optionally substituted heterocyclyl comprising at least one ring oxo or thiooxo substituent in the heterocyclyl, optionally substituted ethyl substituted with optionally substituted heterocyclyl comprising at least one ring oxo or thiooxo substituent in the heterocyclyl, optionally substituted n-propyl substituted with optionally substituted heterocyclyl comprising at least one ring oxo or thiooxo substituent in the heterocyclyl, optionally substituted isopropyl substituted with optionally substituted heterocyclyl comprising at least one ring oxo or thiooxo substituent in the heterocyclyl, optionally substituted n-butyl substituted with optionally substituted heterocyclyl comprising at least one ring oxo or thiooxo substituent in the heterocyclyl, optionally substituted tert-butyl substituted with optionally substituted heterocyclyl comprising at least one ring oxo or thiooxo substituent in the heterocyclyl, optionally substituted sec-butyl substituted with optionally substituted heterocyclyl comprising at least one ring oxo or thiooxo substituent in the heterocyclyl, optionally substituted isobutyl substituted with optionally substituted heterocyclyl comprising at least one ring oxo or thiooxo substituent in the heterocyclyl, optionally substituted n-pentyl substituted with optionally substituted heterocyclyl comprising at least one ring oxo or thiooxo substituent in the heterocyclyl, optionally substituted 3-pentanyl substituted with optionally substituted heterocyclyl comprising at least one ring oxo or thiooxo substituent in the heterocyclyl, optionally substituted amyl substituted with optionally substituted heterocyclyl comprising at least one ring oxo or thiooxo substituent in the heterocyclyl, optionally substituted neopentyl substituted with optionally substituted heterocyclyl comprising at least one ring oxo or thiooxo substituent in the heterocyclyl, optionally substituted 3-methyl-2-butanyl substituted with optionally substituted heterocyclyl comprising at least one ring oxo or thiooxo substituent in the heterocyclyl, optionally substituted tert-amyl substituted with optionally substituted heterocyclyl comprising at least one ring oxo or thiooxo substituent in the heterocyclyl, or optionally substituted n-hexyl substituted with optionally substituted heterocyclyl comprising at least one ring oxo or thiooxo substituent in the heterocyclyl. In certain embodiments, Ring B is of formula

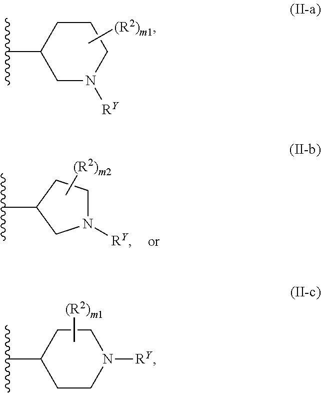

and R^{Y} is optionally substituted methyl substituted with optionally substituted heterocyclyl comprising at least one ring oxo or thiooxo substituent in the heterocyclyl, optionally substituted ethyl substituted with optionally substituted heterocyclyl comprising at least one ring oxo or thiooxo substituent in the heterocyclyl, or optionally substituted n-propyl substituted with optionally substituted heterocyclyl comprising at least one ring oxo or thiooxo substituent in the heterocyclyl. In certain embodiments, the heterocyclyl comprising at least one ring oxo or thiooxo substituent in the heterocyclyl is substituted. In certain embodiments, the heterocyclyl comprising at least one ring oxo or thiooxo substituent in the heterocyclyl is optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A} —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, the heterocyclyl comprising at least one ring oxo or thiooxo substituent in the heterocyclyl is optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, the heterocyclyl comprising at least one ring oxo or thiooxo substituent in the heterocyclyl is optionally substituted 3-14 membered heterocyclyl. In certain embodiments, the heterocyclyl comprising at least one ring oxo or thiooxo substituent in the heterocyclyl is substituted or unsubstituted, monocyclic heterocyclyl (e.g., substituted or unsubstituted, 3- to 7-membered, monocyclic heterocyclyl). In certain embodiments, the heterocyclyl comprising at least one ring oxo or thiooxo substituent in the heterocyclyl optionally substituted oxazolidinonyl or optionally substituted pyrrolidinonyl. In certain embodiments, the heterocyclyl comprising at least one ring oxo or thiooxo substituent in the heterocyclyl is substituted oxazolidinonyl or substituted pyrrolidinonyl. In certain embodiments, the heterocyclyl comprising at least one ring oxo or thiooxo substituent in the heterocyclyl is unsubstituted oxazolidinonyl or unsubstituted pyrrolidinonyl. In certain embodiments, Ring B is of formula

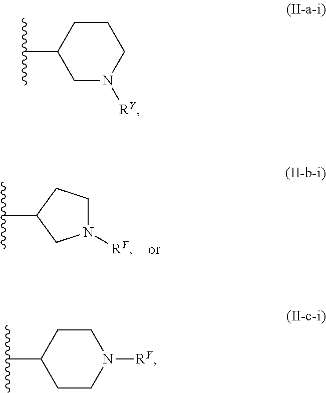

and R^{Y} is optionally substituted alkyl substituted with optionally substituted heterocyclyl comprising at least one ring oxo or thiooxo substituent in the heterocyclyl. In certain embodiments, Ring B is of formula

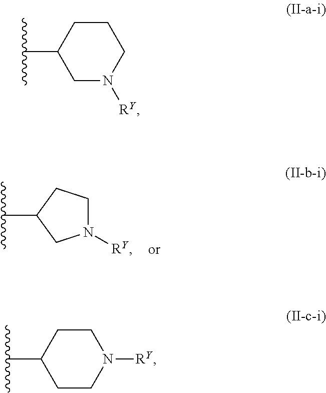

and R^{Y} is optionally substituted C₁₋₆ alkyl substituted with optionally substituted heterocyclyl comprising at least one ring oxo or thiooxo substituent in the heterocyclyl. In certain embodiments, Ring B is of formula

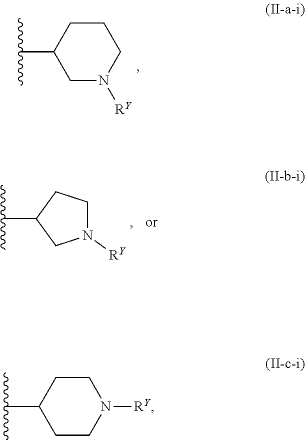

and R^{Y} is optionally substituted methyl substituted with optionally substituted heterocyclyl comprising at least one ring oxo or thiooxo substituent in the heterocyclyl, optionally substituted ethyl substituted with optionally substituted heterocyclyl comprising at least one ring oxo or thiooxo substituent in the heterocyclyl, optionally substituted n-propyl substituted with optionally substituted heterocyclyl comprising at least one ring oxo or thiooxo substituent in the heterocyclyl, optionally substituted isopropyl substituted with optionally substituted heterocyclyl comprising at least one ring oxo or thiooxo substituent in the heterocyclyl, optionally substituted n-butyl substituted with optionally substituted heterocyclyl comprising at least one ring oxo or thiooxo substituent in the heterocyclyl, optionally substituted tert-butyl substituted with optionally substituted heterocyclyl comprising at least one ring oxo or thiooxo substituent in the heterocyclyl, optionally substituted sec-butyl substituted with optionally substituted heterocyclyl comprising at least one ring oxo or thiooxo substituent in the heterocyclyl, optionally substituted isobutyl substituted with optionally substituted heterocyclyl comprising at least one ring oxo or thiooxo substituent in the heterocyclyl, optionally substituted n-pentyl substituted with optionally substituted heterocyclyl comprising at least one ring oxo or thiooxo substituent in the heterocyclyl, optionally substituted 3-pentanyl substituted with optionally substituted heterocyclyl comprising at least one ring oxo or thiooxo substituent in the heterocyclyl, optionally substituted amyl substituted with optionally substituted heterocyclyl comprising at least one ring oxo or thiooxo substituent in the heterocyclyl, optionally substituted neopentyl substituted with optionally substituted heterocyclyl comprising at least one ring oxo or thiooxo substituent in the heterocyclyl, optionally substituted 3-methyl-2-butanyl substituted with optionally substituted heterocyclyl comprising at least one ring oxo or thiooxo substituent in the heterocyclyl, optionally substituted tert-amyl substituted with optionally substituted heterocyclyl comprising at least one ring oxo or thiooxo substituent in the heterocyclyl, or optionally substituted n-hexyl substituted with optionally substituted heterocyclyl comprising at least one ring oxo or thiooxo substituent in the heterocyclyl. In certain embodiments, Ring B is of formula

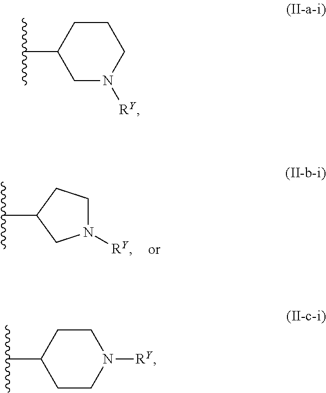

and R^{Y} is optionally substituted methyl substituted with optionally substituted heterocyclyl comprising at least one ring oxo or thiooxo substituent in the heterocyclyl, optionally substituted ethyl substituted with optionally substituted heterocyclyl comprising at least one ring oxo or thiooxo substituent in the heterocyclyl, or optionally substituted n-propyl substituted with optionally substituted heterocyclyl comprising at least one ring oxo or thiooxo substituent in the heterocyclyl. In certain embodiments, the heterocyclyl comprising at least one ring oxo or thiooxo substituent in the heterocyclyl is substituted. In certain embodiments, the heterocyclyl comprising at least one ring oxo or thiooxo substituent in the heterocyclyl is optionally substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, the heterocyclyl comprising at least one ring oxo or thiooxo substituent in the heterocyclyl is optionally substituted with halogen, optionally substituted alkyl, optionally substituted alkenyl, optionally substituted heteroalkyl, optionally substituted heterocyclyl, optionally substituted heteroaryl, —C(═O)R^{A}, —C(═O)OR^{A}, and/or —N(R^{A})₂ groups. In certain embodiments, the heterocyclyl comprising at least one ring oxo or thiooxo substituent in the heterocyclyl is optionally substituted 3-14 membered heterocyclyl. In certain embodiments, the heterocyclyl comprising at least one ring oxo or thiooxo substituent in the heterocyclyl is substituted or unsubstituted, monocyclic heterocyclyl (e.g., substituted or unsubstituted, 3- to 7-membered, monocyclic heterocyclyl). In certain embodiments, the heterocyclyl comprising at least one ring oxo or thiooxo substituent in the heterocyclyl optionally substituted oxazolidinonyl or optionally substituted pyrrolidinonyl. In certain embodiments, the heterocyclyl comprising at least one ring oxo or thiooxo substituent in the heterocyclyl is substituted oxazolidinonyl or substituted pyrrolidinonyl. In certain embodiments, the heterocyclyl comprising at least one ring oxo or thiooxo substituent in the heterocyclyl is unsubstituted oxazolidinonyl or unsubstituted pyrrolidinonyl.

In certain embodiments, each occurrence of R² is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; or two occurrences of R² are taken together to form ═O or ═S, two occurrences of R² are taken together to form an optionally substituted carbocyclyl, or two occurrences of R² are taken together to form an optionally substituted heterocyclyl.

In certain embodiments, at least one occurrence of R² is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A} —NR^{A}S(═O)N(R^{A})₂, —NR^{A}S(═O)₂R^{A} —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, at least one occurrence of R² is halogen. In certain embodiments, at least one occurrence of R² is optionally substituted alkyl. In certain embodiments, at least one occurrence of R² is optionally substituted alkenyl. In certain embodiments, at least one occurrence of R² is optionally substituted alkynyl. In certain embodiments, at least one occurrence of R² is optionally substituted heteroalkyl. In certain embodiments, at least one occurrence of R² is optionally substituted heteroalkenyl. In certain embodiments, at least one occurrence of R² is optionally substituted heteroalkynyl. In certain embodiments, at least one occurrence of R² is optionally substituted carbocyclyl. In certain embodiments, at least one occurrence of R² is optionally substituted heterocyclyl. In certain embodiments, at least one occurrence of R² is optionally substituted aryl. In certain embodiments, at least one occurrence of R² is optionally substituted heteroaryl. In certain embodiments, at least one occurrence of R² is —CN. In certain embodiments, at least one occurrence of R² is —OR^{A}. In certain embodiments, at least one occurrence of R² is —SCN. In certain embodiments, at least one occurrence of R² is —SR^{A}. In certain embodiments, at least one occurrence of R² is —SSR^{A}. In certain embodiments, at least one occurrence of R² is —N₃. In certain embodiments, at least one occurrence of R² is —NO. In certain embodiments, at least one occurrence of R² is —N(R^{A})₂. In certain embodiments, at least one occurrence of R² is —NO₂. In certain embodiments, at least one occurrence of R² is —C(═O)R^{A}. In certain embodiments, at least one occurrence of R² is —C(═O)OR^{A}. In certain embodiments, at least one occurrence of R² is —C(═O)SR^{A}. In certain embodiments, at least one occurrence of R² is —C(═O)N(R^{A})₂. In certain embodiments, at least one occurrence of R² is —C(═NR^{A})R^{A}. In certain embodiments, at least one occurrence of R² is —C(═NR^{A})OR^{A}. In certain embodiments, at least one occurrence of R² is —C(═NR^{A})SR^{A}. In certain embodiments, at least one occurrence of R² is —C(═NR^{A})N(R^{A})₂. In certain embodiments, at least one occurrence of R² is —S(═O)R^{A}. In certain embodiments, at least one occurrence of R² is —S(═O)OR^{A}. In certain embodiments, at least one occurrence of R² is —S(═O)SR^{A}. In certain embodiments, at least one occurrence of R² is —S(═O)N(R^{A})₂. In certain embodiments, at least one occurrence of R² is —S(═O)₂R^{A}. In certain embodiments, at least one occurrence of R² is —S(═O)₂OR^{A}. In certain embodiments, at least one occurrence of R² is —S(═O)₂SR^{A}. In certain embodiments, at least one occurrence of R² is —S(═O)₂N(R^{A})₂. In certain embodiments, at least one occurrence of R² is —OC(═O)R^{A}. In certain embodiments, at least one occurrence of R² is —OC(═O)OR^{A}. In certain embodiments, at least one occurrence of R² is —OC(═O)SR^{A}. In certain embodiments, at least one occurrence of R² is —OC(═O)N(R^{A})₂. In certain embodiments, at least one occurrence of R² is —OC(═NR^{A})R^{A}. In certain embodiments, at least one occurrence of R² is —OC(═NR^{A})OR^{A}. In certain embodiments, at least one occurrence of R² is —OC(═NR^{A})SR^{A}. In certain embodiments, at least one occurrence of R² is —OC(═NR^{A})N(R^{A})₂. In certain embodiments, at least one occurrence of R² is —OS(═O)R^{A}. In certain embodiments, at least one occurrence of R² is —OS(═O)OR^{A}. In certain embodiments, at least one occurrence of R² is —OS(═O)SR^{A}. In certain embodiments, at least one occurrence of R² is —OS(═O)N(R^{A})₂. In certain embodiments, at least one occurrence of R² is —OS(═O)₂R^{A}. In certain embodiments, at least one occurrence of R² is —OS(═O)₂OR^{A}. In certain embodiments, at least one occurrence of R² is —OS(═O)₂SR^{A}. In certain embodiments, at least one occurrence of R² is —OS(═O)₂N(R^{A})₂. In certain embodiments, at least one occurrence of R² is —ON(R^{A})₂. In certain embodiments, at least one occurrence of R² is —SC(═O)R^{A}. In certain embodiments, at least one occurrence of R² is —SC(═O)OR^{A}. In certain embodiments, at least one occurrence of R² is —SC(═O)SR^{A}. In certain embodiments, at least one occurrence of R² is —SC(═O)N(R^{A})₂. In certain embodiments, at least one occurrence of R² is —SC(═NR^{A})R^{A}. In certain embodiments, at least one occurrence of R² is —SC(═NR^{A})OR^{A}. In certain embodiments, at least one occurrence of R² is —SC(═NR^{A})SR^{A}. In certain embodiments, at least one occurrence of R² is —SC(═NR^{A})N(R^{A})₂. In certain embodiments, at least one occurrence of R² is —NR^{A}C(═O)R^{A}. In certain embodiments, at least one occurrence of R² is —NR^{A}C(═O)OR^{A}. In certain embodiments, at least one occurrence of R² is —NR^{A}C(═O)SR^{A}. In certain embodiments, at least one occurrence of R² is —NR^{A}C(═O)N(R^{A})₂. In certain embodiments, at least one occurrence of R² is —NR^{A}C(═NR^{A})R^{A}. In certain embodiments, at least one occurrence of R² is —NR^{A}C(═NR^{A})OR^{A}. In certain embodiments, at least one occurrence of R² is —NR^{A}C(═NR^{A})SR^{A}. In certain embodiments, at least one occurrence of R² is —NR^{A}C(═NR^{A})N(R^{A})₂. In certain embodiments, at least one occurrence of R² is —NR^{A}S(═O)R^{A}. In certain embodiments, at least one occurrence of R² is —NR^{A}S(═O)OR^{A}. In certain embodiments, at least one occurrence of R² is —NR^{A}S(═O)SR^{A}. In certain embodiments, at least one occurrence of R² is —NR^{A}S(═O)N(R^{A})₂. In certain embodiments, at least one occurrence of R² is —NR^{A}S(═O)₂R^{A}. In certain embodiments, at least one occurrence of R² is —NR^{A}S(═O)₂OR^{A}. In certain embodiments, at least one occurrence of R² is —NR^{A}S(═O)₂SR^{A}. In certain embodiments, at least one occurrence of R² is —NR^{A}S(═O)₂N(R^{A})₂. In certain embodiments, at least one occurrence of R² is —Si(R^{A})₃. In certain embodiments, at least one occurrence of R² is —Si(R^{A})₂OR^{A}. In certain embodiments, at least one of occurrence of R² is —Si(R^{A})(OR^{A})₂. In certain embodiments, at least one occurrence of R² is —Si(OR^{A})₃. In certain embodiments, at least one occurrence of R² is —OSi(R^{A})₃. In certain embodiments, at least one occurrence of R² is —OSi(R^{A})₂OR^{A}. In certain embodiments, at least one occurrence of R² is —OSi(R^{A})(OR^{A})₂. In certain embodiments, at least one occurrence of R² is —OSi(OR^{A})₃. In certain embodiments, at least one occurrence of R² is —B(OR^{A})₂. In certain embodiments, two occurrences of R² are taken together to form ═O. In certain embodiments, two occurrences of R² are taken together to form ═S. In certain embodiments, two occurrences of R² are taken together to form an optionally substituted carbocyclyl. In certain embodiments, two occurrences of R² are taken together to form an optionally substituted heterocyclyl.

In certain embodiments, m1 is 0, 1, 2, 3, 4, 5, 6, 7, 8, or 9. In certain embodiments, m1 is 0. In certain embodiments, m1 is 1. In certain embodiments, m1 is 2. In certain embodiments, m1 is 3. In certain embodiments, m1 is 4. In certain embodiments, m1 is 5. In certain embodiments, m1 is 6. In certain embodiments, m1 is 7. In certain embodiments, m1 is 8. In certain embodiments, m1 is 9.

In certain embodiments, m2 is 0, 1, 2, 3, 4, 5, 6, or 7. In certain embodiments, m2 is 0. In certain embodiments, m2 is 1. In certain embodiments, m2 is 2. In certain embodiments, m2 is 3. In certain embodiments, m2 is 4. In certain embodiments, m2 is 5. In certain embodiments, m2 is 6. In certain embodiments, m2 is 7.

In certain embodiments, the compound of Formula (I-a) is:

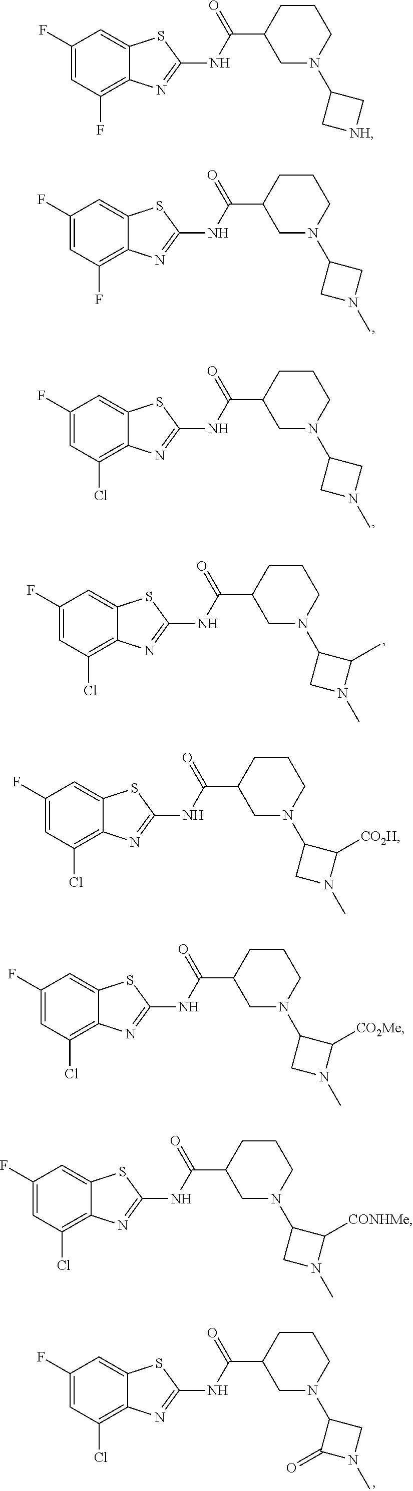

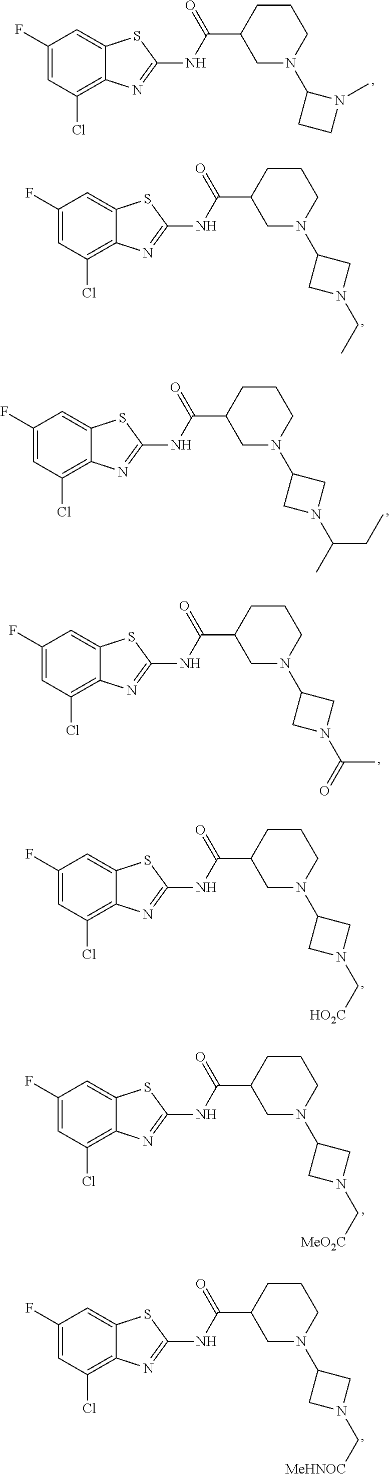

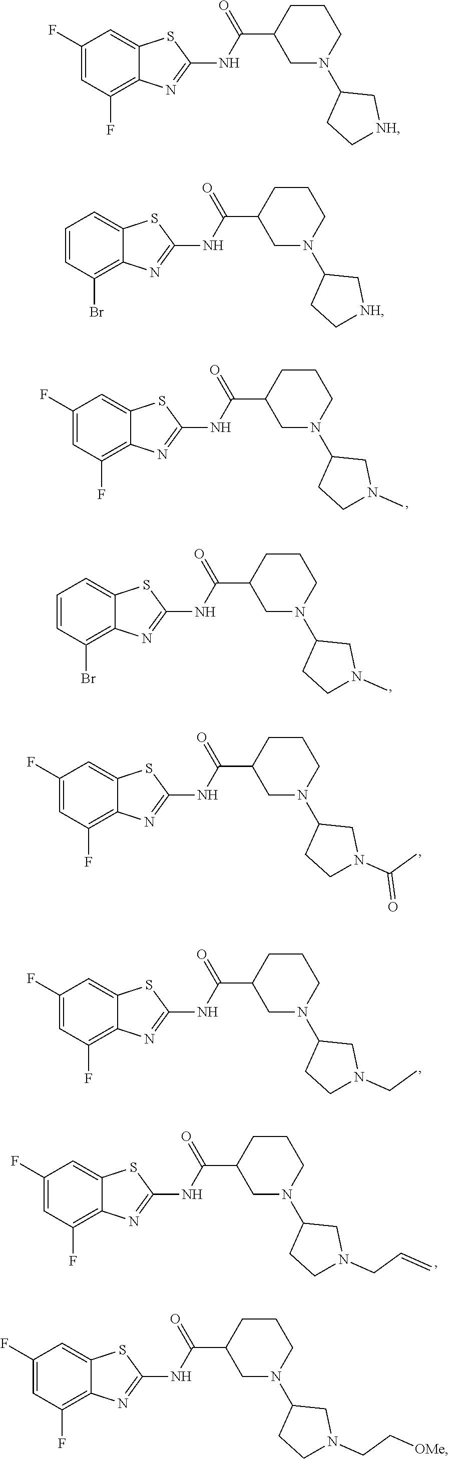

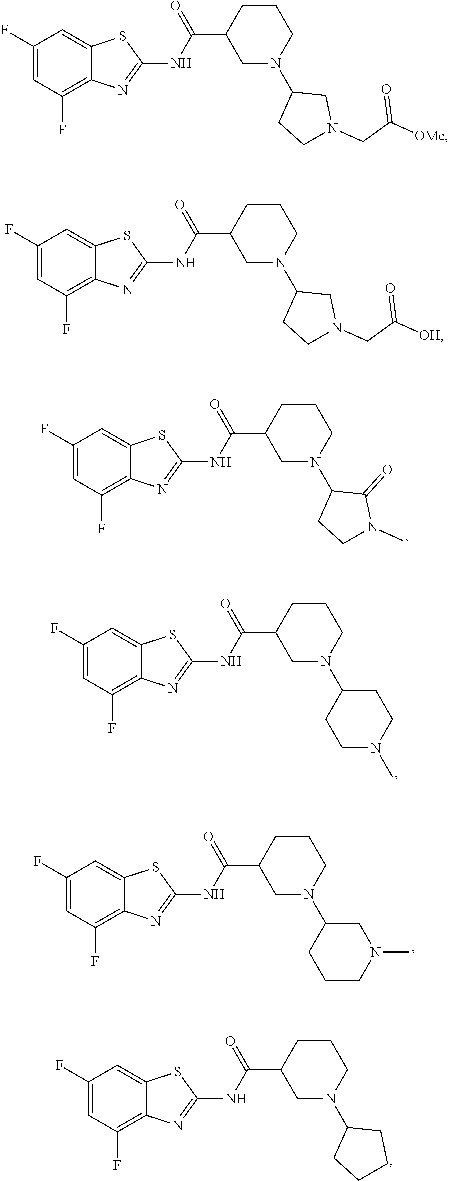 [SMILES: COC(=O)CN1CCC(N2CCCC(C(=O)Nc3nc4c(F)cc(F)cc4s3)C2)C1 ; O=C(O)CN1CCC(N2CCCC(C(=O)Nc3nc4c(F)cc(F)cc4s3)C2)C1 ; CN1CCC(N2CCCC(C(=O)Nc3nc4c(F)cc(F)cc4s3)C2)C1=O ; CN1CCC(N2CCCC(C(=O)Nc3nc4c(F)cc(F)cc4s3)C2)CC1 ; CN1CCCC(N2CCCC(C(=O)Nc3nc4c(F)cc(F)cc4s3)C2)C1 ; O=C(Nc1nc2c(F)cc(F)cc2s1)C1CCCN(C2CCCC2)C1]

or a pharmaceutically acceptable salt, solvate, hydrate, polymorph, co-crystal, tautomer, stereoisomer, isotopically labeled compound, or prodrug thereof.

In certain embodiments, the compound of Formula (I-a) is:

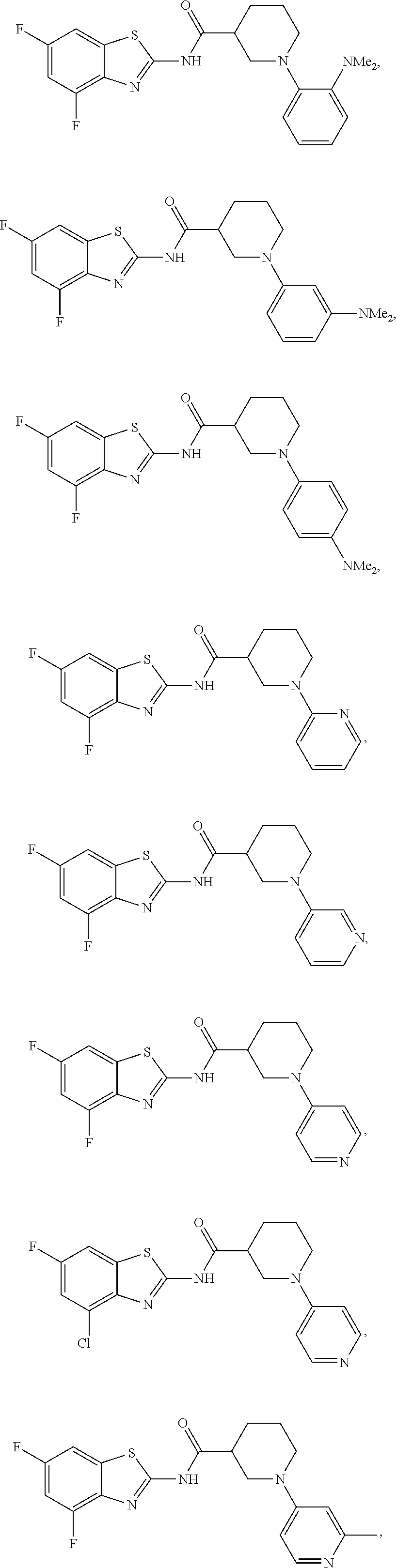

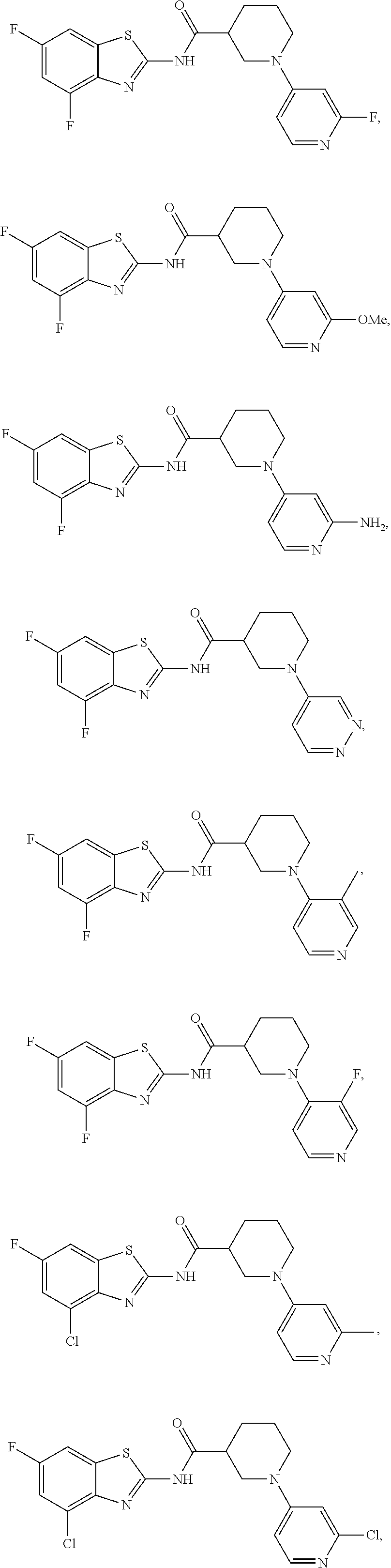

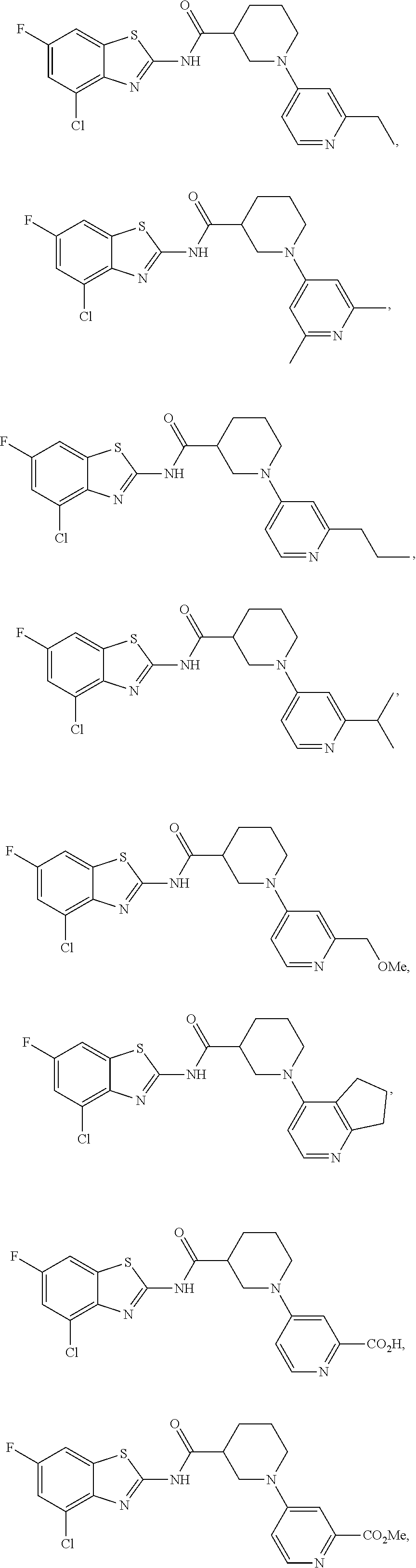

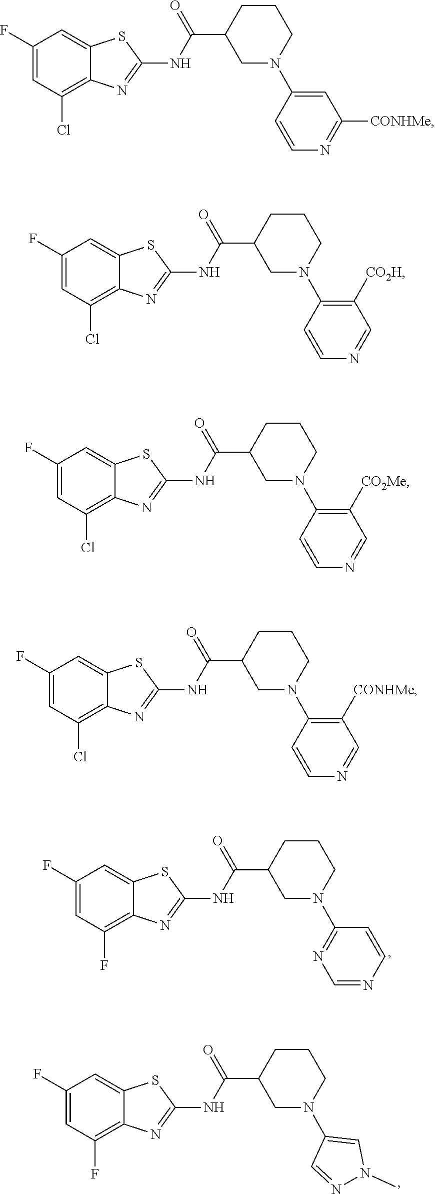

or a pharmaceutically acceptable salt, solvate, hydrate, polymorph, co-crystal, tautomer, stereoisomer, isotopically labeled compound, or prodrug thereof.

In certain embodiments, the compound of Formula (I-a) is:

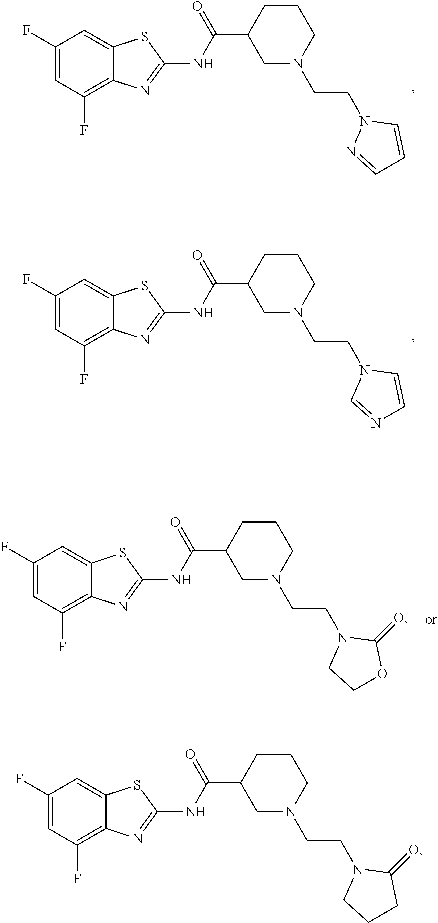 [SMILES: O=C(Nc1nc2c(F)cc(F)cc2s1)C1CCCN(CCn2cccn2)C1 ; O=C(Nc1nc2c(F)cc(F)cc2s1)C1CCCN(CCn2ccnc2)C1 ; O=C(Nc1nc2c(F)cc(F)cc2s1)C1CCCN(CCN2CCOC2=O)C1 ; O=C(Nc1nc2c(F)cc(F)cc2s1)C1CCCN(CCN2CCCC2=O)C1]

or a pharmaceutically acceptable salt, solvate, hydrate, polymorph, co-crystal, tautomer, stereoisomer, isotopically labeled compound, or prodrug thereof.

In certain embodiments, the compound of Formula (I-a) is not

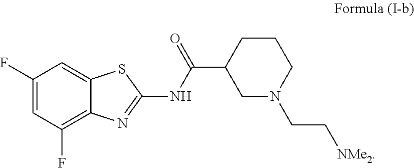 [SMILES: CN(C)CCN1CCCC(C(=O)Nc2nc3c(F)cc(F)cc3s2)C1]

### Ring B and R^{X}

In certain embodiments, Ring B is optionally substituted 5-6 membered heterocyclyl-R^{X}, wherein the optionally substituted 5-6 membered heterocyclyl comprises at least one ring oxo or thiooxo substituent. In certain embodiments, Ring B is optionally substituted 5-6 membered heterocyclyl-R^{X}, wherein the optionally substituted 5-6 membered heterocyclyl comprises at least one ring oxo substituent. In certain embodiments, Ring B is optionally substituted 5-6 membered heterocyclyl-R^{X}, wherein the optionally substituted 5-6 membered heterocyclyl comprises at least one ring thiooxo substituent. In certain embodiments, Ring B is optionally substituted pyrrolidinyl-R^{X} comprising at least one ring oxo or thiooxo substituent, optionally substituted piperidinyl-R^{X} comprising at least one ring oxo or thiooxo substituent, optionally substituted morpholinyl-R^{X} comprising at least one ring oxo or thiooxo substituent, optionally substituted tetrahydrofuranyl-R^{X} comprising at least one ring oxo or thiooxo substituent, optionally substituted tetrahydropyranyl-R^{X} comprising at least one ring oxo or thiooxo substituent. In certain embodiments, Ring B is optionally substituted pyrrolidinyl-R^{X} comprising at least one ring oxo substituent, optionally substituted piperidinyl-R^{X} comprising at least one ring oxo substituent, optionally substituted morpholinyl-R^{X} comprising at least one ring oxo substituent, optionally substituted tetrahydrofuranyl-R^{X} comprising at least one ring oxo substituent, optionally substituted tetrahydropyranyl-R^{X} comprising at least one ring oxo substituent. In certain embodiments, Ring B is optionally substituted pyrrolidinyl-R^{X} comprising at least one ring thiooxo substituent, optionally substituted piperidinyl-R^{X} comprising at least one ring thiooxo substituent, optionally substituted morpholinyl-R^{X} comprising at least one ring thiooxo substituent, optionally substituted tetrahydrofuranyl-R^{X} comprising at least one ring thiooxo substituent, optionally substituted tetrahydropyranyl-R^{X} comprising at least one ring thiooxo substituent. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, optionally substituted 2-oxomorpholinyl-R^{X}, optionally substituted 3-oxomorpholinyl-R^{X}, optionally substituted oxolan-2-onyl-R^{X}, optionally substituted oxolan-3-onyl-R^{X}, optionally substituted oxan-2-onyl-R^{X}, optionally substituted oxan-3-onyl-R^{X}, or optionally substituted oxan-4-onyl-R^{X}. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}.

In certain embodiments, Ring B is not optionally substituted

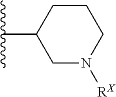

not comprising at least one ring oxo or thiooxo substituent,

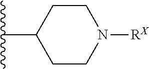

not comprising at least one ring oxo or thiooxo substituent, or

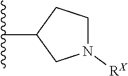

not comprising at least one ring oxo or thiooxo substituent.

In certain embodiments, R^{X} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A} —NR^{A}C(═NR^{A})OR^{A} —NR^{A}C(═NR^{A})SR^{A}, —NR^{A}C(═NR^{A})N(R^{A})₂, —NR^{A}S(═O)R^{A}, —NR^{A}S(═O)OR^{A} —NR^{A}S(═O)SR^{A}, —NR^{A}S(═O)N(R^{A})₂, —NR^{A}S(═O)₂R^{A}, —NR^{A}S(═O)₂OR^{A} —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, R^{X} is hydrogen. In certain embodiments, R^{X} is halogen. In certain embodiments, R^{X} is optionally substituted alkyl. In certain embodiments, R^{X} is optionally substituted alkenyl. In certain embodiments, R^{X} is optionally substituted alkynyl. In certain embodiments, R^{X} is optionally substituted heteroalkyl. In certain embodiments, R^{X} is optionally substituted heteroalkenyl. In certain embodiments, R^{X} is optionally substituted heteroalkynyl. In certain embodiments, R^{X} is optionally substituted carbocyclyl. In certain embodiments, R^{X} is optionally substituted heterocyclyl. In certain embodiments, R^{X} is optionally substituted aryl. In certain embodiments, R^{X} is optionally substituted heteroaryl. In certain embodiments, R^{X} is —CN. In certain embodiments, R^{X} is —OR^{A}. In certain embodiments, R^{X} is —SCN. In certain embodiments, R^{X} is —SR^{A}. In certain embodiments, R^{X} is —SSR^{A}. In certain embodiments, R^{X} is —N₃. In certain embodiments, R^{X} is —NO. In certain embodiments, R^{X} is —N(R^{A})₂. In certain embodiments, R^{X} is —NO₂. In certain embodiments, R^{X} is —C(═O)R^{A}. In certain embodiments, R^{X} is —C(═O)OR^{A}. In certain embodiments, R^{X} is —C(═O)SR^{A}. In certain embodiments, R^{X} is —C(═O)N(R^{A})₂. In certain embodiments, R^{X} is —C(═NR^{A})R^{A}. In certain embodiments, R^{X} is —C(═NR^{A})OR^{A}. In certain embodiments, R^{X} is —C(═NR^{A})SR^{A}. In certain embodiments, R^{X} is —C(═NR^{A})N(R^{A})₂. In certain embodiments, R^{X} is —S(═O)R^{A}. In certain embodiments, R^{X} is —S(═O)OR^{A}. In certain embodiments, R^{X} is —S(═O)SR^{A}. In certain embodiments, R^{X} is —S(═O)N(R^{A})₂. In certain embodiments, R^{X} is —S(═O)₂R^{A}. In certain embodiments, R^{X} is —S(═O)₂OR^{A}. In certain embodiments, R^{X} is —S(═O)₂SR^{A}. In certain embodiments, R^{X} is —S(═O)₂N(R^{A})₂. In certain embodiments, R^{X} is —OC(═O)R^{A}. In certain embodiments, R^{X} is —OC(═O)OR^{A}. In certain embodiments, R^{X} is —OC(═O)SR^{A}. In certain embodiments, R^{X} is —OC(═O)N(R^{A})₂. In certain embodiments, R^{X} is —OC(═NR^{A})R^{A}. In certain embodiments, R^{X} is —OC(═NR^{A})OR^{A}. In certain embodiments, R^{X} is —OC(═NR^{A})SR^{A}. In certain embodiments, R^{X} is —OC(═NR^{A})N(R^{A})₂. In certain embodiments, R^{X} is —OS(═O)R^{A}. In certain embodiments, R^{X} is —OS(═O)OR^{A}. In certain embodiments, R^{X} is —OS(═O)SR^{A}. In certain embodiments, R^{X} is —OS(═O)N(R^{A})₂. In certain embodiments, R^{X} is —OS(═O)₂R^{A}. In certain embodiments, R^{X} is —OS(═O)₂OR^{A}. In certain embodiments, R^{X} is —OS(═O)₂SR^{A}. In certain embodiments, R^{X} is —OS(═O)₂N(R^{A})₂. In certain embodiments, R^{X} is —ON(R^{A})₂. In certain embodiments, R^{X} is —SC(═O)R^{A}. In certain embodiments, R^{X} is —SC(═O)OR^{A}. In certain embodiments, R^{X} is —SC(═O)SR^{A}. In certain embodiments, R^{X} is —SC(═O)N(R^{A})₂. In certain embodiments, R^{X} is —SC(═NR^{A})R^{A}. In certain embodiments, R^{X} is —SC(═NR^{A})OR^{A}. In certain embodiments, R^{X} is —SC(═NR^{A})SR^{A}. In certain embodiments, R^{X} is —SC(═NR^{A})N(R^{A})₂. In certain embodiments, R^{X} is —NR^{A}C(═O)R^{A}. In certain embodiments, R^{X} is —NR^{A}C(═O)OR^{A}. In certain embodiments, R^{X} is —NR^{A}C(═O)SR^{A}. In certain embodiments, R^{X} is —NR^{A}C(═O)N(R^{A})₂. In certain embodiments, R^{X} is —NR^{A}C(═NR^{A})R^{A}. In certain embodiments, R^{X} is —NR^{A}C(═NR^{A})OR^{A}. In certain embodiments, R^{X} is —NR^{A}C(═NR^{A})SR^{A}. In certain embodiments, R^{X} is —NR^{A}C(═NR^{A})N(R^{A})₂. In certain embodiments, R^{X} is —NR^{A}S(═O)R^{A}In certain embodiments, R^{X} is —NR^{A}S(═O)OR^{A}. In certain embodiments, R^{X} is —NR^{A}S(═O)SR^{A}. In certain embodiments, R^{X} is —NR^{A}S(═O)N(R^{A})₂. In certain embodiments, R^{X} is —NR^{A}S(═O)₂R^{A}. In certain embodiments, R^{X} is —NR^{A}S(═O)₂OR^{A}. In certain embodiments, R^{X} is —NR^{A}S(═O)₂SR^{A}. In certain embodiments, R^{X} is —NR^{A}S(═O)₂N(R^{A})₂. In certain embodiments, R^{X} is —Si(R^{A})₃. In certain embodiments, R^{X} is —Si(R^{A})₂OR^{A}. In certain embodiments, at least one of occurrence of R^{X} is —Si(R^{A})(OR^{A})₂. In certain embodiments, R^{X} is —Si(OR^{A})₃. In certain embodiments, R^{X} is —OSi(R^{A})₃. In certain embodiments, R^{X} is —OSi(R^{A})₂OR^{A}. In certain embodiments, R^{X} is —OSi(R^{A})(OR^{A})₂. In certain embodiments, R^{X} is —OSi(OR^{A})₃. In certain embodiments, R^{X} is —B(OR^{A})₂.

In certain embodiments, R^{X} is optionally substituted alkyl. In certain embodiments, R^{X} is optionally substituted C₁₋₁₂ alkyl. In certain embodiments, R^{X} is optionally substituted C₁₋₆ alkyl. In certain embodiments, R^{X} is optionally substituted C₁₋₃ alkyl. In certain embodiments, R^{X} is optionally substituted methyl, optionally substituted ethyl, optionally substituted n-propyl, or optionally substituted isopropyl. In certain embodiments, R^{X} is optionally substituted alkyl substituted with —N(R^{A})₂. In certain embodiments, R^{X} is optionally substituted C₁₋₆ alkyl substituted with —N(R^{A})₂. In certain embodiments, R^{X} is optionally substituted C₁₋₃ alkyl substituted with —N(R^{A})₂. In certain embodiments, R^{X} is optionally substituted methyl substituted with —N(R^{A})₂, optionally substituted ethyl substituted with —N(R^{A})₂, optionally substituted n-propyl substituted with —N(R^{A})₂, or optionally substituted isopropyl substituted with —N(R^{A})₂. In certain embodiments, R^{X} is optionally substituted alkyl substituted with —NR^{A}(optionally substituted alkyl). In certain embodiments, R^{X} is optionally substituted C₁₋₆ alkyl substituted with —NR^{A}(optionally substituted alkyl). In certain embodiments, R^{X} is optionally substituted C₁₋₃ alkyl substituted with —NR^{A}(optionally substituted alkyl). In certain embodiments, R^{X} is optionally substituted methyl substituted with —NR^{A}(optionally substituted alkyl), optionally substituted ethyl substituted with —NR^{A}(optionally substituted alkyl), optionally substituted n-propyl substituted with —NR^{A}(optionally substituted alkyl), or optionally substituted isopropyl substituted with —NR^{A}(optionally substituted alkyl). In certain embodiments, R^{X} is optionally substituted alkyl substituted with —N(optionally substituted alkyl)₂. In certain embodiments, R^{X} is optionally substituted C₁₋₆ alkyl substituted with —N(optionally substituted alkyl)₂. In certain embodiments, R^{X} is optionally substituted C₁₋₃ alkyl substituted with —N(optionally substituted alkyl)₂. In certain embodiments, R^{X} is optionally substituted methyl substituted with —N(optionally substituted alkyl)₂, optionally substituted ethyl substituted with —N(optionally substituted alkyl)₂, optionally substituted n-propyl substituted with —N(optionally substituted alkyl)₂, or optionally substituted isopropyl substituted with —N(optionally substituted alkyl)₂. In certain embodiments, R^{X} is optionally substituted alkyl substituted with —NMe₂. In certain embodiments, R^{X} is optionally substituted C₁₋₆ alkyl substituted with —NMe₂. In certain embodiments, R^{X} is optionally substituted C₁₋₃ alkyl substituted with —NMe₂. In certain embodiments, R^{X} is optionally substituted methyl substituted with —NMe₂, optionally substituted ethyl substituted with —NMe₂, optionally substituted n-propyl substituted with —NMe₂, or optionally substituted isopropyl substituted with —NMe₂. In certain embodiments, R^{X} is

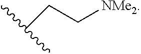 [SMILES: *CCN(C)C]

In certain embodiments, R^{X} is optionally substituted alkyl substituted with —NR^{A}(optionally substituted acyl). In certain embodiments, R^{X} is optionally substituted alkyl substituted with —NR^{A}(optionally substituted alkenyl). In certain embodiments, R^{X} is optionally substituted alkyl substituted with —NR^{A}(optionally substituted alkynyl). In certain embodiments, R^{X} is optionally substituted alkyl substituted with —NR^{A}(optionally substituted heteroalkyl). In certain embodiments, R^{X} is optionally substituted alkyl substituted with —NR^{A}(optionally substituted heteroalkenyl). In certain embodiments, R^{X} is optionally substituted alkyl substituted with —NR^{A}(optionally substituted heteroalkynyl). In certain embodiments, R^{X} is optionally substituted alkyl substituted with —NR^{A}(optionally substituted carbocyclyl). In certain embodiments, R^{X} is optionally substituted alkyl substituted with —NR^{A}(optionally substituted heterocyclyl). In certain embodiments, R^{X} is optionally substituted alkyl substituted with —NR^{A}(optionally substituted aryl). In certain embodiments, R^{X} is optionally substituted alkyl substituted with —NR^{A}(optionally substituted heteroaryl). In certain embodiments, R^{X} is optionally substituted alkyl substituted with —NR^{A}(nitrogen protecting group). In certain embodiments, R^{X} is optionally substituted alkyl substituted with —NHRA In certain embodiments, R^{X} is optionally substituted alkyl substituted with —NH(optionally substituted acyl). In certain embodiments, R^{X} is optionally substituted alkyl substituted with —NH(optionally substituted alkyl). In certain embodiments, R^{X} is optionally substituted alkyl substituted with —NH(optionally substituted alkenyl). In certain embodiments, R^{X} is optionally substituted alkyl substituted with —NH(optionally substituted alkynyl). In certain embodiments, R^{X} is optionally substituted alkyl substituted with —NH(optionally substituted heteroalkyl). In certain embodiments, R^{X} is optionally substituted alkyl substituted with —NH(optionally substituted heteroalkenyl). In certain embodiments, R^{X} is optionally substituted alkyl substituted with —NH(optionally substituted heteroalkynyl). In certain embodiments, R^{X} is optionally substituted alkyl substituted with —NH(optionally substituted carbocyclyl). In certain embodiments, R^{X} is optionally substituted alkyl substituted with —NH(optionally substituted heterocyclyl). In certain embodiments, R^{X} is optionally substituted alkyl substituted with —NH(optionally substituted aryl). In certain embodiments, R^{X} is optionally substituted alkyl substituted with —NH(optionally substituted heteroaryl). In certain embodiments, R^{X} is optionally substituted alkyl substituted with —NH(nitrogen protecting group). In certain embodiments, R^{X} is optionally substituted alkyl substituted with —NH₂. In certain embodiments, R^{X} is optionally substituted alkyl substituted with —NHMe.

In certain embodiments, R^{X} is optionally substituted alkyl substituted with optionally substituted heterocyclyl. In certain embodiments, R^{X} is optionally substituted C₁₋₆ alkyl substituted with optionally substituted heterocyclyl. In certain embodiments, R^{X} is optionally substituted C₁₋₃ alkyl substituted with optionally substituted heterocyclyl. In certain embodiments, R^{X} is optionally substituted methyl substituted with optionally substituted heterocyclyl, optionally substituted ethyl substituted with optionally substituted heterocyclyl, optionally substituted n-propyl substituted with optionally substituted heterocyclyl, or optionally substituted isopropyl substituted with optionally substituted heterocyclyl. In certain embodiments, R^{X} is optionally substituted alkyl substituted with optionally substituted 3-14 membered heterocyclyl. In certain embodiments, R^{X} is optionally substituted alkyl substituted with optionally substituted 3-10 membered heterocyclyl. In certain embodiments, R^{X} is optionally substituted alkyl substituted with optionally substituted 4-6 membered heterocyclyl. In certain embodiments, R^{X} is optionally substituted C₁₋₆ alkyl substituted with optionally substituted 4-6 membered heterocyclyl. In certain embodiments, R^{X} is optionally substituted C₁₋₃ alkyl substituted with optionally substituted 4-6 membered heterocyclyl. In certain embodiments, R^{X} is optionally substituted methyl substituted with optionally substituted 4-6 membered heterocyclyl, optionally substituted ethyl substituted with optionally substituted 4-6 membered heterocyclyl, optionally substituted n-propyl substituted with optionally substituted 4-6 membered heterocyclyl, or optionally substituted isopropyl substituted with optionally substituted 4-6 membered heterocyclyl. In certain embodiments, R^{X} is optionally substituted alkyl substituted with optionally substituted aziridinyl, optionally substituted alkyl substituted with optionally substituted azetidinyl, optionally substituted alkyl substituted with optionally substituted pyrrolidinyl, or optionally substituted alkyl substituted with optionally substituted piperidinyl. In certain embodiments, R^{X} is optionally substituted alkyl substituted with optionally substituted piperidinyl. In certain embodiments, R^{X} is optionally substituted C₁₋₆ alkyl substituted with optionally substituted piperidinyl. In certain embodiments, R^{X} is optionally substituted C₁₋₃ alkyl substituted with optionally substituted piperidinyl. In certain embodiments, R^{X} is optionally substituted methyl substituted with optionally substituted piperidinyl, optionally substituted ethyl substituted with optionally substituted piperidinyl, optionally substituted n-propyl substituted with optionally substituted piperidinyl, or optionally substituted isopropyl substituted with optionally substituted piperidinyl. In certain embodiments, R^{X} is optionally substituted alkyl substituted with optionally substituted heterocyclyl substituted with —N(R^{A})₂. In certain embodiments, R^{X} is optionally substituted C₁₋₆ alkyl substituted with optionally substituted heterocyclyl substituted with —N(R^{A})₂. In certain embodiments, R^{X} is optionally substituted C₁₋₃ alkyl substituted with optionally substituted heterocyclyl substituted with —N(R^{A})₂. In certain embodiments, R^{X} is optionally substituted methyl substituted with optionally substituted heterocyclyl substituted with —N(R^{A})₂, optionally substituted ethyl substituted with optionally substituted heterocyclyl substituted with —N(R^{A})₂, optionally substituted n-propyl substituted with optionally substituted heterocyclyl substituted with —N(R^{A})₂, or optionally substituted isopropyl substituted with optionally substituted heterocyclyl substituted with —N(R^{A})₂. In certain embodiments, R^{X} is optionally substituted alkyl substituted with optionally substituted heterocyclyl substituted with —NR^{A}(optionally substituted alkyl). In certain embodiments, R^{X} is optionally substituted C₁₋₆ alkyl substituted with optionally substituted heterocyclyl substituted with —NR^{A}(optionally substituted alkyl). In certain embodiments, R^{X} is optionally substituted C₁₋₃ alkyl substituted with optionally substituted heterocyclyl substituted with —NR^{A}(optionally substituted alkyl). In certain embodiments, R^{X} is optionally substituted methyl substituted with optionally substituted heterocyclyl substituted with —NR^{A}(optionally substituted alkyl), optionally substituted ethyl substituted with optionally substituted heterocyclyl substituted with —NR^{A}(optionally substituted alkyl), optionally substituted n-propyl substituted with optionally substituted heterocyclyl substituted with —NR^{A}(optionally substituted alkyl), or optionally substituted isopropyl substituted with optionally substituted heterocyclyl substituted with —NR^{A}(optionally substituted alkyl). In certain embodiments, R^{X} is optionally substituted alkyl substituted with optionally substituted heterocyclyl substituted with —NMe₂. In certain embodiments, R^{X} is optionally substituted C₁₋₆ alkyl substituted with optionally substituted heterocyclyl substituted with —NMe₂. In certain embodiments, R^{X} is optionally substituted C₁₋₃ alkyl substituted with optionally substituted heterocyclyl substituted with —NMe₂. In certain embodiments, R^{X} is optionally substituted methyl substituted with optionally substituted heterocyclyl substituted with —NMe₂, optionally substituted ethyl substituted with optionally substituted heterocyclyl substituted with —NMe₂, optionally substituted n-propyl substituted with optionally substituted heterocyclyl substituted with —NMe₂, or optionally substituted isopropyl substituted with optionally substituted heterocyclyl substituted with —NMe₂. In certain embodiments, R^{X} is optionally substituted alkyl substituted with optionally substituted piperidinyl substituted with —N(R^{A})₂. In certain embodiments, R^{X} is optionally substituted C₁₋₆ alkyl substituted with optionally substituted piperidinyl substituted with —N(R^{A})₂. In certain embodiments, R^{X} is optionally substituted C₁₋₃ alkyl substituted with optionally substituted piperidinyl substituted with —N(R^{A})₂. In certain embodiments, R^{X} is optionally substituted methyl substituted with optionally substituted piperidinyl substituted with —N(R^{A})₂, optionally substituted ethyl substituted with optionally substituted piperidinyl substituted with —N(R^{A})₂, optionally substituted n-propyl substituted with optionally substituted piperidinyl substituted with —N(R^{A})₂, or optionally substituted isopropyl substituted with optionally substituted piperidinyl substituted with —N(R^{A})₂. In certain embodiments, R^{X} is optionally substituted alkyl substituted with optionally substituted piperidinyl substituted with —NR^{A}(optionally substituted alkyl). In certain embodiments, R^{X} is optionally substituted C₁₋₆ alkyl substituted with optionally substituted piperidinyl substituted with —NR^{A}(optionally substituted alkyl). In certain embodiments, R^{X} is optionally substituted C₁₋₃ alkyl substituted with optionally substituted piperidinyl substituted with —NR^{A}(optionally substituted alkyl). In certain embodiments, R^{X} is optionally substituted methyl substituted with optionally substituted piperidinyl substituted with —NR^{A}(optionally substituted alkyl), optionally substituted ethyl substituted with optionally substituted piperidinyl substituted with —NR^{A}(optionally substituted alkyl), optionally substituted n-propyl substituted with optionally substituted piperidinyl substituted with —NR^{A}(optionally substituted alkyl), or optionally substituted isopropyl substituted with optionally substituted piperidinyl substituted with —NR^{A}(optionally substituted alkyl). In certain embodiments, R^{X} is optionally substituted alkyl substituted with optionally substituted piperidinyl substituted with —NMe₂. In certain embodiments, R^{X} is optionally substituted C₁₋₆ alkyl substituted with optionally substituted piperidinyl substituted with —NMe₂. In certain embodiments, R^{X} is optionally substituted C₁₋₃ alkyl substituted with optionally substituted piperidinyl substituted with —NMe₂. In certain embodiments, R^{X} is optionally substituted methyl substituted with optionally substituted piperidinyl substituted with —NMe₂, optionally substituted ethyl substituted with optionally substituted piperidinyl substituted with —NMe₂, optionally substituted n-propyl substituted with optionally substituted piperidinyl substituted with —NMe₂, or optionally substituted isopropyl substituted with optionally substituted piperidinyl substituted with —NMe₂.

In certain embodiments, Ring A is of formula

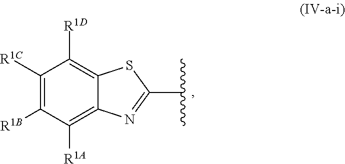

and Ring B is optionally substituted 5-6 membered heterocyclyl-R^{X}, wherein the optionally substituted 5-6 membered heterocyclyl comprises at least one ring oxo or thiooxo substituent. In certain embodiments, Ring A is of formula

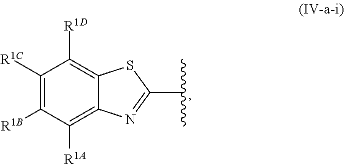

and Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}. In certain embodiments, Ring A is of formula

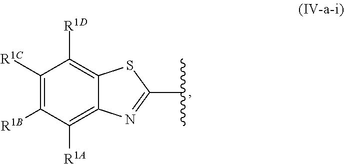

L is of formula

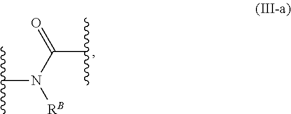

and Ring B is optionally substituted 5-6 membered heterocyclyl-R^{X}, wherein the optionally substituted 5-6 membered heterocyclyl comprises at least one ring oxo or thiooxo substituent. In certain embodiments, Ring A is of formula

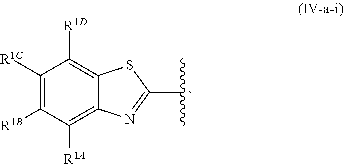

L is of formula

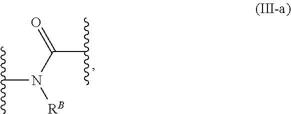

and Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}.

In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted alkyl. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted C₁₋₆ alkyl. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted C₁₋₃ alkyl. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted methyl, optionally substituted ethyl, optionally substituted n-propyl, or optionally substituted isopropyl. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted alkyl substituted with —N(R^{A})₂. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted methyl substituted with —N(R^{A})₂, optionally substituted ethyl substituted with —N(R^{A})₂, optionally substituted n-propyl substituted with —N(R^{A})₂, or optionally substituted isopropyl substituted with —N(R^{A})₂. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted alkyl substituted with —NR^{A}(optionally substituted alkyl. In certain embodiments, Ring B is optionally substituted 5-6 membered heterocyclyl-R^{X1}, wherein the optionally substituted 5-6 membered heterocyclyl comprises at least one ring oxo or thiooxo substituent, and R^{X} is optionally substituted methyl substituted with —NR^{A}(optionally substituted alkyl), optionally substituted ethyl substituted with —NR^{A}(optionally substituted alkyl), optionally substituted n-propyl substituted with —NR^{A}(optionally substituted alkyl), or optionally substituted isopropyl substituted with —NR^{A}(optionally substituted alkyl). In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted alkyl substituted with —N(optionally substituted alkyl)₂. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted methyl substituted with —N(optionally substituted alkyl)₂, optionally substituted ethyl substituted with —N(optionally substituted alkyl)₂, optionally substituted n-propyl substituted with —N(optionally substituted alkyl)₂, or optionally substituted isopropyl substituted with —N(optionally substituted alkyl)₂. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted alkyl substituted with —NMe₂. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is optionally substituted methyl substituted with —NMe₂, optionally substituted ethyl substituted with —NMe₂, optionally substituted n-propyl substituted with —NMe₂, or optionally substituted isopropyl substituted with —NMe₂. In certain embodiments, Ring B is optionally substituted 5-6 membered heterocyclyl-R^{X}, wherein the optionally substituted 5-6 membered heterocyclyl comprises at least one ring oxo or thiooxo substituent, and R^{X} is

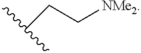 [SMILES: *CCN(C)C]

In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted alkyl. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted C₁₋₆ alkyl. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted C₁₋₃ alkyl. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted methyl, optionally substituted ethyl, optionally substituted n-propyl, or optionally substituted isopropyl. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted alkyl substituted with —N(R^{A})₂. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted methyl substituted with —N(R^{A})₂, optionally substituted ethyl substituted with —N(R^{A})₂, optionally substituted n-propyl substituted with —N(R^{A})₂, or optionally substituted isopropyl substituted with —N(R^{A})₂. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted alkyl substituted with —NR^{A}(optionally substituted alkyl. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted methyl substituted with —NR^{A}(optionally substituted alkyl), optionally substituted ethyl substituted with —NR^{A}(optionally substituted alkyl), optionally substituted n-propyl substituted with —NR^{A}(optionally substituted alkyl), or optionally substituted isopropyl substituted with —NR^{A}(optionally substituted alkyl). In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted alkyl substituted with —N(optionally substituted alkyl)₂. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted methyl substituted with —N(optionally substituted alkyl)₂, optionally substituted ethyl substituted with —N(optionally substituted alkyl)₂, optionally substituted n-propyl substituted with —N(optionally substituted alkyl)₂, or optionally substituted isopropyl substituted with —N(optionally substituted alkyl)₂. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted alkyl substituted with —NMe₂. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is optionally substituted methyl substituted with —NMe₂, optionally substituted ethyl substituted with —NMe₂, optionally substituted n-propyl substituted with —NMe₂, or optionally substituted isopropyl substituted with —NMe₂. In certain embodiments, Ring B is optionally substituted 2-pyrrolidinonyl-R^{X}, optionally substituted 3-pyrrolidinonyl-R^{X}, optionally substituted 2-piperidinonyl-R^{X}, optionally substituted 3-piperidinonyl-R^{X}, optionally substituted 4-piperidinonyl-R^{X}, or optionally substituted 2-oxomorpholinyl-R^{X}, and R^{X} is

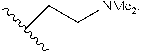 [SMILES: *CCN(C)C]

In certain embodiments, the compound of Formula (I-b) is:

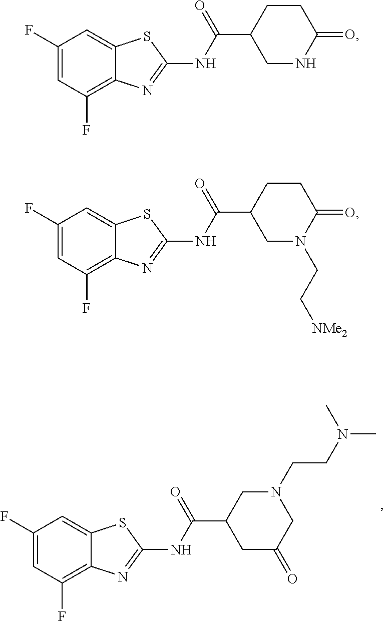 [SMILES: O=C1CCC(C(=O)Nc2nc3c(F)cc(F)cc3s2)CN1 ; CN(C)CCN1CC(C(=O)Nc2nc3c(F)cc(F)cc3s2)CCC1=O ; CN(C)CCN1CC(=O)CC(C(=O)Nc2nc3c(F)cc(F)cc3s2)C1]

or a pharmaceutically acceptable salt, solvate, hydrate, polymorph, co-crystal, tautomer, stereoisomer, isotopically labeled compound, or prodrug thereof.

In certain embodiments, the compound of Formula (I-b) is not

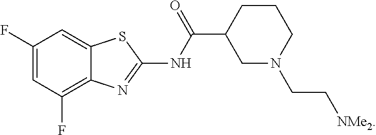 [SMILES: CN(C)CCN1CCCC(C(=O)Nc2nc3c(F)cc(F)cc3s2)C1]

In certain embodiments, disclosed is a compound of formula:

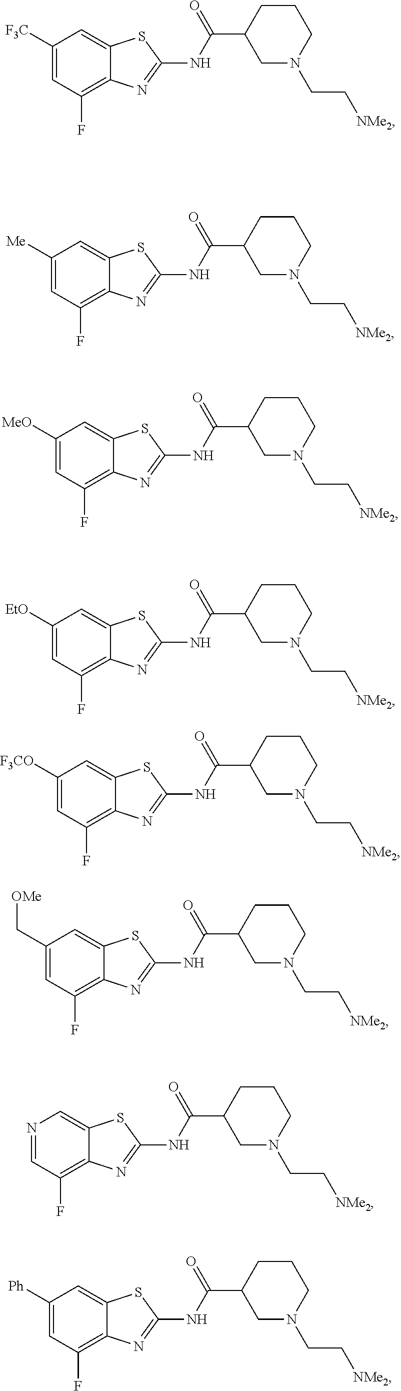

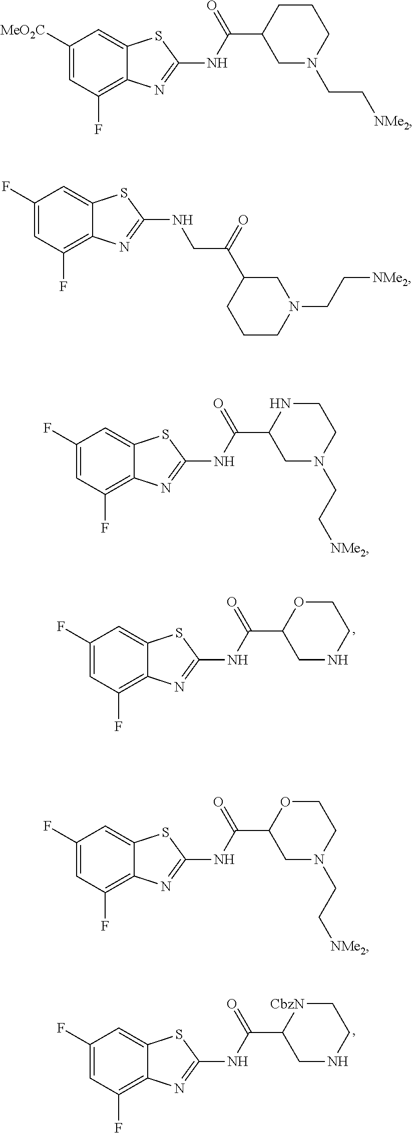 [SMILES: COC(=O)c1cc(F)c2nc(NC(=O)C3CCCN(CCN(C)C)C3)sc2c1 ; CN(C)CCN1CCCC(C(=O)CNc2nc3c(F)cc(F)cc3s2)C1 ; CN(C)CCN1CCNC(C(=O)Nc2nc3c(F)cc(F)cc3s2)C1 ; O=C(Nc1nc2c(F)cc(F)cc2s1)C1CNCCO1 ; CN(C)CCN1CCOC(C(=O)Nc2nc3c(F)cc(F)cc3s2)C1 ; O=C(Nc1nc2c(F)cc(F)cc2s1)C1CNCCN1C(=O)OCc1ccccc1]

or a pharmaceutically acceptable salt, solvate, hydrate, polymorph, co-crystal, tautomer, stereoisomer, isotopically labeled compound, or prodrug thereof.

In certain embodiments, a provided compound (a compound described herein, a compound of the present disclosure) is a compound of any of the formulae herein (e.g., Formulae (I-a) or (I-b)), or pharmaceutically acceptable salt, solvate, hydrate, polymorph, co-crystal, tautomer, stereoisomer, isotopically labeled compound, or prodrug thereof. In certain embodiments, a provided compound is a compound of any of the formulae herein (e.g., Formulae (I-a) or (I-b)), or a pharmaceutically acceptable salt or tautomer thereof. In certain embodiments, a provided compound is a compound of any of the formulae herein (e.g., Formulae (I-a) or (I-b)), or a pharmaceutically acceptable salt thereof. In certain embodiments, a provided compound is a compound of Formula (I-a) or Formula (I-b), or a salt thereof.

In certain embodiments, the provided compound is not

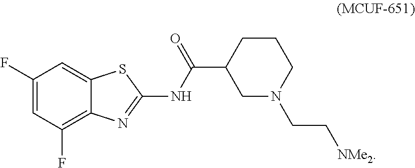 [SMILES: CN(C)CCN1CCCC(C(=O)Nc2nc3c(F)cc(F)cc3s2)C1]

### Pharmaceutical Compositions and Kits

In one aspect, the present disclosure provides pharmaceutical compositions comprising a provided compound. In some embodiments, the pharmaceutical composition comprises one or more excipients. In certain embodiments, the pharmaceutical compositions described herein comprise a provided compound and an excipient.

In certain embodiments, the pharmaceutical composition comprises an effective amount of the provided compound. In certain embodiments, the effective amount is a therapeutically effective amount. In certain embodiments, the effective amount is a prophylactically effective amount. In certain embodiments, the effective amount is an amount effective for treating a disease or disorder associated with associated with particulate guanylyl cyclase-A (pGC-A) (e.g., cardiovascular disease, metabolic disease, kidney disease) in a subject in need thereof. In certain embodiments, the effective amount is an amount effective for preventing a disease or disorder associated with pGC-A (e.g., cardiovascular disease, metabolic disease, kidney disease) in a subject in need thereof. In certain embodiments, the effective amount is an amount effective for reducing the risk of developing a disease or disorder associated with pGC-A (e.g., cardiovascular disease, metabolic disease, kidney disease) in a subject in need thereof. In certain embodiments, the effective amount is an amount effective for modulating pGC-A in a subject in need thereof or in a cell, tissue, or biological sample. In certain embodiments, the effective amount is an amount effective for enhancing an activity of pGC-A. In certain embodiments, the effective amount is an amount effective for positive allosteric enhancement of activity of pGC-A. In certain embodiments, the effective amount is an amount effective for producing cyclic 3′,5′-guanosine monophosphate (cGMP). In certain embodiments, the effective amount is an amount effective for increasing production of cGMP.

In certain embodiments, the subject is an animal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years, inclusive. In certain embodiments, the subject is a human younger than 2 years. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for enhancing activity of pGC-A by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%. In certain embodiments, the effective amount is an amount effective for enhancing activity of pGC-A by a range between a percentage described in this paragraph and another percentage described in this paragraph, inclusive.

In certain embodiments, the effective amount is an amount effective for increasing production of cGMP by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%. In certain embodiments, the effective amount is an amount effective for increasing production of cGMP by a range between a percentage described in this paragraph and another percentage described in this paragraph, inclusive.

In certain embodiments, the pharmaceutical composition is for use in treating a disease or disorder associated with pGC-A (e.g., cardiovascular disease, metabolic disease, kidney disease) in a subject in need thereof. In certain embodiments, the pharmaceutical composition is for use in preventing a disease or disorder associated with pGC-A (e.g., cardiovascular disease, metabolic disease, kidney disease) in a subject in need thereof. In certain embodiments, the pharmaceutical composition is for use in modulating pGC-A in a subject in need thereof or in a cell, tissue, or biological sample. In certain embodiments, the pharmaceutical composition is for use in enhancing pGC-A activity in a subject in need thereof or in a cell, tissue, or biological sample. In certain embodiments, the pharmaceutical composition is for use in positive allosteric enhancement of activity of pGC-A. In certain embodiments, the pharmaceutical composition is for use in producing cyclic 3′,5′-guanosine monophosphate (cGMP).

A provided compound or pharmaceutical composition, as described herein, can be administered in combination with one or more additional pharmaceutical agents (e.g., therapeutically and/or prophylactically active agents). The provided compounds or pharmaceutical compositions can be administered in combination with additional pharmaceutical agents that improve their activity (e.g., activity (e.g., potency and/or efficacy) in treating a disease or disorder associated with pGC-A in a subject in need thereof, in preventing a disease or disorder associated with pGC-A in a subject in need thereof, and/or in reducing the risk of developing a disease or disorder associated with pGC-A in a subject in need thereof), improve bioavailability, improve safety, reduce drug resistance, reduce and/or modify metabolism, inhibit excretion, and/or modify distribution in a subject or cell. It will also be appreciated that the additional pharmaceutical agents employed may achieve a desired effect for the same disorder, and/or it may achieve different effects. In certain embodiments, a pharmaceutical composition described herein including a provided compound described herein and an additional pharmaceutical agent exhibit a synergistic effect that is absent in a pharmaceutical composition including one of the provided compounds and the additional pharmaceutical agent, but not both. In some embodiments, the additional pharmaceutical agent achieves a desired effect for the same disorder. In some embodiments, the additional pharmaceutical agent achieves different effects.

The provided compound or pharmaceutical composition can be administered concurrently with, prior to, or subsequent to one or more additional pharmaceutical agents, which are different from the compound or pharmaceutical composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides, synthetic proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or disorder associated with pGC-A.

Each additional pharmaceutical agent may be administered at a dose and/or on a time schedule determined for that pharmaceutical agent. The additional pharmaceutical agents may also be administered together with each other and/or with the compound or pharmaceutical composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agents include, but are not limited to, cardiovascular agents, cholesterol-lowering agents, anti-diabetic agents, anti-coagulants, steroidal agents, hormones, and prostaglandins. In certain embodiments, the additional pharmaceutical agent is a cardiovascular agent. In certain embodiments, the additional pharmaceutical agent is a cholesterol-lowering agent. In certain embodiments, the additional pharmaceutical agent is an anti-diabetic agent. In certain embodiments, the additional pharmaceutical agent is an anti-coagulant. In certain embodiments, the additional pharmaceutical agent is a steroidal agent. In certain embodiments, the additional pharmaceutical agent is a hormone. In certain embodiments, the additional pharmaceutical agent is a prostaglandin.

In certain embodiments, the provided compound or pharmaceutical composition is a solid. In certain embodiments, the provided compound or pharmaceutical composition is a powder. In certain embodiments, the provided compound or pharmaceutical composition can be dissolved in a liquid to make a solution. In certain embodiments, the provided compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

Pharmaceutical compositions described herein can be prepared by any method known in the art of pharmacology. In general, such preparatory methods include the steps of bringing the composition comprising a provided compound (i.e., the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the provided compound, pharmaceutically acceptable excipient, agent, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) agent, inclusive.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid pharmaceutical compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Pharmaceutical compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Suitable devices for use in delivering injectable pharmaceutical compositions described herein include short needle devices. Injectable pharmaceutical compositions can be administered by devices which limit the effective penetration length of a needle into the skin. Alternatively or additionally, conventional syringes can be used in the classical mantoux method of administration. Jet injection devices which deliver liquid formulations via a liquid jet injector and/or via a needle. Ballistic powder/particle delivery devices which use compressed gas to accelerate the compound in powder form are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such pharmaceutical compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder pharmaceutical compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such pharmaceutical compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Provided compounds are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The provided compounds and pharmaceutical compositions provided herein can be administered by any route, including enteral (e.g., oral), parenteral, intravenous, intramuscular, intraarticular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are intraarticular administration, oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a provided compound required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, a pharmaceutical composition comprising a provided compound is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the pharmaceutical composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Dose ranges as described herein provide guidance for the administration of provided compounds or pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Also encompassed by the present disclosure are kits (e.g., pharmaceutical packs). In certain embodiments, the kit comprises a provided compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the provided compound or pharmaceutical composition included in the first container and the excipient or additional pharmaceutical agent included in the second container are combined to form one unit dosage form. In some embodiments, the provided compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the provided compound or pharmaceutical composition to a subject (e.g., a subject in need of treatment or prevention of a disease described herein). In certain embodiments, the instructions are for contacting a biological sample or cell with the provided compound or pharmaceutical composition. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

In certain embodiments, the kits and instructions provide for treating a disease or disorder associated with pGC-A in a subject in need thereof. In certain embodiments, the kits and instructions provide for preventing a disease or disorder associated with pGC-A in a subject in need thereof. In certain embodiments, the kits and instructions provide for reducing the risk of developing a disease or disorder associated with pGC-A in a subject in need thereof. In certain embodiments, the kits and instructions provide for modulating pGC-A in a subject in need thereof or in a cell, tissue, or biological sample. In certain embodiments, the kits and instructions provide for enhancing an activity of pGC-A. In certain embodiments, the kits and instructions provide for positive allosteric enhancement of activity of pGC-A. In certain embodiments, the kits and instructions provide for producing cyclic 3′,5′-guanosine monophosphate (cGMP). In certain embodiments, the kits and instructions provide for increasing production of cGMP.

A kit described herein may include one or more additional pharmaceutical agents described herein as a separate pharmaceutical composition.

Another object of the present disclosure is the use of a compound as described herein in the manufacture of a medicament for use in the treatment of a disorder or disease described herein. Another object of the present disclosure is the use of a compound as described herein for use in the treatment of a disorder or disease described herein.

### Methods of Treatment and Prevention

In another aspect, the present disclosure provides methods of treating or preventing a disease in a subject in need thereof, comprising administering to the subject in need thereof a provided compound or pharmaceutical composition. In certain embodiments, the present disclosure provides methods of treating a disease in a subject in need thereof, comprising administering to the subject in need thereof a provided compound or pharmaceutical composition. In certain embodiments, the present disclosure provides methods of preventing a disease in a subject in need thereof, comprising administering to the subject in need thereof a provided compound or pharmaceutical composition.

In another aspect, the present disclosure provides a provided compound or pharmaceutical composition for use in treating or preventing a disease in a subject in need thereof. In another aspect, the present disclosure provides a provided compound or pharmaceutical composition for use in treating a disease in a subject in need thereof. In another aspect, the present disclosure provides a provided compound or pharmaceutical composition for use in preventing a disease in a subject in need thereof.

In another aspect, the present disclosure provides a provided compound or pharmaceutical composition for use in the manufacture of a medicament for treatment or prevention of a disease in a subject in need thereof. In another aspect, the present disclosure provides a provided compound or pharmaceutical composition for use in the manufacture of a medicament for treatment of a disease in a subject in need thereof. In another aspect, the present disclosure provides a provided compound or pharmaceutical composition for use in the manufacture of a medicament for prevention of a disease in a subject in need thereof.

In certain embodiments, the disease is associated with pGC-A (e.g., cardiovascular disease, metabolic disease, kidney disease). In certain embodiments, the disease is a cardiovascular disease (e.g., heart failure, cardiomyopathy, hypertension, high blood pressure, myocardial infarction, arrhythmia). In certain embodiments, the cardiovascular disease is heart failure. In certain embodiments, the cardiovascular disease is cardiomyopathy. In certain embodiments, the cardiovascular disease is hypertension. In certain embodiments, the cardiovascular disease is high blood pressure. In certain embodiments, the cardiovascular disease is myocardial infarction. In certain embodiments, the cardiovascular disease is arrhythmia. In certain embodiments, the disease is a metabolic disease (e.g., obesity, hypertriglyceridemia, metabolic syndrome, insulin resistance, hyperinsulinemia, diabetes, acidemia). In certain embodiments, the metabolic disease is obesity. In certain embodiments, the metabolic disease is hypertriglyceridemia. In certain embodiments, the metabolic disease is metabolic syndrome. In certain embodiments, the metabolic disease is insulin resistance. In certain embodiments, the metabolic disease is hyperinsulinemia. In certain embodiments, the metabolic disease is diabetes. In certain embodiments, the metabolic disease is acidemia. In certain embodiments, the disease is a kidney disease (e.g., nephropathy, acute renal failure, chronic kidney disease, diabetic kidney disease, cardiorenal syndrome). In certain embodiments, the kidney disease is nephropathy. In certain embodiments, the kidney disease is acute renal failure. In certain embodiments, the kidney disease is chronic kidney disease. In certain embodiments, the kidney disease is diabetic kidney disease. In certain embodiments, the kidney disease is cardiorenal syndrome.

Methods of Modulating Particulate Guanylyl Cyclase-A (pGC-A)

In another aspect, the present disclosure provides methods of modulating particulate guanylyl cyclase-A (pGC-A) in a subject in need thereof or in a cell, tissue, or biological sample, comprising administering to the subject in need thereof or contacting the cell, tissue, or biological sample with an effective amount of a provided compound or pharmaceutical composition. In certain embodiments, the present disclosure provides methods of modulating pGC-A in a subject in need thereof, comprising administering to the subject in need thereof an effective amount of a provided compound or pharmaceutical composition. In certain embodiments, the present disclosure provides methods of modulating pGC-A in a subject in need thereof, comprising contacting the cell, tissue, or biological sample with an effective amount of a provided compound or pharmaceutical composition.

In certain embodiments, the cell, tissue, or biological sample is in vivo. In certain embodiments, the cell, tissue, or biological sample is in vitro. In certain embodiments, the method comprises enhancing an activity of pGC-A. In certain embodiments, the method comprises positive allosteric enhancement of activity of pGC-A. In certain embodiments, the method further comprises producing cyclic 3′,5′-guanosine monophosphate (cGMP). In certain embodiments, the method further comprises increasing production of cGMP.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Determination of cGMP Potentiation

Test compounds were evaluated for potentiation of cGMP in human GC-A overexpressing HEK293 cells (Cardiorenal Research Laboratory, Mayo Clinic). Test compounds at 10 mM DMSO stock concentration were dispensed into 384 well small volume white plates using Tecan D300e digital dispenser starting at 5 μM concentration and diluted 2-fold for 10-point concentration response. Wells were backfilled with DMSO such that the final concentration of DMSO in all wells was maintained at 0.3% DMSO. Atrial natriuretic peptide (ANP, Phoenix Pharmaceuticals) peptide ANP was prepared as working stock aliquots at 5 μM in phosphate-buffered solution (PBS) with 0.1% bovine serum albumin (BSA) and diluted to 0.1 mM in buffer+Tween20 at 0.3% and added to plates to a final concentration of 20 pM (EC₂₀). GC-A cells in suspension (10 ml) in OptiMem media (11058-021) supplemented with 2% fetal bovine serum were plated at 1×10⁶ cells/mL and assay plates were spun at 1000 rpm for 30 sec and incubated at RT for 30 min. GC-A-mediated activation of cGMP production was measured using cGMP CisBio detection homogenous time-resolved florescence (HTRF) competition assay using labeled-cGMP in human GC-A-overexpressing HEK293 cells. 5 μL of d-2 acceptor was dispensed into each well followed by addition of 5 μL of Anti-cGMP Eu-Cryptate antibody donor and plates were spun at 1000 rpm for 30 sec and incubated in the dark at RT for 1 hr. HTRF florescence was read on the ClarioStar microplate reader (BMG). A 100% response was determined from wells in the absence of compound and presence of saturating concentration of ANP (10 nM), and 0% response was determined from wells containing an EC₂₀ concentration of ANP determined for each new lot of ANP. EC₂₀ values of ANP were maintained in the range of 15-30 pM across all assay plates tested.

MCUF-651 was used as a control compound on each assay plate and did not deviate from the EC₅₀ range of 0.1-0.7 nM and Emax range of 75-85. Test compounds were tested in the absence of ANP to determine agonist activity. None of the compounds exhibited activity in the absence of ANP.

For selectivity assays, compounds were tested in human GC-B overexpressing HEK293 cells in the presence of an EC₂₀ concentration of CNP (3 nM), the endogenous ligand for GC-B. A 100% response was determined from wells in the absence of compound and presence of saturating concentration of CNP (50 nM), and 0% response was determined from wells containing an EC₂₀ concentration of CNP. MCUF-42 was used as a control compound on each assay plate for GCB assay. None of the compounds exhibited activity in GC-B assays.

Lipophilic ligand efficiency (LLE)=EC₅₀−cLogP, and is an estimate of the specificity of a molecule in binding to the target relative to partitioning into 1-octanol. A molecule with an LLE=zero based on Log P, where target affinity is equal to Log P, can be thought of as having the same affinity for its target as it does for 1-octanol, whereas a drug candidate with an LLE=6 has a one-million-fold higher affinity for its target compared to 1-octanol.

As shown in Tables 1-3 below, compounds of any of the formulae herein (e.g., Formulae (I-a) or (I-b)), or pharmaceutically acceptable salts, solvates, hydrates, polymorphs, co-crystals, tautomers, stereoisomers, isotopically labeled compounds, or prodrugs thereof, modulate particulate guanylyl cyclase-A (pGC-A).

Certain compounds of any of the formulae herein (e.g., Formulae (I-a) or (I-b)), or pharmaceutically acceptable salts, solvates, hydrates, polymorphs, co-crystals, tautomers, stereoisomers, isotopically labeled compounds, or prodrugs thereof, have increased potency in the activation of GC-A receptor by >10-fold, increased solubility, decreased volume of distribution, minimal off-target activity and resulting toxicity, and/or improved target selectivity (e.g., vs. particulate guanylyl cyclase-B (pGC-B), human ether-α-go-go related gene (hERG)) compared to MCUF-651, which is orally bioavailable and active in vivo.

Overall potency was improved by 10-fold with significant improvement in fold-selectivity (approximately 2 orders of magnitude) over hERG function. For example, compound JUF-S4-029 possesses high potency in activation of GC-A and reduced toxicity resulting from binding at hERG channel and other off-target toxicity.

A basic amine is important for GC-A activity, as indicated by loss of activity in the neutral amide in JUF-S4-014. Heteroaromatic rings are tolerated, as in JUF-S4-012. Tying up the two-carbon linker into aromatic phenyl or pyridine ring led to active analogs (e.g., JUF-S4-009, JUF-S4-026, Table 2). The 4-pyridyl derivative (JUF-S4-026) was more active than MCUF-651 but did not result in selectivity over hERG binding.

N-cycloalkyl derivatives were screened at the core piperidine group present in MCUF-651, which incorporated the two-carbon linker and the dimethylamino pendant group (Table 1). The N-cycloalkyl derivatives are structurally more rigid, and reducing rotational degrees of freedom can increase binding to GC-A and allosteric activation activity. All analogs in this group were significantly more potent than MCUF-651, and there was a direct correlation to the size of the ring; the analog with smallest 4-membered N-methyl azetidine resulted in ~10-fold more potent analog JUF-S4-029 vs. MCUF-651. JUF-S4-029 did not eliminate hERG binding, but because of its higher potency at GC-A, is 50-100-fold more selective for GC-A vs hERG than MCUF-651 is.

TABLE 1
Modulation of pGC-A for compounds of Formula (I-a).

| | | EC₅₀ | Eₘₐₓ | |
|---|---|---|---|---|
| Compound Structure | ID | (μM) | (%) | LLE |
| 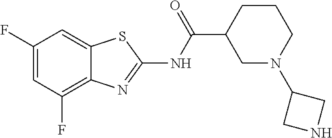 [SMILES: O=C(Nc1nc2c(F)cc(F)cc2s1)C1CCCN(C2CNC2)C1] | JUF-S4- 029A | 0.08-0.2 | 84-89 | |
| 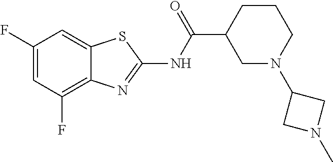 [SMILES: CN1CC(N2CCCC(C(=O)Nc3nc4c(F)cc(F)cc4s3)C2)C1] | JUF-S4-029 | 0.07 | 83 | 5.11 |
| 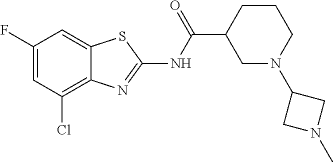 [SMILES: CN1CC(N2CCCC(C(=O)Nc3nc4c(Cl)cc(F)cc4s3)C2)C1] | | | | |
| 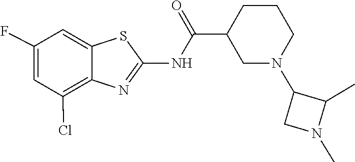 [SMILES: CC1C(N2CCCC(C(=O)Nc3nc4c(Cl)cc(F)cc4s3)C2)CN1C] | | | | |
| 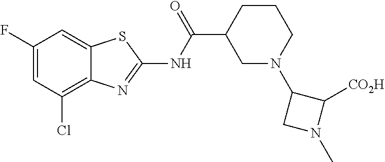 [SMILES: CN1CC(N2CCCC(C(=O)Nc3nc4c(Cl)cc(F)cc4s3)C2)C1C(=O)O] | | | | |
| 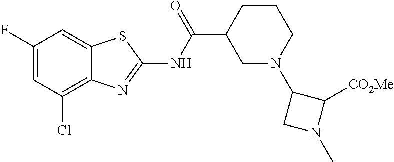 [SMILES: COC(=O)C1C(N2CCCC(C(=O)Nc3nc4c(Cl)cc(F)cc4s3)C2)CN1C] | | | | |
| 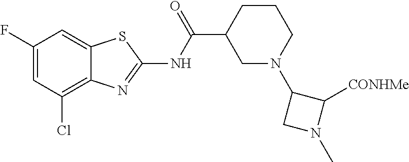 | | | | |
| 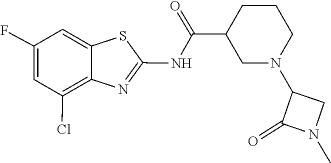 [SMILES: CN1CC(N2CCCC(C(=O)Nc3nc4c(Cl)cc(F)cc4s3)C2)C1=O] | | | | |
| 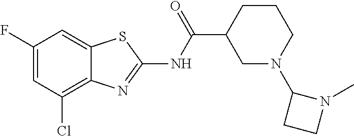 [SMILES: CN1CCC1N1CCCC(C(=O)Nc2nc3c(Cl)cc(F)cc3s2)C1] | | | | |
| 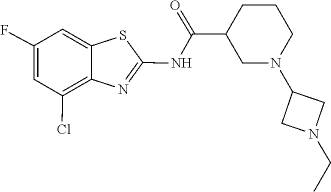 [SMILES: CCN1CC(N2CCCC(C(=O)Nc3nc4c(Cl)cc(F)cc4s3)C2)C1] | | | | |
| 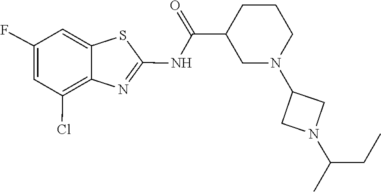 [SMILES: CCC(C)N1CC(N2CCCC(C(=O)Nc3nc4c(Cl)cc(F)cc4s3)C2)C1] | | | | |
| 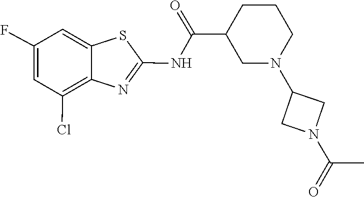 [SMILES: CC(=O)N1CC(N2CCCC(C(=O)Nc3nc4c(Cl)cc(F)cc4s3)C2)C1] | | | | |
| 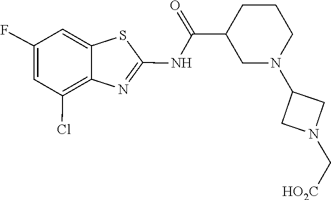 [SMILES: O=C(O)CN1CC(N2CCCC(C(=O)Nc3nc4c(Cl)cc(F)cc4s3)C2)C1] | | | | |
| 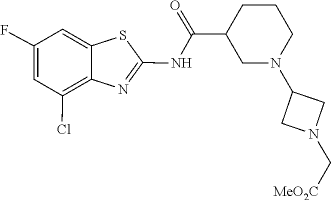 [SMILES: COC(=O)CN1CC(N2CCCC(C(=O)Nc3nc4c(Cl)cc(F)cc4s3)C2)C1] | | | | |
| 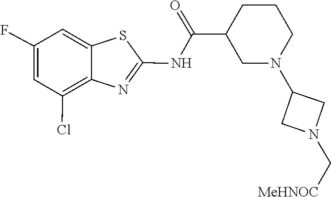 | | | | |
| 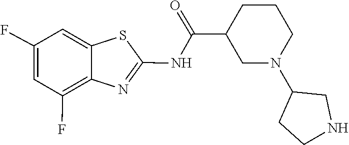 [SMILES: O=C(Nc1nc2c(F)cc(F)cc2s1)C1CCCN(C2CCNC2)C1] | JUF-S19- 005A | 1.3 | 72 | |
| 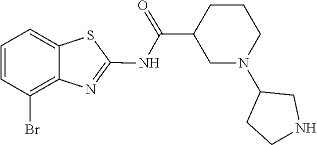 [SMILES: O=C(Nc1nc2c(Br)cccc2s1)C1CCCN(C2CCNC2)C1] | JUF-S21- 001A | 0.5 | 84 | |
| 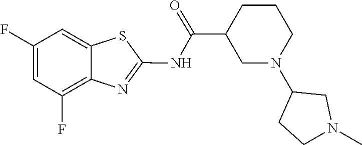 [SMILES: CN1CCC(N2CCCC(C(=O)Nc3nc4c(F)cc(F)cc4s3)C2)C1] | JUF-S4-015 | 0.13 | 89 | 4.09- 4.39 |
| 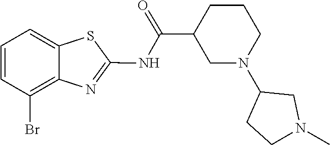 [SMILES: CN1CCC(N2CCCC(C(=O)Nc3nc4c(Br)cccc4s3)C2)C1] | JUF-S21- 001 | 0.25 | 84 | |
| 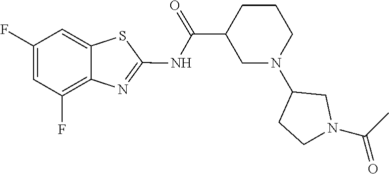 [SMILES: CC(=O)N1CCC(N2CCCC(C(=O)Nc3nc4c(F)cc(F)cc4s3)C2)C1] | | | | |
| 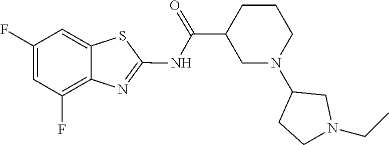 [SMILES: CCN1CCC(N2CCCC(C(=O)Nc3nc4c(F)cc(F)cc4s3)C2)C1] | | | | |
| 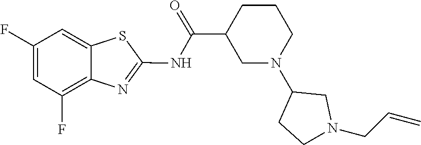 [SMILES: C=CCN1CCC(N2CCCC(C(=O)Nc3nc4c(F)cc(F)cc4s3)C2)C1] | JUF-S19- 006 | 0.05 | 89 | |
| 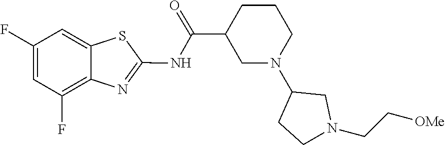 [SMILES: COCCN1CCC(N2CCCC(C(=O)Nc3nc4c(F)cc(F)cc4s3)C2)C1] | JUF-S19- 007 | 0.09 | 86 | |
| 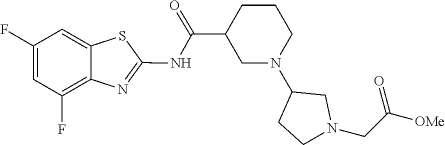 [SMILES: COC(=O)CN1CCC(N2CCCC(C(=O)Nc3nc4c(F)cc(F)cc4s3)C2)C1] | JUF-S19- 009 | 1 | 93 | |
| 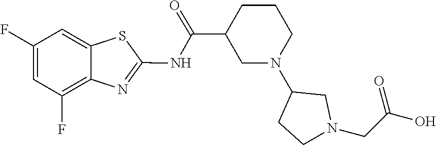 [SMILES: O=C(O)CN1CCC(N2CCCC(C(=O)Nc3nc4c(F)cc(F)cc4s3)C2)C1] | JUF-S19- 009 | >5 | | |
| 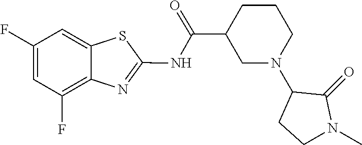 [SMILES: CN1CCC(N2CCCC(C(=O)Nc3nc4c(F)cc(F)cc4s3)C2)C1=O] | JUF-S23- 009 | 0.7 | 89 | |
| 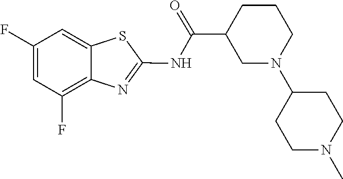 [SMILES: CN1CCC(N2CCCC(C(=O)Nc3nc4c(F)cc(F)cc4s3)C2)CC1] | JUF-S4-017 | 0.2 | 93 | |
| 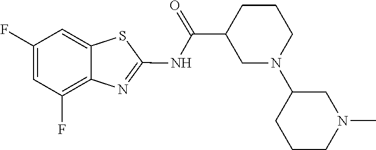 [SMILES: CN1CCCC(N2CCCC(C(=O)Nc3nc4c(F)cc(F)cc4s3)C2)C1] | JUF-S4-016 | 0.1 | 97 | |
| 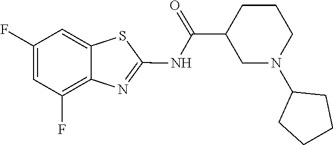 [SMILES: O=C(Nc1nc2c(F)cc(F)cc2s1)C1CCCN(C2CCCC2)C1] | JUF-S19- 013 | 0.6 | 91 | |
| 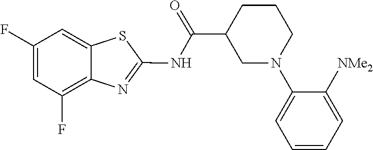 [SMILES: CN(C)c1ccccc1N1CCCC(C(=O)Nc2nc3c(F)cc(F)cc3s2)C1] | | | | |
| 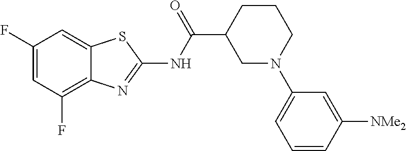 [SMILES: CN(C)c1cccc(N2CCCC(C(=O)Nc3nc4c(F)cc(F)cc4s3)C2)c1] | | | | |
| 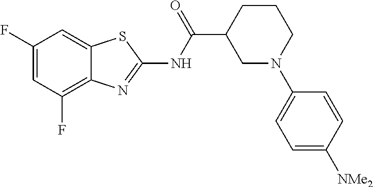 [SMILES: CN(C)c1ccc(N2CCCC(C(=O)Nc3nc4c(F)cc(F)cc4s3)C2)cc1] | JUF-S4-009 | 1.9 | 77 | |
| 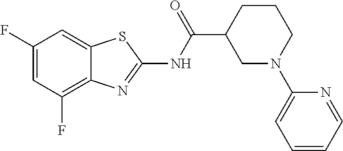 [SMILES: O=C(Nc1nc2c(F)cc(F)cc2s1)C1CCCN(c2ccccn2)C1] | JUF-S4-024 | >5 | | |
| 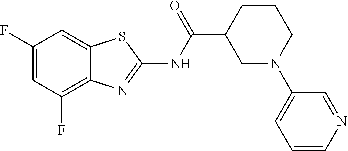 [SMILES: O=C(Nc1nc2c(F)cc(F)cc2s1)C1CCCN(c2cccnc2)C1] | JUF-S4-025 | 2.5-7.4 | 104-135 | |
| 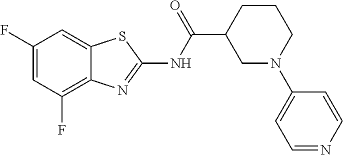 [SMILES: O=C(Nc1nc2c(F)cc(F)cc2s1)C1CCCN(c2ccncc2)C1] | JUF-S4-026 | 0.1 | 87 | 3.28- 3.72 |
| 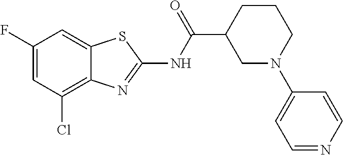 [SMILES: O=C(Nc1nc2c(Cl)cc(F)cc2s1)C1CCCN(c2ccncc2)C1] | | | | |
| 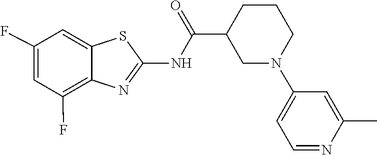 [SMILES: Cc1cc(N2CCCC(C(=O)Nc3nc4c(F)cc(F)cc4s3)C2)ccn1] | JUF-S22- 001 | 0.09 | 87 | |
| 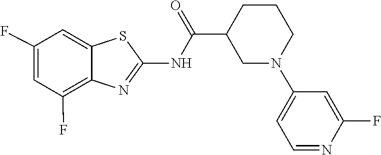 [SMILES: O=C(Nc1nc2c(F)cc(F)cc2s1)C1CCCN(c2ccnc(F)c2)C1] | JUF-S22- 002 | 3.5 | 101 | |
| 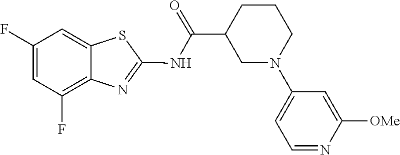 [SMILES: COc1cc(N2CCCC(C(=O)Nc3nc4c(F)cc(F)cc4s3)C2)ccn1] | JUF-S22- 003 | 2.1 | 101 | |
| 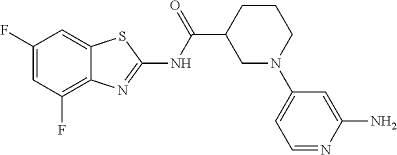 [SMILES: Nc1cc(N2CCCC(C(=O)Nc3nc4c(F)cc(F)cc4s3)C2)ccn1] | JUF-S22- 004 | 0.6 | 88 | |
| 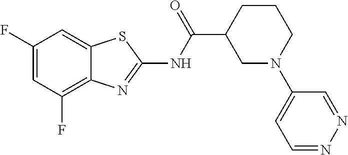 [SMILES: O=C(Nc1nc2c(F)cc(F)cc2s1)C1CCCN(c2ccnnc2)C1] | JUF-S22- 005 | 0.2-1.08 | 74-82 | |
| 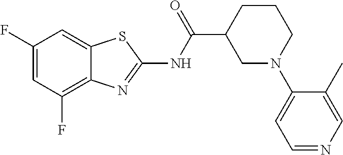 [SMILES: Cc1cnccc1N1CCCC(C(=O)Nc2nc3c(F)cc(F)cc3s2)C1] | | | | |
| 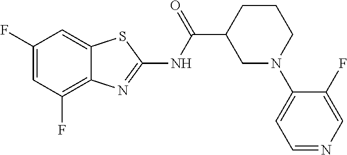 [SMILES: O=C(Nc1nc2c(F)cc(F)cc2s1)C1CCCN(c2ccncc2F)C1] | JUF-S22- 008 | 5.0 | 97 | |
| 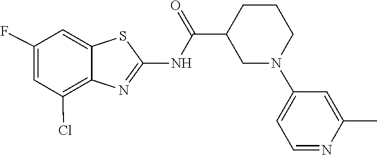 [SMILES: Cc1cc(N2CCCC(C(=O)Nc3nc4c(Cl)cc(F)cc4s3)C2)ccn1] | | | | |
| 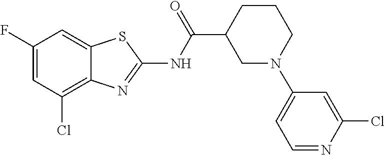 [SMILES: O=C(Nc1nc2c(Cl)cc(F)cc2s1)C1CCCN(c2ccnc(Cl)c2)C1] | | | | |
| 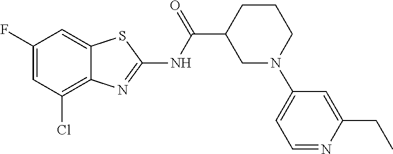 [SMILES: CCc1cc(N2CCCC(C(=O)Nc3nc4c(Cl)cc(F)cc4s3)C2)ccn1] | | | | |
| 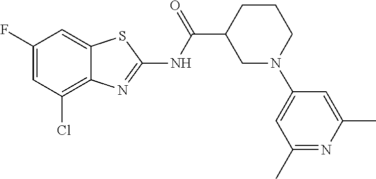 [SMILES: Cc1cc(N2CCCC(C(=O)Nc3nc4c(Cl)cc(F)cc4s3)C2)cc(C)n1] | | | | |
| 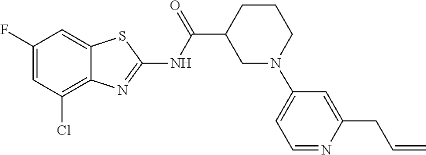 [SMILES: C=CCc1cc(N2CCCC(C(=O)Nc3nc4c(Cl)cc(F)cc4s3)C2)ccn1] | | | | |
| 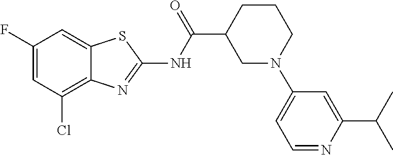 [SMILES: CC(C)c1cc(N2CCCC(C(=O)Nc3nc4c(Cl)cc(F)cc4s3)C2)ccn1] | | | | |
| 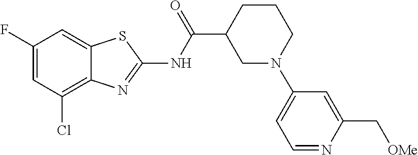 [SMILES: COCc1cc(N2CCCC(C(=O)Nc3nc4c(Cl)cc(F)cc4s3)C2)ccn1] | | | | |
| 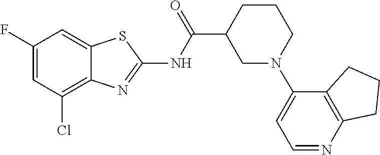 [SMILES: O=C(Nc1nc2c(Cl)cc(F)cc2s1)C1CCCN(c2ccnc3c2CCC3)C1] | | | | |
| 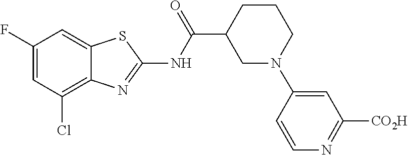 [SMILES: O=C(O)c1cc(N2CCCC(C(=O)Nc3nc4c(Cl)cc(F)cc4s3)C2)ccn1] | | | | |
| 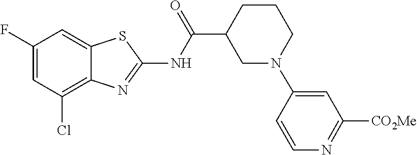 [SMILES: COC(=O)c1cc(N2CCCC(C(=O)Nc3nc4c(Cl)cc(F)cc4s3)C2)ccn1] | | | | |
| 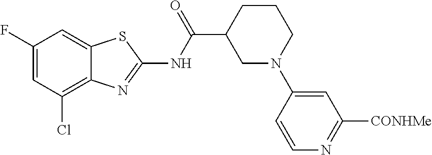 | | | | |
| 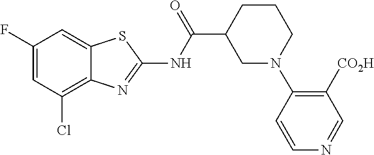 [SMILES: O=C(O)c1cnccc1N1CCCC(C(=O)Nc2nc3c(Cl)cc(F)cc3s2)C1] | | | | |
| 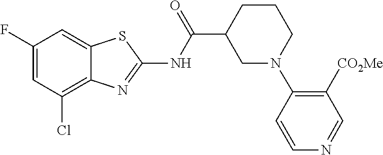 [SMILES: COC(=O)c1cnccc1N1CCCC(C(=O)Nc2nc3c(Cl)cc(F)cc3s2)C1] | | | | |
| 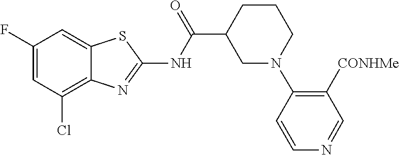 | | | | |
| 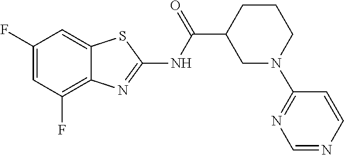 [SMILES: O=C(Nc1nc2c(F)cc(F)cc2s1)C1CCCN(c2ccncn2)C1] | JUF-S22- 005 | 0.2 | 82 | |
| 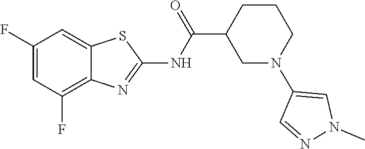 [SMILES: Cn1cc(N2CCCC(C(=O)Nc3nc4c(F)cc(F)cc4s3)C2)cn1] | JUF-S4-023 | 2.1-4.9 | 81-86 | |
| 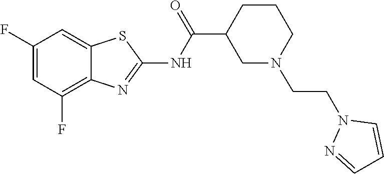 [SMILES: O=C(Nc1nc2c(F)cc(F)cc2s1)C1CCCN(CCn2cccn2)C1] | JUF-S4-012 | 0.7 | 88 | 2.91 |
| 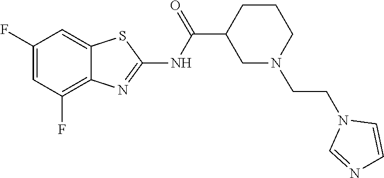 [SMILES: O=C(Nc1nc2c(F)cc(F)cc2s1)C1CCCN(CCn2ccnc2)C1] | JUF-S4-013 | 0.9 | 89 | |
| 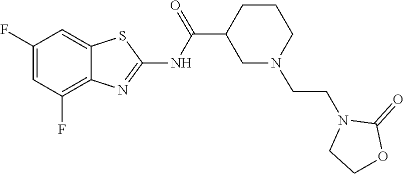 [SMILES: O=C(Nc1nc2c(F)cc(F)cc2s1)C1CCCN(CCN2CCOC2=O)C1] | JUF-S4- 005A | 1.6 | 93 | |
| 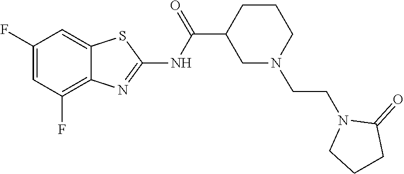 [SMILES: O=C(Nc1nc2c(F)cc(F)cc2s1)C1CCCN(CCN2CCCC2=O)C1] | JUF-S4-014 | 1.8 | 105 | |

TABLE 2
Modulation of pGC-A for compounds of Formula (I-b).

| | | EC₅₀ | | |
|---|---|---|---|---|
| Compound Structure | ID | (μM) | Eₘₐₓ (%) | LLE |
| 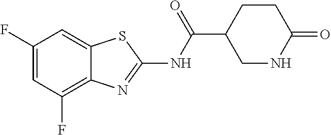 [SMILES: O=C1CCC(C(=O)Nc2nc3c(F)cc(F)cc3s2)CN1] | JUF-S11- 001A | 1.01 | 58 | |
| 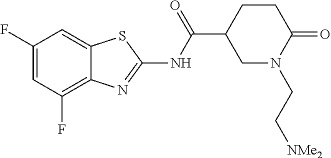 [SMILES: CN(C)CCN1CC(C(=O)Nc2nc3c(F)cc(F)cc3s2)CCC1=O] | JUF-S11- 001 | >5 | | |
| 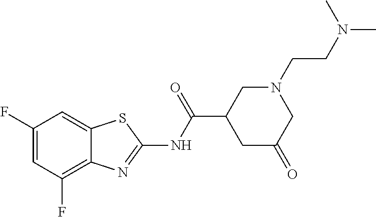 [SMILES: CN(C)CCN1CC(=O)CC(C(=O)Nc2nc3c(F)cc(F)cc3s2)C1] | | | | |

TABLE 3
Modulation of pGC-A for additional compounds of the invention.

| | | IC₅₀ | Eₘₐₓ | |
|---|---|---|---|---|
| Compound Structure | ID | (μM) | (%) | LLE |
| 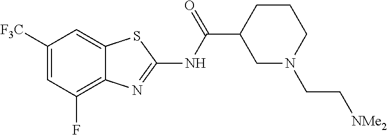 [SMILES: CN(C)CCN1CCCC(C(=O)Nc2nc3c(F)cc(C(F)(F)F)cc3s2)C1] | JUF-S3-004 | >5 | | |
| 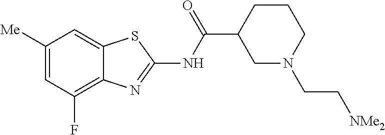 [SMILES: Cc1cc(F)c2nc(NC(=O)C3CCCN(CCN(C)C)C3)sc2c1] | JUF-S3-005 | 0.9 | 101 | |
| 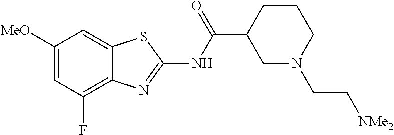 [SMILES: COc1cc(F)c2nc(NC(=O)C3CCCN(CCN(C)C)C3)sc2c1] | JUF-S3-006 | 6.7 | 112 | |
| 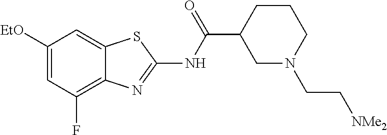 [SMILES: CCOc1cc(F)c2nc(NC(=O)C3CCCN(CCN(C)C)C3)sc2c1] | JUF-S3-010 | >5 | | |
| 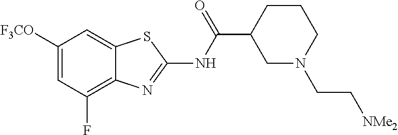 [SMILES: CN(C)CCN1CCCC(C(=O)Nc2nc3c(F)cc(OC(F)(F)F)cc3s2)C1] | | | | |
| 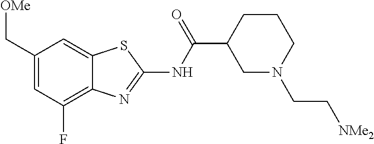 [SMILES: COCc1cc(F)c2nc(NC(=O)C3CCCN(CCN(C)C)C3)sc2c1] | | | | |
| 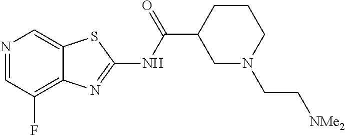 [SMILES: CN(C)CCN1CCCC(C(=O)Nc2nc3c(F)cncc3s2)C1] | | | | |
| 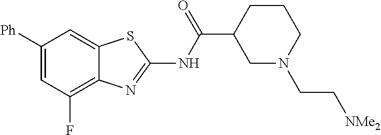 [SMILES: CN(C)CCN1CCCC(C(=O)Nc2nc3c(F)cc(-c4ccccc4)cc3s2)C1] | | | | |
| 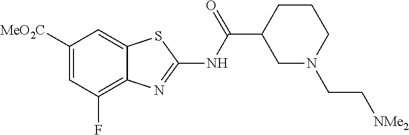 [SMILES: COC(=O)c1cc(F)c2nc(NC(=O)C3CCCN(CCN(C)C)C3)sc2c1] | | | | |
| 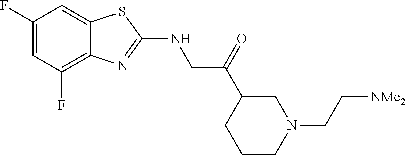 [SMILES: CN(C)CCN1CCCC(C(=O)CNc2nc3c(F)cc(F)cc3s2)C1] | | | | |
| 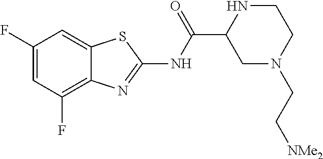 [SMILES: CN(C)CCN1CCNC(C(=O)Nc2nc3c(F)cc(F)cc3s2)C1] | | | | |
| 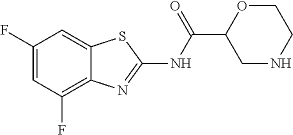 [SMILES: O=C(Nc1nc2c(F)cc(F)cc2s1)C1CNCCO1] | JUF-S11- 003A | >5 | | |
| 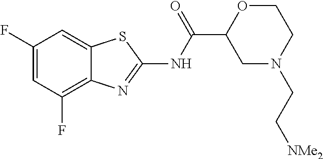 [SMILES: CN(C)CCN1CCOC(C(=O)Nc2nc3c(F)cc(F)cc3s2)C1] | JUF-S11-003 | >5 | | |
| 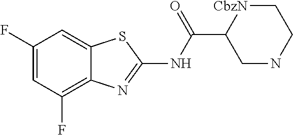 [SMILES: O=C(Nc1nc2c(F)cc(F)cc2s1)C1CNCCN1C(=O)OCc1ccccc1] | JUF-S11- 002A | >5 | | |

### Example 2: Synthesis of Compounds

Compounds delineated herein can be made using standard chemical synthesis procedures and reagents known to one of ordinary skill in the art and as described herein. Representative synthetic schemes are shown below.

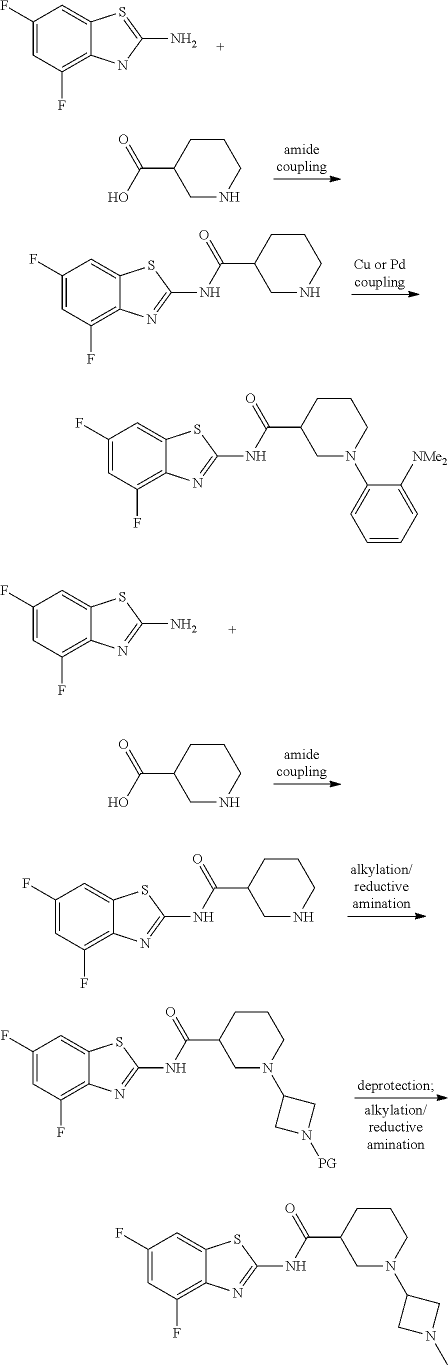

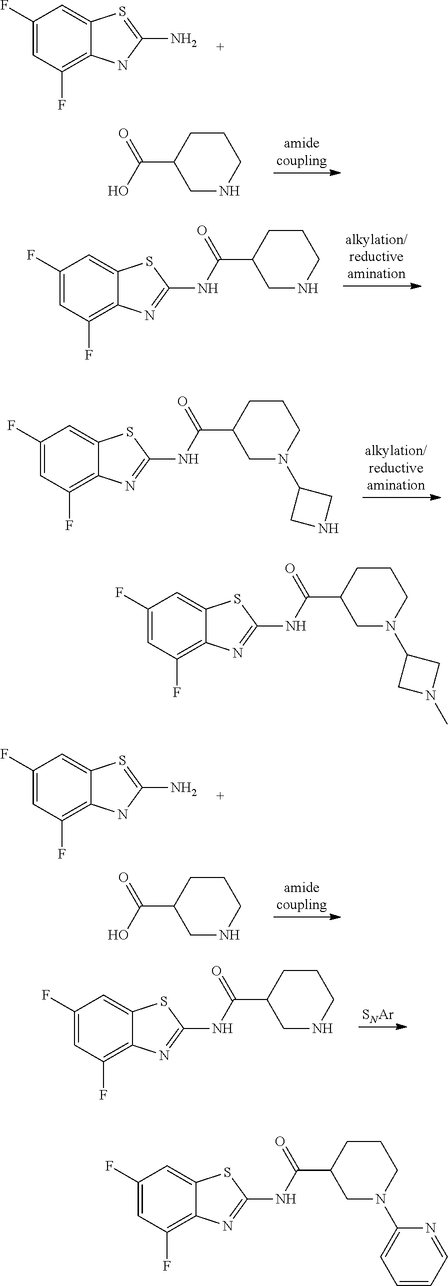

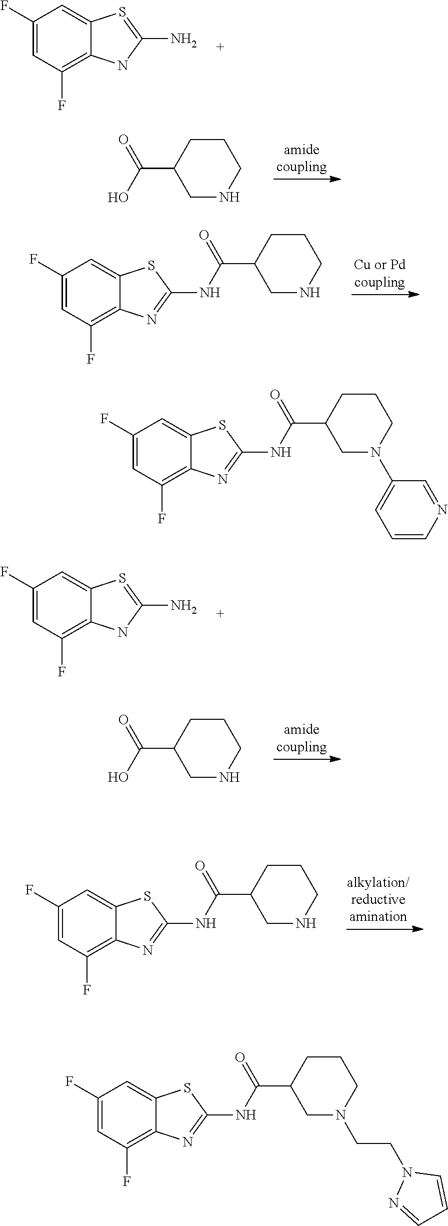

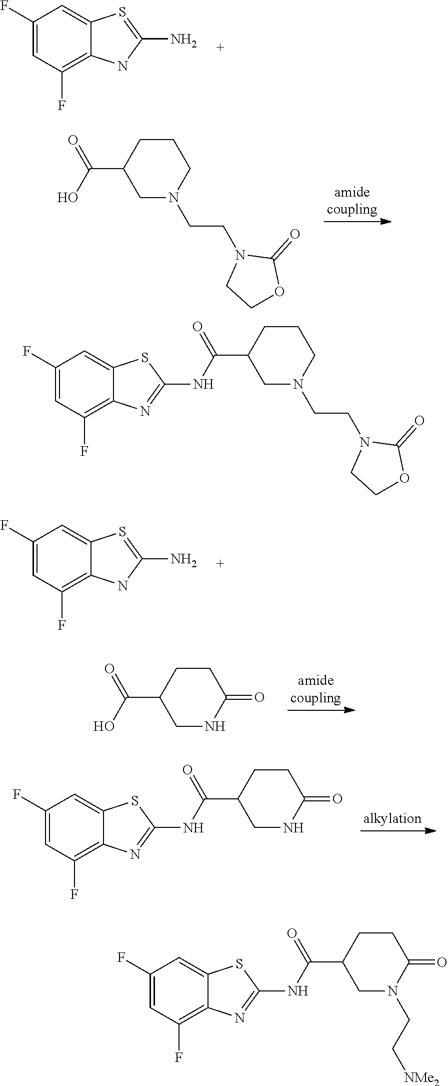

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

1. A compound of Formula (I-a): 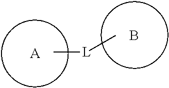
or a pharmaceutically acceptable salt, solvate, hydrate, polymorph, co-crystal, tautomer, stereoisomer, isotopically labeled compound, or prodrug thereof, wherein:
Ring B is of formula: 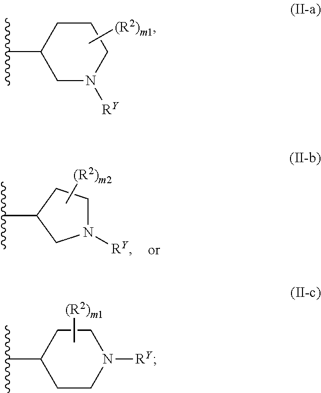
L is of formula: 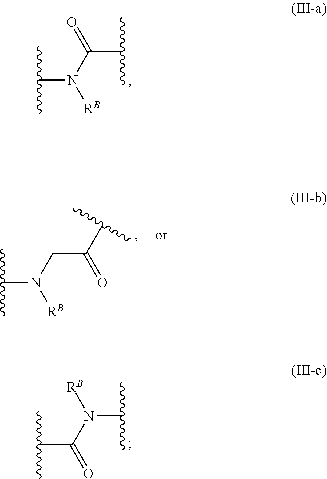
Ring A is of formula: 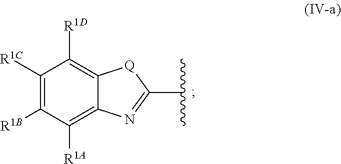
Q is oxygen, sulfur, or NR^{B};
R^{1A} is halogen;
each of R^{1B}, R^{1C}, and R^{1D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; or R^{1B} and R^{1C} or R^{1C} and R^{1D} are taken together to form an optionally substituted carbocyclyl, R^{1B} and R^{1C} or R^{1C} and R^{1D} are taken together to form an optionally substituted heterocyclyl, R^{1B} and R^{1C} or R^{1C} and R^{1D} are taken together to form an optionally substituted aryl, or R^{1B} and R^{1C} or R^{1C} and R^{1D} are taken together to form an optionally substituted heteroaryl;
each occurrence of R² is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; or two occurrences of R² are taken together to form ═O or ═S, two occurrences of R² are taken together to form an optionally substituted carbocyclyl, or two occurrences of R² are taken together to form an optionally substituted heterocyclyl;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring;
each occurrence of R^{B} is hydrogen, optionally substituted alkyl, optionally substituted acyl, or a nitrogen protecting group;
R^{Y} is optionally substituted carbocyclyl, optionally substituted heterocyclyl containing a single nitrogen atom in the heterocyclyl, optionally substituted aryl, optionally substituted heteroaryl, optionally substituted alkyl substituted with optionally substituted heteroaryl, or optionally substituted alkyl substituted with heterocyclyl comprising at least one ring oxo or thiooxo substituent in the heterocyclyl;
m1 is 0, 1, 2, 3, 4, 5, 6, 7, 8, or 9; and
m2 is 0, 1, 2, 3, 4, 5, 6, or 7.

2. The compound of claim 1, or a pharmaceutically acceptable salt, solvate, hydrate, polymorph, co-crystal, tautomer, stereoisomer, isotopically labeled compound, or prodrug thereof, wherein Ring B is of formula:

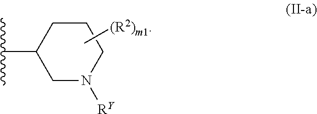

3. The compound of claim 1, or a pharmaceutically acceptable salt, solvate, hydrate, polymorph, co-crystal, tautomer, stereoisomer, isotopically labeled compound, or prodrug thereof, wherein Ring B is of formula:

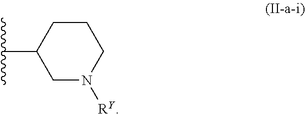

4-7. (canceled)

8. The compound of claim 1, or a pharmaceutically acceptable salt, solvate, hydrate, polymorph, co-crystal, tautomer, stereoisomer, isotopically labeled compound, or prodrug thereof, wherein R^{Y} is optionally substituted heterocyclyl containing a single nitrogen atom in the heterocyclyl.

9. The compound of claim 1, or a pharmaceutically acceptable salt, solvate, hydrate, polymorph, co-crystal, tautomer, stereoisomer, isotopically labeled compound, or prodrug thereof, wherein R^{Y} is optionally substituted azetidinyl, optionally substituted pyrrolidinyl, or optionally substituted piperidinyl.

10. The compound of claim 1, or a pharmaceutically acceptable salt, solvate, hydrate, polymorph, co-crystal, tautomer, stereoisomer, isotopically labeled compound, or prodrug thereof, wherein L is of formula:

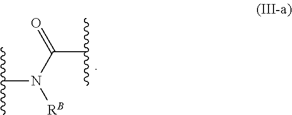

11. (canceled)

12. The compound of claim 1, or a pharmaceutically acceptable salt, solvate, hydrate, polymorph, co-crystal, tautomer, stereoisomer, isotopically labeled compound, or prodrug thereof, wherein Ring A is of formula:

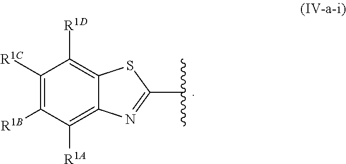

13. The compound of claim 1, or a pharmaceutically acceptable salt, solvate, hydrate, polymorph, co-crystal, tautomer, stereoisomer, isotopically labeled compound, or prodrug thereof, wherein Ring A is of formula: 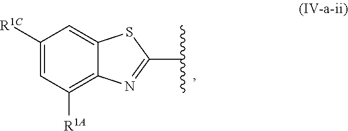
wherein R^{1C} is halogen.

14. The compound of claim 1, or a pharmaceutically acceptable salt, solvate, hydrate, polymorph, co-crystal, tautomer, stereoisomer, isotopically labeled compound, or prodrug thereof, wherein Ring A is of formula: 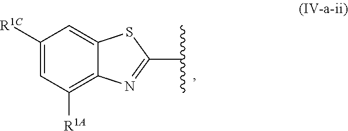
wherein R^{1C} is optionally substituted alkyl or —OR^{A}.

15. The compound of claim 1, wherein the compound of Formula (I-a) is: 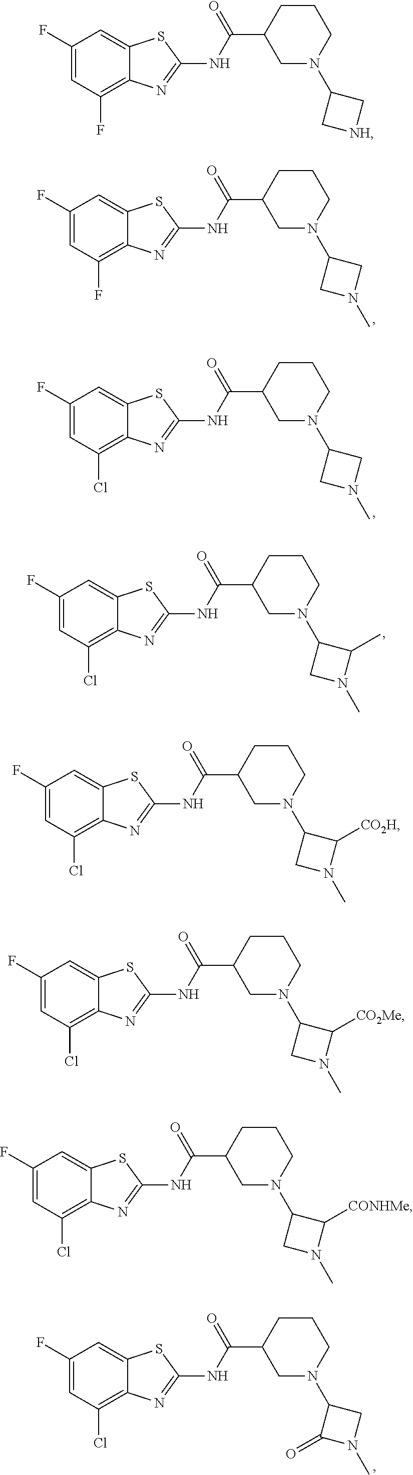 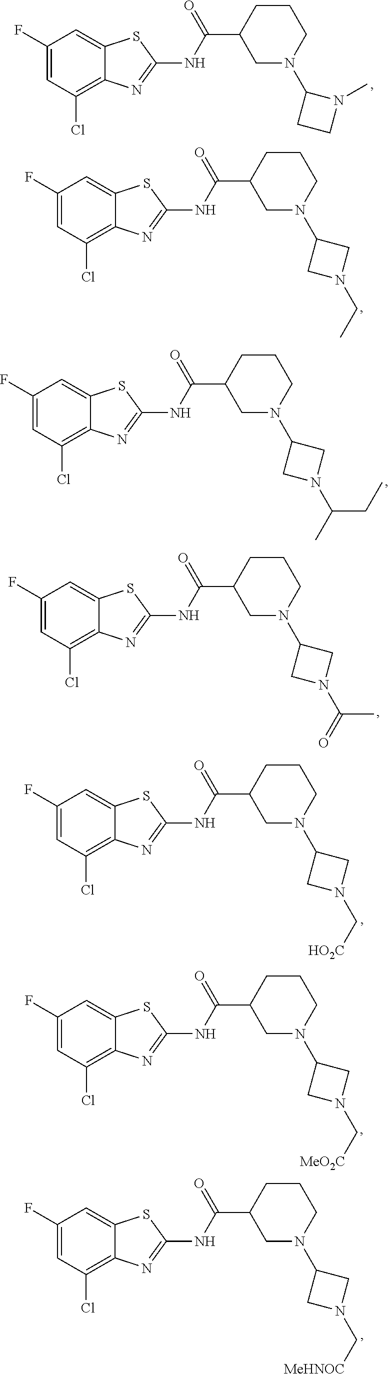 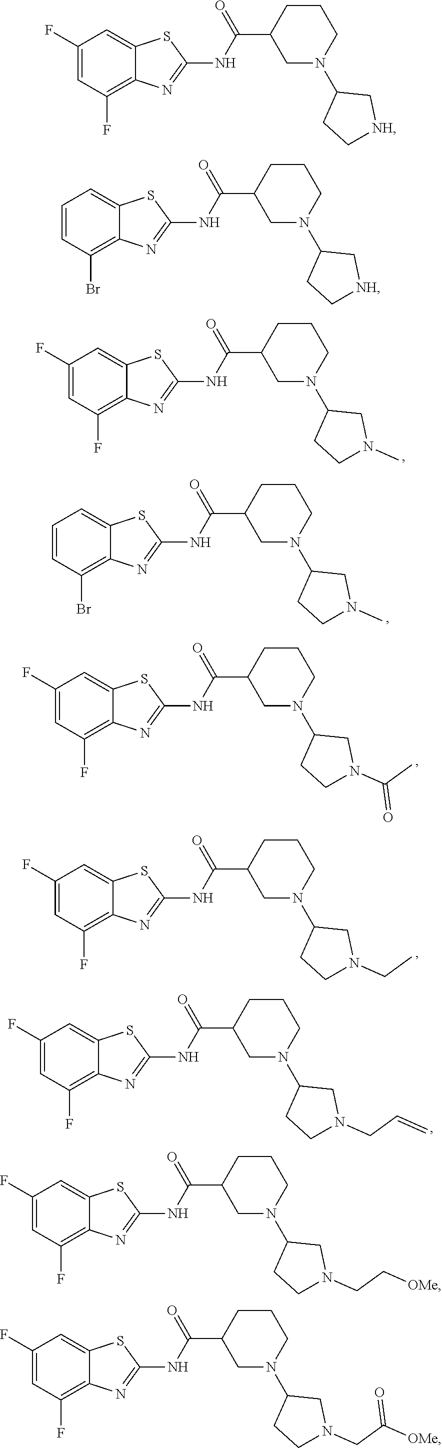 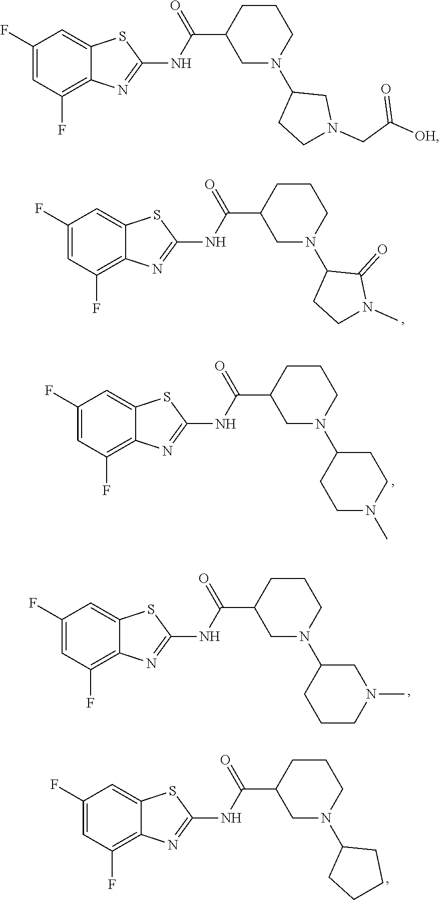
or a pharmaceutically acceptable salt, solvate, hydrate, polymorph, co-crystal, tautomer, stereoisomer, isotopically labeled compound, or prodrug thereof.

16. The compound of claim 1, or a pharmaceutically acceptable salt, solvate, hydrate, polymorph, co-crystal, tautomer, stereoisomer, isotopically labeled compound, or prodrug thereof, wherein R^{Y} is optionally substituted aryl or optionally substituted heteroaryl.

17-22. (canceled)

23. The compound of claim 1, wherein the compound of Formula (I-a) is: 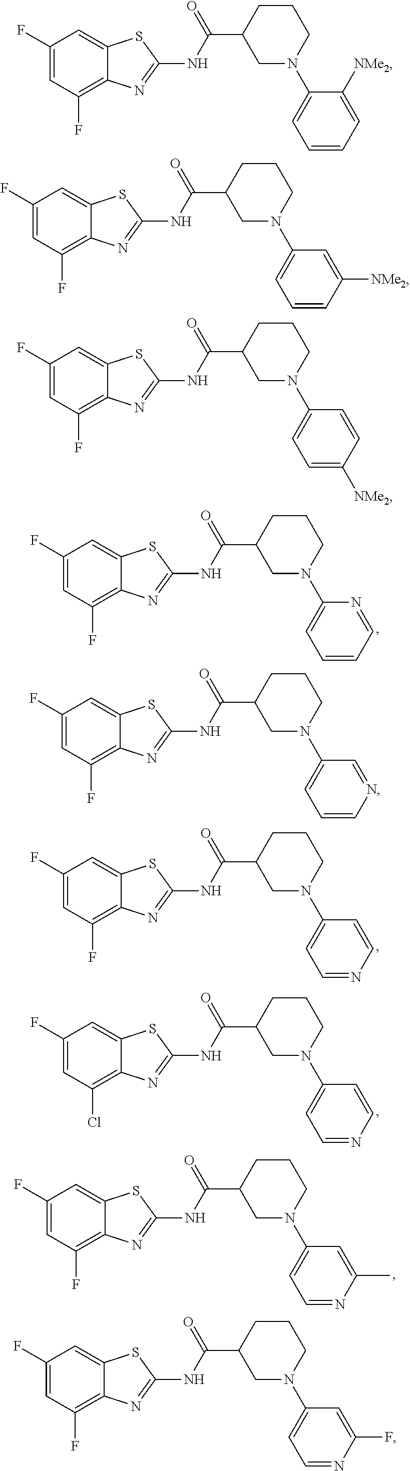 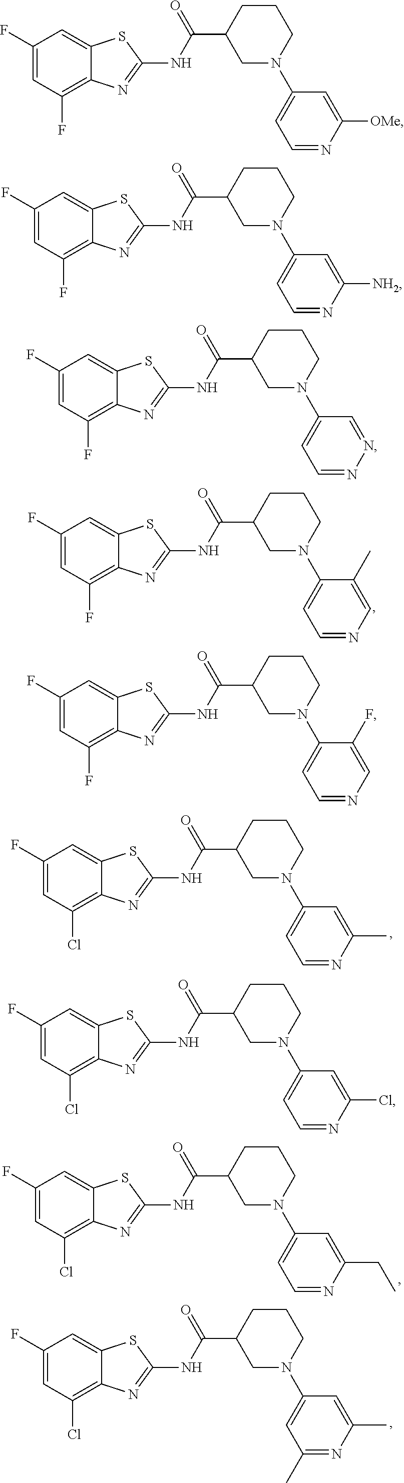 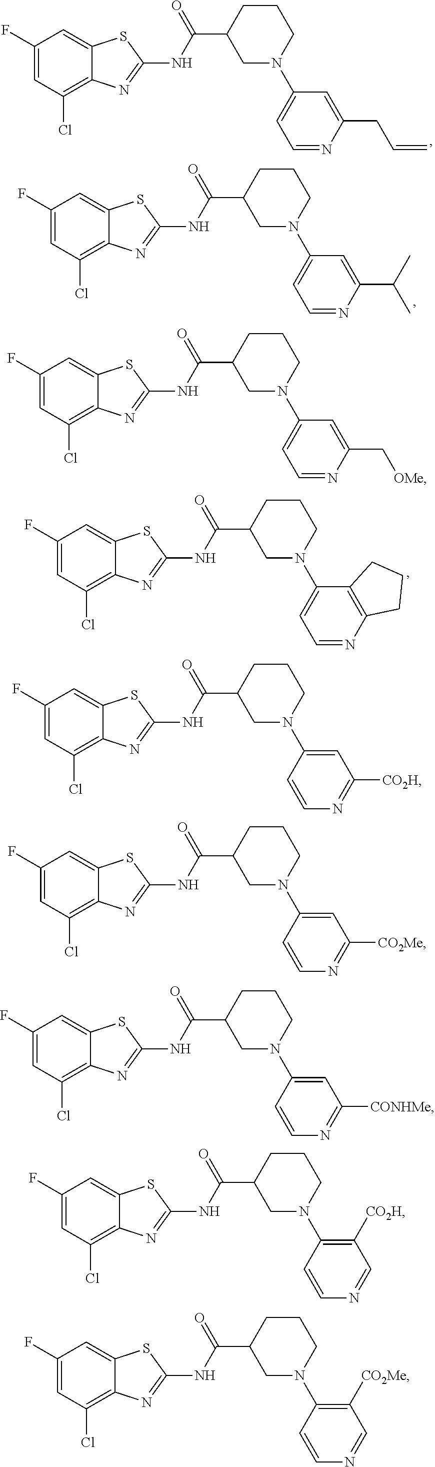 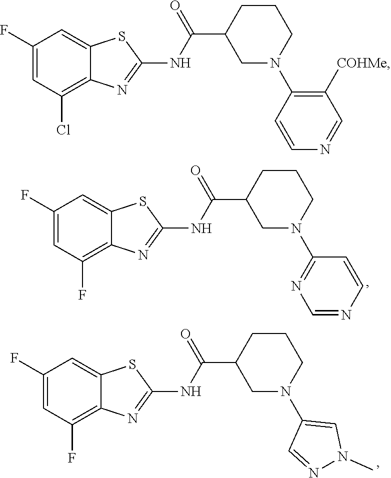
or a pharmaceutically acceptable salt, solvate, hydrate, polymorph, co-crystal, tautomer, stereoisomer, isotopically labeled compound, or prodrug thereof.

24. The compound of claim 1, or a pharmaceutically acceptable salt, solvate, hydrate, polymorph, co-crystal, tautomer, stereoisomer, isotopically labeled compound, or prodrug thereof, wherein R^{Y} is optionally substituted alkyl substituted with optionally substituted heteroaryl, or optionally substituted alkyl substituted with optionally substituted heterocyclyl comprising at least one ring oxo or thiooxo substituent in the heterocyclyl.

25-30. (canceled)

31. The compound of claim 1, wherein the compound of Formula (I-a) is: 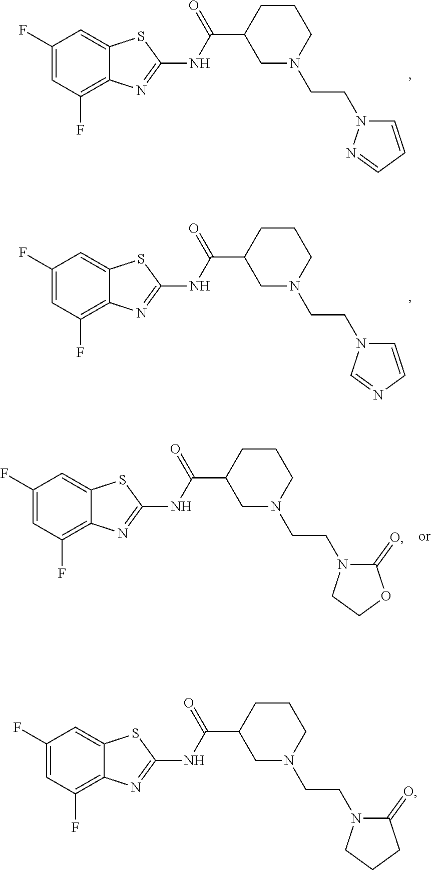 [SMILES: O=C(Nc1nc2c(F)cc(F)cc2s1)C1CCCN(CCn2cccn2)C1 ; O=C(Nc1nc2c(F)cc(F)cc2s1)C1CCCN(CCn2ccnc2)C1 ; O=C(Nc1nc2c(F)cc(F)cc2s1)C1CCCN(CCN2CCOC2=O)C1 ; "or" ; O=C(Nc1nc2c(F)cc(F)cc2s1)C1CCCN(CCN2CCCC2=O)C1]
or a pharmaceutically acceptable salt, solvate, hydrate, polymorph, co-crystal, tautomer, stereoisomer, isotopically labeled compound, or prodrug thereof.

32. A compound of Formula (I-b): 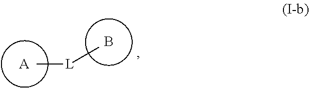
or a pharmaceutically acceptable salt, solvate, hydrate, polymorph, co-crystal, tautomer, stereoisomer, isotopically labeled compound, or prodrug thereof, wherein:
Ring B is optionally substituted 5-6 membered heterocyclyl-R^{X}, wherein the optionally substituted 5-6 membered heterocyclyl comprises at least one ring oxo or thiooxo substituent;
L is of formula: 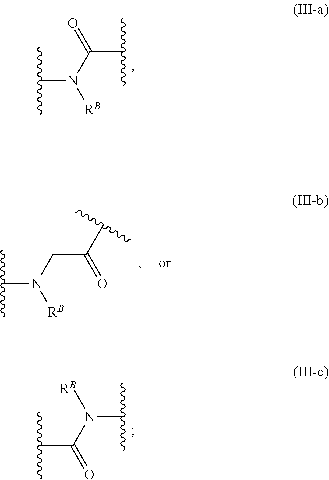
Ring A is of formula: 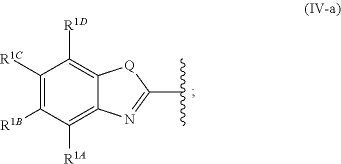
Q is oxygen, sulfur, or NR^{B};
R^{1A} is halogen;
each of R^{1B}, R^{1C}, and R^{1D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; or R^{1B} and R^{1C} or R^{1C} and R^{1D} are taken together to form an optionally substituted carbocyclyl, R^{1B} and R^{1C} or R^{1C} and R^{1D} are taken together to form an optionally substituted heterocyclyl, R^{1B} and R^{1C} or R^{1C} and R^{1D} are taken together to form an optionally substituted aryl, or R^{1B} and R^{1C} or R^{1C} and R^{1D} are taken together to form an optionally substituted heteroaryl;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring;
each occurrence of R^{B} is hydrogen, substituted or unsubstituted alkyl, substituted or unsubstituted acyl, or a nitrogen protecting group; and
R^{X} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

33-38. (canceled)

39. A pharmaceutical composition comprising the compound of claim 1, or a pharmaceutically acceptable salt, solvate, hydrate, polymorph, co-crystal, tautomer, stereoisomer, isotopically labeled compound, or prodrug thereof, and an excipient.

40. A method of treating or preventing a disease in a subject in need thereof, comprising administering to the subject in need thereof the compound of claim 1, or a pharmaceutically acceptable salt, solvate, hydrate, polymorph, co-crystal, tautomer, stereoisomer, isotopically labeled compound, or prodrug thereof.

41-49. (canceled)

50. A method of modulating particulate guanylyl cyclase-A (pGC-A) in a subject in need thereof or in a cell, tissue, or biological sample, comprising administering to the subject in need thereof or contacting the cell, tissue, or biological sample with an effective amount of the compound of claim 1, or a pharmaceutically acceptable salt, solvate, hydrate, polymorph, co-crystal, tautomer, stereoisomer, isotopically labeled compound, or prodrug thereof;
wherein the cell, tissue, or biological sample is in vivo.

51. A method of modulating particulate guanylyl cyclase-A (pGC-A) in a cell, tissue, or biological sample, comprising contacting the cell, tissue, or biological sample with an effective amount of the compound of claim 1, or a pharmaceutically acceptable salt, solvate, hydrate, polymorph, co-crystal, tautomer, stereoisomer, isotopically labeled compound, or prodrug thereof;
wherein the cell, tissue, or biological sample is in vitro.

52-55. (canceled)

56. A kit comprising the compound of claim 1, or a pharmaceutically acceptable salt, solvate, hydrate, polymorph, co-crystal, tautomer, stereoisomer, isotopically labeled compound, or prodrug thereof, and instructions for its use.
